(19) United States
(12) Patent Application Publication
(10) Pub. No.: US 2024/0424099 A1
(43) Pub. Date: Dec. 26, 2024

(54) TREATMENT OF CANCER WITH NK CELLS AND MULTISPECIFIC ENGAGERS

(71) Applicants: Artiva Biotherapeutics, Inc., San Diego, CA (US); Affimed GmbH, Mannheim (DE); GC Cell Corporation, Gyeonggi-do (KR)

(72) Inventors: Peter FLYNN, Cardiff by the Sea, CA (US); Jason B. LITTEN, Del Mar, CA (US); Thomas James FARRELL, La Jolla, CA (US); Heather Karen RAYMON, La Jolla, CA (US); Srinivas Sai SOMANCHI, San Diego, CA (US); Lisa GUERRETTAZ, Cardiff, CA (US); Thorsten GRAEF, Los Altos Hills, CA (US); Joachim KOCH, Heidelberg (DE); Jens PAHL, Heidelberg (DE); Bokyung MIN, Gyeonggi-do (KR); Hyojin KIM, Gyeonggi-do (KR); Sanghyun LEE, Gyeonggi-do (KR)

(21) Appl. No.: 18/707,276

(22) PCT Filed: Nov. 3, 2022
(86) PCT No.: PCT/US2022/048887

§ 371 Date: May 3, 2024

Related U.S. Application Data
(60) Provisional application No. 63/275,890, filed on Nov. 4, 2021.

(51) Int. Cl. A61K 39/00; A61K 38/20; A61K 39/395; A61K 47/20; A61K 47/26; A61K 47/36; A61K 47/42; A61P 35/00; C12N 5/0783

(52) U.S. Cl. CPC A61K 39/4613; A61K 38/2013; A61K 39/39558; A61K 39/4637; A61K 47/20; A61K 47/26; A61K 47/36; A61K 47/42; A61P 35/00; C12N 5/0646; A61K 2039/545; C12N 2501/2302; C12N 2501/2321; C12N 2501/25; C12N 2502/1114

(57) ABSTRACT

Provided herein are methods for treating a patient suffering from cancer with an natural killer (NK) cell with a KIR-B haplotype and expresses a CD16 molecule, and a bispecific antibody which binds CD16 and CD30, along with methods of producing NK cell populations, and pharmaceutical compositions comprising the NK cells and bispecific antibodies.

### CLAIM OF PRIORITY

This application claims the benefit of U.S. Provisional Application Ser. No. 63/275,890, filed on Nov. 4, 2021. The entire contents of the foregoing are incorporated herein by reference.

### BACKGROUND

Targeted therapies, including antibody therapy, have revolutionized cancer treatment. One mechanism of action by which antibody therapy induces cytotoxicity is through antibody dependent cell-mediated cytotoxicity (ADCC). Many cancer patients are unable to mount a robust ADCC response. A reduced ADCC response may render any of the indicated monoclonal antibody therapeutics significantly less effective for these patients, which could prevent these patients from responding or lead to relapse. Thus, a reduced ADCC response could negatively impact their clinical outcomes.

Despite recent discoveries and developments of several anti-cancer agents, there is still a need for improved methods and therapeutic agents due to poor prognosis for many types of cancers.

The present invention addresses these and other deficiencies in the art.

### SUMMARY

NK cells are immune cells that can engage tumor cells through a complex array of receptors on their cell surface, as well as through antibody-dependent cellular cytotoxicity (ADCC). To initiate ADCC, NK cells engage with antibodies via the CD16 receptor on their surface. NK cells may have an advantage over other immune cells, such as the T cells used in CAR-T cell therapy and other cell therapies. In an exemplary advantage, NK cells can be used as allogeneic therapies, meaning that NK cells from one donor can be safely used in one or many patients without the requirement for HLA matching, gene editing, or other genetic manipulations. Allogeneic NK cells with anti-tumor activity can be administered safely to patients without many of the risks associated with T cell therapies, such as severe cytokine release syndrome (CRS), and neurological toxicities or graft versus host disease (GvHD).

Allogeneic NK cells may provide an important treatment option for cancer patients. In one exemplary advantage, NK cells have been well tolerated without evidence of graft-versus-host disease, neurotoxicity or cytokine release syndrome associated with other cell-based therapies. In another exemplary advantage. NK cells do not require prior antigen exposure or expression of a specific antigen to identify and lyse tumor cells. In another exemplary advantage, NK cells have the inherent ability to bridge between innate immunity and engender a multi-clonal adaptive immune response resulting in long-term anticancer immune memory.

For example, NK cells can recruit and activate other components of the immune system. Activated NK cells secrete cytokines and chemokines, such as interferon gamma (IFNγ): tumor necrosis factor alpha (TNFα); and macrophage inflammatory protein 1 (MIP1) that signal and recruit T cells to tumors. Through direct killing of tumor cells, NK cells also expose tumor antigens for recognition by the adaptive immune system.

Additionally, cords with preferred characteristics for enhanced clinical activity (e.g., high-affinity CD16 and Killer cell Immunoglobulin-like Receptor (KIR) B-haplotype) can be selected by utilizing a diverse umbilical cord blood bank as a source for NK cells.

The administration of the allogenic NK cells, as described herein, can enhance patients' ADCC responses, e.g., in combination with a multispecific engager, e.g., a multispecific engager described herein.

CD30 is a cell membrane protein of the tumor necrosis factor receptor family universally expressed in classical Hodgkin lymphoma (HL) as well as in several sub-types of peripheral T-cell lymphomas, to varying degrees, including anaplastic large-cell lymphoma (ALCL), peripheral T-cell lymphoma (PTCL)—not otherwise specified (PTCL-NOS), and angioimmunoblastic T-cell lymphoma (AITL).

In classical HL (cHL), the most common CD30-positive lymphoma, frontline chemotherapy (ABVD or BEACOPP) with or without radiotherapy has demonstrated significant effectiveness. In patients with advanced cHL, frontline therapy may also include the CD30 targeting antibody drug conjugate brentuximab vedotin with chemotherapy (AVD). However, up to 30% patients are refractory to frontline treatment or relapse. For patients with relapsed or refractory cHL, 50% or fewer can be cured with high-dose chemotherapy and autologous stem cell transplantation (ASCT) (Majhail N S, Weisdorf D J, Defor T E, et al. Long-term results of autologous stem cell transplantation for primary refractory or relapsed Hodgkin's lymphoma. Biol Blood Marrow Transplant. 2006; 12 (10): 1065-1072. doi: 10.1016/j.bbmt.2006.06.006; Josting A. Müller H, Borchmann P, et al. Dose intensity of chemotherapy in patients with relapsed Hodgkin's lymphoma. J Clin Oncol. 2010; 28 (34): 5074-5080. doi: 10.1200/JCO.2010.30.5771; Bartlett N L, Herrera A F, Domingo-Domenech E, et al. A phase 1b study of AFM13 in combination with pembrolizumab in patients with relapsed or refractory Hodgkin lymphoma. Blood. 2020; 136 (21): 2401-2409. doi: 10.1182/blood.2019004701). Relapse after ASCT is associated with a poor prognosis with median survival of 26 months (Voorhees T J, Beaven A W, Therapeutic Updates for Relapsed and Refractory Classical Hodgkin Lymphoma. Cancers (Basel). 2020; 12 (10): 2887. Published 2020 Oct. 8. doi: 10.3390/cancers12102887). Systemic treatment options for refractory and relapsed patients may also include agents such as brentuximab vedotin either as monotherapy or in combination with another agent, and/or a PD-(L) 1 inhibitor. Despite recent advancements that have included promising targeted and immunological agents, there is still an unmet medical need for treatments in the relapsed/refractory setting that provide a high level of response, longer duration of response, and chance of cure, together with a clinically acceptable safety profile.

With respect to peripheral T-cell lymphomas, first-line treatment with CHOP or CHOP-like regimens results in poor outcomes, except in the case of ALK+ALCL. Despite intensified approaches in frontline therapy, such as consolidation with ASCT, these patients are still at considerable risk of relapse or early progression. In general, most if not all patients undergoing treatment for PTCL will not achieve remission or will relapse with very poor long-term survival, especially in absence of hematopoietic cell transplantation (HCT), with median PFS and OS estimates as low as 3 and 6 months, respectively (Mak V, Hamm J, Chhanabhai, et al. Survival of patients with peripheral T-cell lymphoma after first relapse or progression: spectrum of disease and rare long-term survivors. J. Clin. Oncol. 2013; 31 (16): 1970-1976; Biasoli I, Cesaretti M, Bellei M, et al. Dismal outcome of T-cell lymphoma patients failing first-line treatment: results of a population-based study from the Modena Cancer Registry. Hematol. Oncol. 2015; 33 (3): 147-151; Bellei M, Foss F, Shustov A, et al. The outcome of peripheral T-cell lymphoma patients failing first-line therapy: a report from the prospective, International T-Cell Project. Hematologica. 2018; 103 (7): 1191-1197). Therefore, novel therapeutics and treatment strategies are needed to address the unmet medical needs that exist for patients with PTCL.

AFM13 is a tetravalent bispecific (anti-human CD30×anti-human CD16A) recombinant antibody construct which is being investigated for the treatment of HL and other CD30-positive malignancies including PTCL. AFM13 targets CD30 antigen expressed on malignant lymphoma cells. At the same time, the anti-CD16A domains bind to CD16A (FcλRIIIA) on NK cells and macrophages. AFM13 forms a bridge between the tumor target cells and innate effector cells, triggering lysis of CD30 antigen-positive cells by NK cells via antibody-dependent cell-mediated cytotoxicity (ADCC).

It has been shown that NK cell populations are absent in the immunosuppressive tumor microenvironment of HL. Furthermore, NK cells from patients with HL are dysfunctional, due in part to an imbalance in activating and inhibitory receptors (Reiners K S, Kessler J. Sauer M. et al. Rescue of impaired NK cell activity in Hodgkin lymphoma with bispecific antibodies in vitro and in patients. Mol Ther 2013:21:895-903). Because of these limitations in autologous NK cell function, optimal NK immunotherapy for HL likely requires an allogeneic source.

Thus, provided herein are, among other things, methods for treating a patient suffering from a CD30+ cancer comprising: administering to the patient a first pharmaceutical composition comprising a natural killer cell (NK cell) comprising a KIR-B haplotype and expression of a CD16 molecule; and administering to the patient a second pharmaceutical composition comprising a bispecific antibody or antigen binding fragment thereof comprising a first binding domain that specifically binds to CD16 (FcγRIII) and a second binding domain that specifically binds to CD30, wherein the first binding domain that specifically binds to CD16 comprises: a light chain variable domain (VL_CD16A) comprising a light chain complementarity determining region 1 (CDRL1) comprising SEQ ID NO: 9, a light chain complementarity determining region 2 (CDRL2) comprising SEQ ID NO: 10: a light chain complementarity determining region 3 (CDRL3) comprising SEQ ID NO: 11; and a heavy chain variable domain (VH_CD16A) comprising a heavy chain complementarity determining region 1 (CDRH1 comprising SEQ ID NO: 6: a heavy chain complementarity determining region 2 (CDRH2) comprising SEQ ID NO: 7; and a heavy chain complementarity determining region 3 (CDRH3) comprising SEQ ID NO: 8; and wherein the second binding domain that specifically binds to CD30 comprises: a light chain variable domain (VL_CD30) comprising a light chain complementarity determining region 1 (CDRL1) comprising SEQ ID NO: 15, a light chain complementarity determining region 2 (CDRL2) comprising SEQ ID NO: 16: a light chain complementarity determining region 3 (CDRL3) comprising SEQ ID NO: 17; and a heavy chain variable domain (VH_CD30) comprising a heavy chain complementarity determining region 1 (CDRH1 comprising SEQ ID NO: 12: a heavy chain complementarity determining region 2 (CDRH2) comprising SEQ ID NO: 13; and a heavy chain complementarity determining region 3 (CDRH3) comprising SEQ ID NO: 14.

In some embodiments, the NK cell is a cord blood-derived NK cell.

In some embodiments, the cord blood-derived NK cell has been produced by a method comprising: (a) providing a sample of cord blood cells comprising natural killer cells; (b) depleting the cells of CD3 (+) cells or enriching the seed cells for NK cells by positive selection; (c) expanding the natural killer cells by culturing the seed cells with a first plurality of cells from an inactivated CD4 (+) T cell line in a medium comprising IL-2, to produce the cord blood-derived natural killer cell.

In some embodiments, the inactivated CD4 (+) T cell line expresses at least one gene selected from the group consisting of a 4-1BBL gene, a membrane-bound IL-21 (mbIL-21) gene, an OX40L gene, and a mutated TNF-α gene. In some embodiments, the inactivated CD4 (+) T cell line expresses a 4-1BBL gene, a membrane-bound IL-21 (mbIL-21) gene, and a mutated TNF-α gene.

In some embodiments, the medium comprising IL-2 further comprises a T-cell stimulating antibody selected from the group consisting of OKT3, UCHT1, HTa, or a combination thereof.

In some embodiments, the CD16 molecule is a CD16A molecule. In some embodiments, the CD16 molecule comprises a V/V polymorphism at F158. In some embodiments, the first binding domain that specifically binds to CD16 specifically binds to CD16A.

In some embodiments, the natural killer cell is a population of natural killer cells. In some embodiments, the population of natural killer cells comprises at least 60%, e.g., at least 70%, at least 80%, at least 90% at least 95%, at least 99%, or 100% CD16+ cells. In some embodiments, the population of natural killer cells comprises at least 60%, e.g., at least 70%, at least 80%, at least 90% at least 95%, at least 99%, or 100% NKG2D+ cells. In some embodiments, the population of natural killer cells comprises at least 60%, e.g., at least 70%, at least 80%, at least 90% at least 95%, at least 99%, or 100% NKp46+ cells. In some embodiments, the population of natural killer cells comprises at least 60%, e.g., at least 70%, at least 80%, at least 90% at least 95%, at least 99%, or 100% NKp30+ cells. In some embodiments, the population of natural killer cells comprises at least 60%, e.g., at least 70%, at least 80%, at least 90% at least 95%, at least 99%, or 100% DNAM-1+ cells. In some embodiments, the population of natural killer cells comprises at least 60%, e.g., at least 70%, at least 80%, at least 90% at least 95%, at least 99%, or 100% NKp44+ cells. In some embodiments, the population of natural killer cells comprises less than 20%, e.g., 10% or less, 5% or less, 1% or less, 0.5% or less, or 0% CD3+ cells. In some embodiments, the population of natural killer cells comprises less than 20% or less, e.g., 10% or less, 5% or less, 1% or less, 0.5% or less, or 0% CD14+ cells. In some embodiments, the population of natural killer cells comprises less than 20% or less, e.g., 10% or less, 5% or less, 1% or less, 0.5% or less, or 0% CD19+ cells. In some embodiments, the population of natural killer cells comprises less than 20% or less, e.g., 10% or less, 5% or less, 1% or less, 0.5% or less, or 0% CD38+ cells.

In some embodiments, the population of NK cells comprises at least 100 million expanded natural killer cells, e.g., 200 million, 250 million, 300 million, 400 million, 500 million, 600 million, 700 million, 750 million, 800 million, 900 million, 1 billion, 2 billion, 3 billion, 4 billion, 5 billion, 6 billion, 7 billion, 8 billion, 9 billion, 10 billion, 15 billion, 20 billion, 25 billion, 50 billion, 75 billion, 80 billion, 9-billion, 100 billion, 200 billion, 250 billion, 300 billion, 400 billion, 500 billion, 600 billion, 700 billion, 800 billion, 900 billion, 1 trillion, 2 trillion, 3 trillion, 4 trillion, 5 trillion, 6 trillion, 7 trillion, 8 trillion, 9 trillion, or 10 trillion expanded natural killer cells.

In some embodiments, the population of NK cells is produced by a method comprising: (a) obtaining seed cells comprising natural killer cells from umbilical cord blood; (b) depleting the seed cells of CD3+ cells; (c) expanding the natural killer cells by culturing the depleted seed cells with a first plurality of Hut78 cells engineered to express a membrane bound IL-21, a mutated TNFα, and a 4-1BBL gene to produce expanded natural killer cells, thereby producing the population of natural killer cells. In some embodiments, the population of NK cells is produced by a method comprising: (a) obtaining seed cells comprising natural killer cells from umbilical cord blood; (b) depleting the seed cells of CD3+ cells; (c) expanding the natural killer cells by culturing the depleted seed cells with a first plurality of Hut78 cells engineered to express a membrane bound IL-21, a mutated TNFα, and a 4-1BBL gene to produce a master cell bank population of expanded natural killer cells; and (d) expanding the master cell bank population of expanded natural killer cells by culturing with a second plurality of Hut78 cells engineered to express a membrane bound IL-21, a mutated TNFα, and a 4-1BBL gene to produce expanded natural killer cells: thereby producing the population of natural killer cells.

In some embodiments, the population of NK cells is produced by a method further comprising, after step (c), (i) freezing the master cell bank population of expanded natural killer cells in a plurality of containers; and (ii) thawing a container comprising an aliquot of the master cell bank population of expanded natural killer cells, wherein expanding the master cell bank population of expanded natural killer cells in step (d) comprises expanding the aliquot of the master cell bank population of expanded natural killer cells.

In some embodiments, the umbilical cord blood is from a donor with the KIR-B haplotype and homozygous for the CD16 158V polymorphism.

In some embodiments, the population of NK cells is produced by a method comprising expanding the natural killer cells from umbilical cord blood at least 10,000 fold, e.g., 15,000 fold, 20,000 fold, 25,000 fold, 30,000 fold, 35,000 fold, 40,000 fold, 45,000 fold, 50,000 fold, 55,000 fold, 60,000 fold, 65,000 fold, or 70,000 fold.

In some embodiments, the population of natural killer cells is not enriched or sorted after expansion.

In some embodiments, the percentage of NK cells expressing CD16 in the population of natural killer cells is the same or higher than the percentage of natural killer cells in the seed cells from umbilical cord blood. In some embodiments, the percentage of NK cells expressing NKG2D in the population of natural killer cells is the same or higher than the percentage of natural killer cells in the seed cells from umbilical cord blood. In some embodiments, the percentage of NK cells expressing NKp30 in the population of natural killer cells is the same or higher than the percentage of natural killer cells in the seed cells from umbilical cord blood. In some embodiments, the percentage of NK cells expressing NKp44 in the population of natural killer cells is the same or higher than the percentage of natural killer cells in the seed cells from umbilical cord blood. In some embodiments, the percentage of NK cells expressing NKp46 in the population of natural killer cells is the same or higher than the percentage of natural killer cells in the seed cells from umbilical cord blood. In some embodiments, the percentage of NK cells expressing DNAM-1 in the population of natural killer cells is the same or higher than the percentage of natural killer cells in the seed cells from umbilical cord blood.

In some embodiments, the natural killer cell does not comprise a CD16 transgene. In some embodiments, the natural killer cell does not express an exogenous CD16 protein.

In some embodiments, the natural killer cell is not genetically engineered.

In some embodiments, the natural killer cell is derived from the same umbilical cord blood donor.

In some embodiments, the first pharmaceutical composition further comprises: (a) human albumin; (b) dextran; (c) glucose; (d) DMSO; and (e) a buffer. In some embodiments, the first pharmaceutical composition comprises from 30 to 50 mg/mL human albumin. In some embodiments, the first pharmaceutical composition comprises 50 mg/mL human albumin. In some embodiments, the first pharmaceutical composition comprises 20 to 30 mg/mL dextran. In some embodiments, the first pharmaceutical composition comprises 25 mg/mL dextran. In some embodiments, the dextran is Dextran 40. In some embodiments, the first pharmaceutical composition comprises from 12 to 15 mg/mL glucose. In some embodiments, the first pharmaceutical composition comprises 12.5 mg/mL glucose. In some embodiments, the first pharmaceutical composition comprises less than 27.5 g/L glucose. In some embodiments, the first pharmaceutical composition comprises from 50 to 60 ml/mL DMSO. In some embodiments, the first pharmaceutical composition comprises 55 mg/mL DMSO. In some embodiments, the first pharmaceutical composition comprises 40 to 60% v/v buffer. In some embodiments, the buffer is phosphate buffered saline. In some embodiments, the first pharmaceutical composition further comprises: (a) about 40 mg/mL human albumin; (b) about 25 mg/mL Dextran 40; (c) about 12.5 mg/mL glucose; (d) about 55 mg/mL DMSO; and (e) about 0.5 mL/mL phosphate buffered saline. In some embodiments, the first pharmaceutical composition further comprises 0.5 mL/mL water. In some embodiments, the first pharmaceutical composition further comprises a pharmaceutically acceptable excipient.

In some embodiments, the first binding domain that specifically binds to CD16 comprises a light chain variable (V_{L}) region comprising SEQ ID NO: 20 and a heavy chain variable (V_{H}) region comprising SEQ ID NO: 19. In some embodiments, the first binding domain that specifically binds to CD16 comprises a V_{L} region comprising an amino acid sequence having or having at least 85%, 86%, 87%, 88%, 89%, 90%, 91%, 92%, 93%, 94%, 95%, 96%, 97%, 98%, or 99% identity to SEQ ID NO: 20 and a Vu region comprising an amino acid sequence having or having at least 85%, 86%, 87%, 88%, 89%, 90%, 91%, 92%, 93%, 94%, 95%, 96%, 97%, 98%, or 99% identity to SEQ ID NO: 19. In some embodiments, the second binding domain that specifically binds to CD30 comprises a light chain variable (V_{L}) region comprising SEQ ID NO: 22 and a heavy chain variable (V_{H}) region comprising SEQ ID NO: 21. In some embodiments, the second binding domain that specifically binds to CD30 comprises a V_{L} region comprising an amino acid sequence having or having at least 85%, 86%, 87%, 88%, 89%, 90%, 91%, 92%, 93%, 94%, 95%, 96%, 97%, 98%, or 99% identity to SEQ ID NO: 22 and a V_{H} region comprising an amino acid sequence having or having at least 85%, 86%, 87%, 88%, 89%, 90%, 91%, 92%, 93%, 94%, 95%, 96%, 97%, 98%, or 99% identity to SEQ ID NO: 21.

In some embodiments, the bispecific antibody or antigen binding fragment thereof is a bispecific antigen binding fragment; and the variable domains of the bispecific antigen binding fragment are linked by peptide linkers L1, L2, and L3 from the N-terminus to the C-terminus in the order: VH_CD30-L1-VL_CD16A-L2-VH_CD16A-L3-VL_CD30. In some embodiments, the bispecific antibody or antigen binding fragment thereof is a bispecific antigen binding fragment; and the variable domains of the bispecific antigen binding fragment are linked by peptide linkers L1, L2, and L3 from the N-terminus to the C-terminus in the order: VH_CD16A-L1-VL_CD30-L2-VH_CD30-L3-VL_CD16A.

In some embodiments, each of peptide linkers L1, L2, and L3 consist of no more than 12 amino acid residues. In some embodiments, linker L2 of the antibody construct consists of between 3 and 9 amino acid residues, inclusive.

In some embodiments, the bispecific antibody or antigen binding fragment thereof is a comprises an amino acid sequence having or having at least 85%, 86%, 87%, 88%, 89%, 90%, 91%, 92%, 93%, 94%, 95%, 96%, 97%, 98%, or 99% identity to SEQ ID NO: 18. In some embodiments, the bispecific antibody or antigen binding fragment thereof is a bispecific antigen binding fragment comprising an amino acid sequence set forth in SEQ ID NO: 18.

In some embodiments, a dose of the bispecific antibody or antigen binding fragment thereof is administered to the patient at 0.01, 0.04, 0.15, 0.5, 1.5, 3.0, 4.5 or 7.0 mg/kg. In some embodiments, a dose of the bispecific antibody or antigen binding fragment thereof comprises 200 mg of the bispecific antibody or antigen binding fragment thereof.

In some embodiments, the cancer is selected from the group consisting of Hodgkin lymphoma, non-Hodgkin lymphoma, peripheral T-cell lymphoma, cutaneous T cell lymphoma, anaplastic large-cell lymphoma, CD30+B-cell lymphoma, multiple myeloma, and leukemia. In some embodiments, the cancer is Hodgkin lymphoma. In some embodiments, the cancer is peripheral T-cell lymphoma. In some embodiments, the patient has relapsed after treatment with or is refractory to an anti-CD30 antibody. In some embodiments, the anti-CD30 antibody is brentuximab vedotin. In some embodiments, the patient has experienced disease progression after treatment with autologous stem cell transplant or chimeric antigen receptor T-cell therapy (CAR-T).

In some embodiments, the patient is administered 1×10⁸ to 1×10¹⁰ NK cells per dose of NK cells. In some embodiments, the patient is administered 1×10⁹ to 8×10⁹ NK cells per dose of NK cells. In some embodiments, the patient is administered 4×10⁸, 1×10⁹, 4×10⁹, 8×10⁹ NK, or 1.6×10¹⁰ cells per dose of NK cells.

In some embodiments, the patient is subjected to lymphodepleting chemotherapy prior to treatment. In some embodiments, the lymphodepleting chemotherapy is non-myeloablative chemotherapy. In some embodiments, the lymphodepleting chemotherapy comprises treatment with at least one of cyclophosphamide and fludarabine. In some embodiments, the lymphodepleting chemotherapy comprises treatment with cyclophosphamide and fludarabine. In some embodiments, the cyclophosphamide is administered between 100 and 500 mg/m²/day. In some embodiments, the cyclophosphamide is administered at 250 mg/m²/day. In some embodiments, the cyclophosphamide is administered at 500 mg/m²/day. In some embodiments, the fludarabine is administered between 10 and 50 mg/m²/day. In some embodiments, the fludarabine is administered at 30 mg/m²/day.

In some embodiments, the method further comprises administering IL-2 to the patient. In some embodiments, the patient is administered 1×10⁶ IU/m² of IL-2 per dose. In some embodiments, the patient is administered 1 million or 6 million IU of IL-2 per dose. In some embodiments, administration of IL-2 occurs within 1-4 hours of administration of the NK cells.

In some embodiments, the administration of a dose of the first pharmaceutical composition comprising the NK cell and a dose of the second pharmaceutical composition comprising the bispecific antibody or antigen binding fragment thereof occurs weekly. In some embodiments, the NK cells and the first pharmaceutical composition comprising the NK cell and the second pharmaceutical composition comprising the bispecific antibody or antigen binding fragment thereof are administered weekly for 4 to 8 weeks. In some embodiments, the administration of the first pharmaceutical composition comprising the NK cell occurs weekly for three weeks and the second pharmaceutical composition comprising the bispecific antibody or antigen binding fragment thereof occurs weekly for six weeks. In some embodiments, the administration of the first pharmaceutical composition comprising the NK cell occurs every other week for six weeks and the second pharmaceutical composition comprising the bispecific antibody or antigen binding fragment thereof occurs weekly for six weeks.

Also provided herein are methods for treating a patient suffering from a CD30+ cancer, the method comprising: administering to the patient a first cycle of treatment comprising any one of the methods of treatment described herein; and administering to the patient a second cycle of treatment comprising the method of any one of the methods of treatment described herein, wherein the first cycle of treatment and the second cycle of treatment are the same or different.

In some embodiments, the method further comprises administering to the patient a third cycle of treatment comprising the method of any one of the methods described herein.

In some embodiments, the method comprises a treatment break of at least two weeks between cycles.

In some embodiments, the treatment continues until the CD30+ cancer progresses, or until the doses are discontinued due to the patient's intolerance of the NK cell, the bispecific antibody or antigen binding fragment thereof, or both, or until the patient experiences toxicity the NK cells, the bispecific antibody or antigen binding fragment thereof, or both.

In some embodiments, the NK cells are not genetically modified.

In some embodiments, at least 70% of the NK cells are CD56+ and CD16+. In some embodiments, at least 85% of the NK cells are CD56+ and CD3−. In some embodiments, 1% or less of the NK cells are CD3+, 1% or less of the NK cells are CD19+ and 1% or less of the NK cells are CD14+.

Also provided herein are pharmaceutical compositions comprising: (a) a natural killer cell (NK cell) comprising a KIR-B haplotype and expression of a CD16 molecule; and (b) a bispecific antibody or antigen binding fragment thereof comprising a first binding domain that specifically binds to CD16 (FcγRIII) and a second binding domain that specifically binds to CD30, wherein the first binding domain that specifically binds to CD16 comprises: a light chain variable domain (VL_CD16A) comprising a light chain complementarity determining region 1 (CDRL1) comprising SEQ ID NO: 9, a light chain complementarity determining region 2 (CDRL2) comprising SEQ ID NO: 10: a light chain complementarity determining region 3 (CDRL3) comprising SEQ ID NO: 11; and a heavy chain variable domain (VH_CD16A) comprising a heavy chain complementarity determining region 1 (CDRH1 comprising SEQ ID NO: 6; a heavy chain complementarity determining region 2 (CDRH2) comprising SEQ ID NO: 7; and a heavy chain complementarity determining region 3 (CDRH3) comprising SEQ ID NO: 8; and wherein the second binding domain that specifically binds to CD30 comprises: a light chain variable domain (VL_CD30) comprising a light chain complementarity determining region 1 (CDRL1) comprising SEQ ID NO: 15, a light chain complementarity determining region 2 (CDRL2) comprising SEQ ID NO: 16: a light chain complementarity determining region 3 (CDRL3) comprising SEQ ID NO: 17; and a heavy chain variable domain (VH_CD30) comprising a heavy chain complementarity determining region 1 (CDRH1 comprising SEQ ID NO: 12: a heavy chain complementarity determining region 2 (CDRH2) comprising SEQ ID NO: 13; and a heavy chain complementarity determining region 3 (CDRH3) comprising SEQ ID NO: 14.

In some embodiments, the CD16 molecule is a CD16A molecule. In some embodiments, the CD16 molecule comprises a V/V polymorphism at F158. In some embodiments, the bispecific antibody that specifically binds to CD16 specifically binds to CD16A.

In some embodiments, the first binding domain that specifically binds to CD16 comprises a light chain variable (V_{L}) region comprising SEQ ID NO: 20 and a heavy chain variable (V_{H}) region comprising SEQ ID NO: 19. In some embodiments, the first binding domain that specifically binds to CD16 comprises a V_{L} region comprising an amino acid sequence having or having at least 85%, 86%, 87%, 88%, 89%, 90%, 91%, 92%, 93%, 94%, 95%, 96%, 97%, 98%, or 99% identity to SEQ ID NO: 20 and a Vu region comprising an amino acid sequence having or having at least 85%, 86%, 87%, 88%, 89%, 90%, 91%, 92%, 93%, 94%, 95%, 96%, 97%, 98%, or 99% identity to SEQ ID NO: 19. In some embodiments, the second binding domain that specifically binds to CD30 comprises a light chain variable (V_{L}) region comprising SEQ ID NO: 22 and a heavy chain variable (V_{H}) region comprising SEQ ID NO: 21. In some embodiments, the second binding domain that specifically binds to CD30 comprises a V_{L} region comprising an amino acid sequence having or having at least 85%, 86%, 87%, 88%, 89%, 90%, 91%, 92%, 93%, 94%, 95%, 96%, 97%, 98%, or 99% identity to SEQ ID NO: 22 and a Vu region comprising an amino acid sequence having or having at least 85%, 86%, 87%, 88%, 89%, 90%, 91%, 92%, 93%, 94%, 95%, 96%, 97%, 98%, or 99% identity to SEQ ID NO: 21.

In some embodiments, the bispecific antibody or antigen binding fragment thereof is a bispecific antigen binding fragment; and the variable domains of the bispecific antigen binding fragment are linked by peptide linkers L1, L2, and L3 from the N-terminus to the C-terminus in the order: VH_CD30-L1-VL_CD16A-L2-VH_CD16A-L3-VL_CD30. In some embodiments the bispecific antibody or antigen binding fragment thereof is a bispecific antigen binding fragment; and the variable domains of the bispecific antigen binding fragment are linked by peptide linkers L1, L2, and L3 from the N-terminus to the C-terminus in the order: VH_CD16A-L1-VL_CD30-L2-VH_CD30-L3-VL_CD16A.

In some embodiments, each of peptide linkers L1, L2, and L3 consist of no more than 12 amino acid residues. In some embodiments, linker L2 of the antibody construct consists of between 3 and 9 amino acid residues, inclusive.

In some embodiments, the bispecific antibody or antigen binding fragment thereof is a comprises an amino acid sequence having or having at least 85%, 86%, 87%, 88%, 89%, 90%, 91%, 92%, 93%, 94%, 95%, 96%, 97%, 98%, or 99% identity to SEQ ID NO: 18. In some embodiments, the bispecific antibody or antigen binding fragment thereof is a bispecific antigen binding fragment comprising an amino acid sequence set forth in SEQ ID NO: 18.

In some embodiments, the NK cell is a cord blood-derived NK cell.

In some embodiments, the cord blood-derived NK cell has been produced by a method comprising: (a) providing a sample of cord blood cells comprising natural killer cells; (b) depleting the cells of CD3 (+) cells; (b) expanding the natural killer cells by culturing the seed cells with a first plurality of cells from an inactivated CD4 (+) T cell line in a medium comprising: a T-cell stimulating antibody selected from the group consisting of OKT3, UCHT1, HTa, or a combination thereof; and IL-2, to produce the cord blood-derived natural killer cells.

In some embodiments, the inactivated CD4 (+) T cell line expresses at least one gene selected from the group consisting of a 4-1BBL gene, a membrane-bound IL-21 (mbIL-21) gene, an OX40L gene, and a mouse TNF-α gene. In some embodiments, the inactivated CD4 (+) T cell line expresses a 4-1BBL gene, a membrane-bound IL-21 (mbIL-21) gene, and a mouse TNF-α gene.

In some embodiments, the first binding domain that specifically binds to CD16 of the bispecific antibody or antigen binding fragment thereof is bound to the CD16 molecule of the NK cell.

In some embodiments, the pharmaceutical composition further comprises (a) human albumin; (b) dextran; (c) glucose; (d) DMSO; and (e) a buffer. In some embodiments, the pharmaceutical composition comprises from 30 to 50 mg/mL human albumin. In some embodiments, the pharmaceutical composition comprises 50 mg/mL human albumin. In some embodiments, the pharmaceutical composition comprises 20 to 30 mg/mL dextran. In some embodiments, the pharmaceutical composition comprises 25 mg/mL dextran. In some embodiments, the dextran is Dextran 40. In some embodiments, the pharmaceutical composition comprises from 12 to 15 mg/mL glucose. In some embodiments, the pharmaceutical composition comprises 12.5 mg/mL glucose. In some embodiments, the pharmaceutical composition comprises less than 27.5 g/L glucose. In some embodiments, the pharmaceutical composition comprises from 50 to 60 ml/mL DMSO. In some embodiments, the pharmaceutical composition comprises 55 mg/mL DMSO. In some embodiments, the pharmaceutical composition comprises 40 to 60% v/v buffer. In some embodiments, the buffer is phosphate buffered saline. In some embodiments, the pharmaceutical composition comprises: (a) about 40 mg/mL human albumin; (b) about 25 mg/mL Dextran 40; (c) about 12.5 mg/mL glucose; (d) about 55 mg/mL DMSO; and (e) about 0.5 mL/mL phosphate buffered saline. In some embodiments, the pharmaceutical composition further comprises 0.5 mL/mL water. In some embodiments, the pharmaceutical composition further comprises a pharmaceutically acceptable excipient.

Also provided herein are frozen vial(s) comprising a pharmaceutical composition described herein.

Also provided herein are methods for treating a patient suffering from a CD30+ cancer, the method comprising administering a pharmaceutical composition described herein.

Also provided herein are pharmaceutical compositions comprising: (a) a natural killer cell (NK cell) comprising a KIR-B haplotype and expression of a CD16 molecule; and (b) a bispecific antibody or antigen binding fragment thereof comprising a first binding domain that specifically binds to CD16 (FcγRIII) and a second binding domain that specifically binds to CD30, wherein the first binding domain that specifically binds to CD16 comprises: a light chain variable domain (VL_CD16A) comprising a light chain complementarity determining region 1 (CDRL1) comprising SEQ ID NO: 9, a light chain complementarity determining region 2 (CDRL2) comprising SEQ ID NO: 10: a light chain complementarity determining region 3 (CDRL3) comprising SEQ ID NO: 11; and a heavy chain variable domain (VH_CD16A) comprising a heavy chain complementarity determining region 1 (CDRH1 comprising SEQ ID NO: 6; a heavy chain complementarity determining region 2 (CDRH2) comprising SEQ ID NO: 7; and a heavy chain complementarity determining region 3 (CDRH3) comprising SEQ ID NO: 8; and wherein the second binding domain that specifically binds to CD30 comprises: a light chain variable domain (VL_CD30) comprising a light chain complementarity determining region 1 (CDRL1) comprising SEQ ID NO: 15, a light chain complementarity determining region 2 (CDRL2) comprising SEQ ID NO: 16: a light chain complementarity determining region 3 (CDRL3) comprising SEQ ID NO: 17; and a heavy chain variable domain (VH_CD30) comprising a heavy chain complementarity determining region 1 (CDRH1 comprising SEQ ID NO: 12: a heavy chain complementarity determining region 2 (CDRH2) comprising SEQ ID NO: 13; and a heavy chain complementarity determining region 3 (CDRH3) comprising SEQ ID NO: 14.

In some embodiments, the CD16 molecule is a CD16A molecule.

In some embodiments the CD16 molecule comprises a V/V polymorphism at F158.

In some embodiments, the bispecific antibody that specifically binds to CD16 specifically binds to CD16A.

In some embodiments, the first binding domain that specifically binds to CD16 comprises a light chain variable (V_{L}) region comprising SEQ ID NO: 20 and a heavy chain variable (V_{H}) region comprising SEQ ID NO: 19.

In some embodiments, the first binding domain that specifically binds to CD16 comprises a V_{L} region comprising an amino acid sequence having or having at least 85%, 86%, 87%, 88%, 89%, 90%, 91%, 92%, 93%, 94%, 95%, 96%, 97%, 98%, or 99% identity to SEQ ID NO: 20 and a V_{H} region comprising an amino acid sequence having or having at least 85%, 86%, 87%, 88%, 89%, 90%, 91%, 92%, 93%, 94%, 95%, 96%, 97%, 98%, or 99% identity to SEQ ID NO: 19.

In some embodiments, the second binding domain that specifically binds to CD30 comprises a light chain variable (V_{L}) region comprising SEQ ID NO: 22 and a heavy chain variable (V_{H}) region comprising SEQ ID NO: 21.

In some embodiments the second binding domain that specifically binds to CD30 comprises a V_{L} region comprising an amino acid sequence having or having at least 85%, 86%, 87%, 88%, 89%, 90%, 91%, 92%, 93%, 94%, 95%, 96%, 97%, 98%, or 99% identity to SEQ ID NO: 22 and a V_{H} region comprising an amino acid sequence having or having at least 85%, 86%, 87%, 88%, 89%, 90%, 91%, 92%, 93%, 94%, 95%, 96%, 97%, 98%, or 99% identity to SEQ ID NO: 21.

In some embodiments, the bispecific antibody or antigen binding fragment thereof is a bispecific antigen binding fragment; and the variable domains of the bispecific antigen binding fragment are linked by peptide linkers L1, L2, and L3 from the N-terminus to the C-terminus in the order: V_{H}_CD30-L1-V_{L}_CD16A-L2-V_{H}_CD16A-L3-V_{L}_CD30. In some embodiments each of peptide linkers L1, L2, and L3 consist of no more than 12 amino acid residues. In some embodiments, linker L2 of the antibody construct consists of between 3 and 9 amino acid residues, inclusive.

In some embodiments, the bispecific antibody or antigen binding fragment thereof is a bispecific antigen binding fragment; and the variable domains of the bispecific antigen binding fragment are linked by peptide linkers L1, L2, and L3 from the N-terminus to the C-terminus in the order: V_{H}_CD16A-L1-V_{L}_CD30-L2-V_{H}_CD30-L3-V_{L}_CD16A. In some embodiments each of peptide linkers L1, L2, and L3 consist of no more than 12 amino acid residues. In some embodiments, linker L2 of the antibody construct consists of between 3 and 9 amino acid residues, inclusive.

In some embodiments the bispecific antibody or antigen binding fragment thereof is a comprises an amino acid sequence having or having at least 85%, 86%, 87%, 88%, 89%, 90%, 91%, 92%, 93%, 94%, 95%, 96%, 97%, 98%, or 99% identity to SEQ ID NO: 18.

In some embodiments the bispecific antibody or antigen binding fragment thereof is a bispecific antigen binding fragment comprising an amino acid sequence set forth in SEQ ID NO: 18.

In some embodiments the NK cell is a cord blood-derived NK cell.

In some embodiments the cord blood-derived NK cell has been produced by a method comprising: (a) providing a sample of cord blood cells comprising natural killer cells; (b) depleting the cells of CD3 (+) cells; (b) expanding the natural killer cells by culturing the seed cells with a first plurality of cells from an inactivated CD4 (+) T cell line in a medium comprising: a T-cell stimulating antibody selected from the group consisting of OKT3, UCHT1, HTa, or a combination thereof; and IL-2, to produce the cord blood-derived natural killer cells.

In some embodiments the inactivated CD4 (+) T cell line expresses at least one gene selected from the group consisting of a 4-1BBL gene, a membrane-bound IL-21 (mbIL-21) gene, an OX40L gene, and a mouse TNF-α gene.

In some embodiments the inactivated CD4 (+) T cell line expresses a 4-1BBL gene, a membrane-bound IL-21 (mbIL-21) gene, and a mouse TNF-α gene.

In some embodiments the first binding domain that specifically binds to CD16 of the bispecific antibody or antigen binding fragment thereof is bound to the CD16 molecule of the NK cell.

In some embodiments the pharmaceutical composition further comprises (a) human albumin; (b) dextran; (c) glucose; (d) DMSO; and (e) a buffer. In some embodiments, the pharmaceutical composition comprises from 30 to 50 mg/mL human albumin. In some embodiments, the pharmaceutical composition comprises 50 mg/mL human albumin. In some embodiments, the pharmaceutical composition comprises 20 to 30 mg/mL dextran. In some embodiments, the pharmaceutical composition comprises 25 mg/mL dextran. In some embodiments, the dextran is Dextran 40. In some embodiments, the pharmaceutical composition comprises from 12 to 15 mg/mL glucose. In some embodiments, the pharmaceutical composition comprises 12.5 mg/mL glucose. In some embodiments, the pharmaceutical composition comprises less than 27.5 g/L glucose. In some embodiments, the pharmaceutical composition comprises from 50 to 60 ml/mL DMSO. In some embodiments, the pharmaceutical composition comprises 55 mg/mL DMSO. In some embodiments, the pharmaceutical composition comprises 40 to 60% v/v buffer. In some embodiments, the buffer is phosphate buffered saline. In some embodiments, the pharmaceutical composition comprises: (a) about 40 mg/mL human albumin; (b) about 25 mg/mL Dextran 40; (c) about 12.5 mg/mL glucose; (d) about 55 mg/mL DMSO; and (e) about 0.5 mL/mL phosphate buffered saline. In some embodiments, the pharmaceutical composition comprises 0.5 mL/mL water.

In some embodiments, the pharmaceutical composition comprises a pharmaceutically acceptable excipient.

Also described herein are frozen vials comprising any of the pharmaceutical compositions described herein.

Also described herein are methods for treating a patient suffering from a CD30+ cancer comprising administering any of the pharmaceutical compositions described herein.

Also described herein are methods for treating a patient suffering from a CD30+ cancer comprising: administering a first pharmaceutical composition comprising a natural killer cell (NK cell) comprising a KIR-B haplotype and expression of a CD16 molecule; and administering a second pharmaceutical composition comprising a bispecific antibody or antigen binding fragment thereof comprising a first binding domain that specifically binds to CD16 (FcγRIII) and a second binding domain that specifically binds to CD30, wherein the first binding domain that specifically binds to CD16 comprises: a light chain variable domain (VL_CD16A) comprising a light chain complementarity determining region 1 (CDRL1) comprising SEQ ID NO: 9, a light chain complementarity determining region 2 (CDRL2) comprising SEQ ID NO: 10; a light chain complementarity determining region 3 (CDRL3) comprising SEQ ID NO: 11; and a heavy chain variable domain (VH_CD16A) comprising a heavy chain complementarity determining region 1 (CDRH1 comprising SEQ ID NO: 6: a heavy chain complementarity determining region 2 (CDRH2) comprising SEQ ID NO: 7; and a heavy chain complementarity determining region 3 (CDRH3) comprising SEQ ID NO: 8; and wherein the second binding domain that specifically binds to CD30 comprises: a light chain variable domain (VL_CD30) comprising a light chain complementarity determining region 1 (CDRL1) comprising SEQ ID NO: 15, a light chain complementarity determining region 2 (CDRL2) comprising SEQ ID NO: 16: a light chain complementarity determining region 3 (CDRL3) comprising SEQ ID NO: 17; and a heavy chain variable domain (VH_CD30) comprising a heavy chain complementarity determining region 1 (CDRH1 comprising SEQ ID NO: 12: a heavy chain complementarity determining region 2 (CDRH2) comprising SEQ ID NO: 13; and a heavy chain complementarity determining region 3 (CDRH3) comprising SEQ ID NO: 14.

In some embodiments, the NK cell is a cord blood-derived NK cell.

In some embodiments, the cord blood-derived NK cell has been produced by a method comprising: (a) providing a sample of cord blood cells comprising natural killer cells; (b) depleting the cells of CD3 (+) cells; (b) expanding the natural killer cells by culturing the seed cells with a first plurality of cells from an inactivated CD4 (+) T cell line in a medium comprising: a T-cell stimulating antibody selected from the group consisting of OKT3, UCHT1, HTa, or a combination thereof; and IL-2, to produce the cord blood-derived natural killer cells.

In some embodiments, the inactivated CD4 (+) T cell line expresses at least one gene selected from the group consisting of a 4-1BBL gene, a membrane-bound IL-21 (mbIL-21) gene, an OX40L gene, and a mouse TNF-α gene.

In some embodiments, the inactivated CD4 (+) T cell line expresses a 4-1BBL gene, a membrane-bound IL-21 (mbIL-21) gene, and a mouse TNF-α gene.

In some embodiments, the CD16 molecule is a CD16A molecule.

In some embodiments, the CD16 molecule comprises a V/V polymorphism at F158.

In some embodiments, the first binding domain that specifically binds to CD16 specifically binds to CD16A.

In some embodiments, the first pharmaceutical composition further comprises: (a) human albumin; (b) dextran; (c) glucose; (d) DMSO; and (e) a buffer. In some embodiments, the first pharmaceutical composition comprises from 30 to 50 mg/mL human albumin. In some embodiments, the first pharmaceutical composition comprises 50 mg/mL human albumin. In some embodiments, the first pharmaceutical composition comprises 20 to 30 mg/mL dextran. In some embodiments, the first pharmaceutical composition comprises 25 mg/mL dextran. In some embodiments, the dextran is Dextran 40. In some embodiments, the first pharmaceutical composition comprises from 12 to 15 mg/mL glucose. In some embodiments, the first pharmaceutical composition comprises 12.5 mg/mL glucose. In some embodiments, the first pharmaceutical composition comprises less than 27.5 g/L glucose. In some embodiments, the first pharmaceutical composition comprises from 50 to 60 ml/mL DMSO. In some embodiments, the first pharmaceutical composition comprises 55 mg/mL DMSO. In some embodiments, the first pharmaceutical composition comprises 40 to 60% v/v buffer. In some embodiments, the buffer is phosphate buffered saline. In some embodiments, the first pharmaceutical composition further comprises: (a) about 40 mg/mL human albumin; (b) about 25 mg/mL Dextran 40; (c) about 12.5 mg/mL glucose; (d) about 55 mg/mL DMSO; and (e) about 0.5 mL/mL phosphate buffered saline. In some embodiments, the pharmaceutical composition further comprises 0.5 mL/mL water.

In some embodiments, the first pharmaceutical composition further comprises a pharmaceutically acceptable excipient.

In some embodiments, the first binding domain that specifically binds to CD16 comprises a light chain variable (V_{L}) region comprising SEQ ID NO: 20 and a heavy chain variable (V_{H}) region comprising SEQ ID NO: 19.

In some embodiments, the first binding domain that specifically binds to CD16 comprises a V_{L} region comprising an amino acid sequence having or having at least 85%, 86%, 87%, 88%, 89%, 90%, 91%, 92%, 93%, 94%, 95%, 96%, 97%, 98%, or 99% identity to SEQ ID NO: 20 and a V_{H} region comprising an amino acid sequence having or having at least 85%, 86%, 87%, 88%, 89%, 90%, 91%, 92%, 93%, 94%, 95%, 96%, 97%, 98%, or 99% identity to SEQ ID NO: 19.

In some embodiments, the second binding domain that specifically binds to CD30 comprises a light chain variable (V_{L}) region comprising SEQ ID NO: 22 and a heavy chain variable (V_{H}) region comprising SEQ ID NO: 21.

In some embodiments, the second binding domain that specifically binds to CD30 comprises a V_{L} region comprising an amino acid sequence having or having at least 85%, 86%, 87%, 88%, 89%, 90%, 91%, 92%, 93%, 94%, 95%, 96%, 97%, 98%, or 99% identity to SEQ ID NO: 22 and a V_{H} region comprising an amino acid sequence having or having at least 85%, 86%, 87%, 88%, 89%, 90%, 91%, 92%, 93%, 94%, 95%, 96%, 97%, 98%, or 99% identity to SEQ ID NO: 21.

In some embodiments, the bispecific antibody or antigen binding fragment thereof is a bispecific antigen binding fragment; and the variable domains of the bispecific antigen binding fragment are linked by peptide linkers L1, L2, and L3 from the N-terminus to the C-terminus in the order: VH_CD30-L1-VL_CD16A-L2-VH_CD16A-L3-VL_CD30. In some embodiments, the bispecific antibody or antigen binding fragment thereof is a bispecific antigen binding fragment; and the variable domains of the bispecific antigen binding fragment are linked by peptide linkers L1, L2, and L3 from the N-terminus to the C-terminus in the order: VH_CD16A-L1-VL_CD30-L2-VH_CD30-L3-VL_CD16A. In some embodiments, each of peptide linkers L1, L2, and L3 consist of no more than 12 amino acid residues. In some embodiments, linker L2 of the antibody construct consists of between 3 and 9 amino acid residues, inclusive.

In some embodiments, the bispecific antibody or antigen binding fragment thereof is a comprises an amino acid sequence having or having at least 85%, 86%, 87%, 88%, 89%, 90%, 91%, 92%, 93%, 94%, 95%, 96%, 97%, 98%, or 99% identity to SEQ ID NO: 18.

In some embodiments, the bispecific antibody or antigen binding fragment thereof is a bispecific antigen binding fragment comprising an amino acid sequence set forth in SEQ ID NO: 18.

Unless otherwise defined, all technical and scientific terms used herein have the same meaning as commonly understood by one of ordinary skill in the art to which this invention belongs. Methods and materials are described herein for use in the present invention: other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows an exemplary embodiment of a method for NK cell expansion and stimulation.

FIG. 2 shows that cord blood-derived NK cells (CB-NK) have an approximately ten-fold greater ability to expand in culture than peripheral blood-derived NK cells (PB-NK) in preclinical studies.

FIG. 3 shows that expression of tumor-engaging NK activating immune receptors was higher and more consistent in cord blood-derived drug product compared to that generated from peripheral blood.

FIG. 4 shows phenotypes of expanded and stimulated population of NK cells.

FIG. 5 shows key steps in the manufacture of the AB-101 drug product, which is an example of a cord blood-derived and expanded population of NK cells.

FIG. 6 shows the purity of AB-101 (n=9).

FIG. 7 shows purity of CD3 depleted cells, MCB and DP manufactured in GMP conditions.

FIG. 8 shows expression of NK cell receptors on CD3 depleted cells, MCB and DP manufactured in GMP conditions.

FIG. 9 shows NK purity (CD56+/CD3−) by flow cytometry.

FIG. 10 shows CD38+ expression of expanded NK cells from three different cord blood donors.

FIG. 11 shows CD38+ mean fluorescence intensity of CD38+NK cells from three different cord blood donors.

FIG. 12 shows differential surface protein expression of starting NK cell source compared to AB-101 cells.

FIG. 13 shows cytotoxic activity of AFM13 and AB-101 NK cells from MCB2, in 4 hour calcein-release assays on KARPAS-299 target cells at decreasing effector-to-target (E:T) ratios starting at 10:1 followed by two-fold serial dilutions.

FIG. 14 shows cytotoxic activity of AFM13 and AB-101 NK cells from MCB1, in 4 hour calcein-release assays on KARPAS-299 target cells at decreasing effector-to-target (E:T) ratios starting at 10:1 followed by two-fold serial dilutions.

FIG. 15 shows a bar graph of cytotoxic activity of AFM13 and AB-101 NK cells from MCB1 (right, AB-101 MCB1) and of MCB2 (left, AB-101 MCB2), in 4 hour calcein-release assays on KARPAS-299 target cells at an effector-to-target (E:T) ratio of 5:1.

FIG. 16 shows retention of bound AFM13 on preloaded cryopreserved AB-101 cells from MCB2 after thawing, wherein filled histograms represent anti-AFM13 (rat anti-AFM13 antibody)+secondary antibody (goat anti-rat FITC antibody) and open histograms represent secondary antibody only. From top to bottom: non-preloaded: non-preloaded+fresh excess AFM: AFM-preloaded: AFM-preloaded+fresh excess AFM.

FIG. 17 shows retention of bound AFM13 on preloaded cryopreserved AB-101 cells from MCB1 after thawing, wherein filled histograms represent anti-AFM13 (rat anti-AFM13 antibody)+secondary antibody (goat anti-rat FITC antibody) and open histograms represent secondary antibody only. From top to bottom: non-preloaded: non-preloaded+fresh excess AFM: AFM-preloaded: AFM-preloaded+fresh excess AFM.

FIG. 18 shows fluorescence intensity on CD16 expression on preloaded AB-101 cells (left: MCB2, right: MCB1). The various conditions show uniform expression of CD16 on AB-101 cells. From top to bottom: non-preloaded: non-preloaded+fresh excess AFM: AFM-preloaded; AFM-preloaded+fresh excess AFM.

FIG. 19 shows NK fratricide (NK-NK cell lysis) by AFM13 on AB-101 NK cells from MCB2, in 4 hour calcein-release assays at an effector-to-target (E:T) ratio of 1:1.

FIG. 20 shows NK fratricide (NK-NK cell lysis) by AFM13 on AB-101 NK cells from MCB1, in 4 hour calcein-release assays at an effector-to-target (E:T) ratio of 1:1.

FIG. 21 shows up-regulation of CD107a in response to Karpas-299 target cells and AFM13, wherein the AB-101 NK cells from MCB2 were co-cultured with and without the target cells at a 1:1 cell ratio, wherein the % CD107a+ NK cells was determined by flow cytometry.

FIG. 22 shows up-regulation of CD107a in response to Karpas-299 target cells and AFM13, wherein the AB-101 NK cells from MCB1 were co-cultured with and without the target cells at a 1:1 cell ratio, wherein the % CD107a+ NK cells was determined by flow cytometry.

FIG. 23 shows increased production of intracellular IFNγ in response to Karpas-299 target cells and AFM13, wherein the AB-101 NK cells from MCB2 were co-cultured with and without the target cells at a 1:1 cell ratio, wherein the % IFNγ+NK cells was determined by flow cytometry.

FIG. 24 shows increased production of intracellular IFNγ in response to Karpas-299 target cells and AFM13, wherein the AB-101 NK cells from MCB1 were co-cultured with and without the target cells at a 1:1 cell ratio, wherein the % IFNγ+NK cells was determined by flow cytometry.

FIG. 25 shows viability analysis of cryopreserved AFM13-preloaded AB-101 NK cells, wherein the efficacy of AFM13-preloaded or empty AB-101 NK cells were evaluated on MDA-MB-231-Luc cells in an intraperitoneal xenograft tumor model in female hIL15-NOG mice.

FIG. 26 shows the experimental design for an in vivo efficacy study combining AFM13 and AB-101 in the Karpas-299/Luc Human Tumor Xenograft Model.

FIG. 27 shows results of an in vivo efficacy study combining AFM13 and AB-101 in the Karpas-299/Luc Human Tumor Xenograft Model.

### DETAILED DESCRIPTION

Provided herein are, among other things, pharmaceutical compositions comprising NK cell(s), e.g., as described herein, and multispecific engager(s), e.g., as described herein as well as frozen vial(s) comprising the pharmaceutical composition(s), and methods for treating patients with the pharmaceutical composition(s).

### I. Expansion and Stimulation of Natural Killer Cells

In some embodiments, natural killer cells are expanded and stimulated, e.g., by culturing and stimulation with feeder cells.

NK cells can be expanded and stimulated as described, for example, in US 2020/0108096 or WO 2020/101361, both of which are incorporated herein by reference in their entirety. Briefly, the source cells can be cultured on modified HuT-78 (ATCC® TIB-161™) cells that have been engineered to express 4-1BBL, membrane bound IL-21, and a mutant TNFα as described in US 2020/0108096.

Suitable NK cells can also be expanded and stimulated as described herein.

In some embodiments, NK cells are expanded and stimulated by a method comprising: (a) providing NK cells, e.g., a composition comprising NK cells, e.g., CD3 (+) depleted NK cells; and (b) culturing in a medium comprising feeder cells and/or stimulation factors, thereby producing a population of expanded and stimulated NK cells.

### A. Natural Killer Cell Sources

In some embodiments, the NK cell source is selected from the group consisting of peripheral blood, peripheral blood lymphocytes (PBLs), peripheral blood mononuclear cells (PBMCs), bone marrow; umbilical cord blood (cord blood), isolated NK cells, NK cells derived from induced pluripotent stem cells, NK cells derived from embryonic stem cells, and combinations thereof.

In some embodiments, the NK cell source is a single unit of cord blood.

In some embodiments, the natural killer cell source, e.g., single unit of cord blood, comprises from or from about 1×10⁷ to or to about 1×10⁹ total nucleated cells. In some embodiments, the natural killer cell source, e.g., single unit of cord blood, comprises from or from about 1×10⁸ to or to about 1.5×10⁸ total nucleated cells. In some embodiments, the natural killer cell source, e.g., single unit of cord blood, comprises 1×10⁸ total nucleated cells. In some embodiments, the natural killer cell source, e.g., single unit of cord blood, comprises about 1×10⁸ total nucleated cells. In some embodiments, the natural killer cell source, e.g., single unit of cord blood, comprises 1×10⁹ total nucleated cells. In some embodiments, the natural killer cell source, e.g., single unit of cord blood, comprises about 1×10⁹ total nucleated cells.

In some embodiments, the NK cell source, e.g., the cord blood unit, comprises from about 20% to about 80% CD16+ cells. In some embodiments, the NK cell source, e.g., the cord blood unit, comprises from or from about 20% to or to about 80%, from about 20% to or to about 70%, from about 20% to or to about 60%, from about 20% to or to about 50%, from about 20% to or to about 40%, from about 20% to or to about 30%, from about 30% to or to about 80%, from about 30% to or to about 70%, from about 30% to or to about 60%, from about 30% to or to about 50%, from about 30% to or to about 40%, from about 40% to or to about 80%, from about 40% to or to about 70%, from about 40% to or to about 60%, from about 40% to or to about 50%, from about 50% to or to about 80%, from about 50% to or to about 70%, from about 50% to or to about 60%, from about 60% to or to about 80%, from about 60% to or to about 70%, or from about 70% to or to about 80% CD16+ cells. In some embodiments, the NK cell source, e.g., the cord blood unit, comprises less than or equal to 80% CD16+ cells. Alternately, some NK cell sources may comprise CD16+ cells at a concentration of greater than 80%.

In some embodiments, the NK cell source, e.g., the cord blood unit, comprises less than or equal to 40%, e.g., less than or equal to 30%, e.g., less than or equal to 20%, e.g., less than or equal to 10%, e.g., less than or equal to 5% MLG2A+ cells.

In some embodiments, the NK cell source, e.g., the cord blood unit, comprises less than or equal to 40%, e.g., less than or equal to 30%, e.g., less than or equal to 20%, e.g., less than or equal to 10%, e.g., less than or equal to 5% NKG2C+ cells.

In some embodiments, the NK cell source, e.g., the cord blood unit, comprises less than or equal to 40%, e.g., less than or equal to 30%, e.g., less than or equal to 20%, e.g., less than or equal to 10%, e.g., less than or equal to 5% NKG2D+ cells.

In some embodiments, the NK cell source, e.g., the cord blood unit, comprises less than or equal to 40%, e.g., less than or equal to 30%, e.g., less than or equal to 20%, e.g., less than or equal to 10%, e.g., less than or equal to 5% NKp46+ cells.

In some embodiments, the NK cell source, e.g., the cord blood unit, comprises less than or equal to 40%, e.g., less than or equal to 30%, e.g., less than or equal to 20%, e.g., less than or equal to 10%, e.g., less than or equal to 5% NKp30+ cells.

In some embodiments, the NK cell source, e.g., the cord blood unit, comprises less than or equal to 40%, e.g., less than or equal to 30%, e.g., less than or equal to 20%, e.g., less than or equal to 10%, e.g., less than or equal to 5% DNAM-1+ cells.

In some embodiments, the NK cell source, e.g., the cord blood unit, comprises less than or equal to 40%, e.g., less than or equal to 30%, e.g., less than or equal to 20%, e.g., less than or equal to 10%, e.g., less than or equal to 5% NKp44+ cells.

In some embodiments, the NK cell source, e.g., the cord blood unit, comprises less than or equal to 40%, e.g., less than or equal to 30%, e.g., less than or equal to 20%, e.g., less than or equal to 10%, e.g., less than or equal to 5% CD25+ cells.

In some embodiments, the NK cell source, e.g., the cord blood unit, comprises less than or equal to 40%, e.g., less than or equal to 30%, e.g., less than or equal to 20%, e.g., less than or equal to 10%, e.g., less than or equal to 5% CD62L+ cells.

In some embodiments, the NK cell source, e.g., the cord blood unit, comprises less than or equal to 40%, e.g., less than or equal to 30%, e.g., less than or equal to 20%, e.g., less than or equal to 10%, e.g., less than or equal to 5% CD69+ cells.

In some embodiments, the NK cell source, e.g., the cord blood unit, comprises less than or equal to 40%, e.g., less than or equal to 30%, e.g., less than or equal to 20%, e.g., less than or equal to 10%, e.g., less than or equal to 5% CXCR3+ cells.

In some embodiments, the NK cell source, e.g., the cord blood unit, comprises less than or equal to 40%, e.g., less than or equal to 30%, e.g., less than or equal to 20%, e.g., less than or equal to 10%, e.g., less than or equal to 5% CD57+ cells.

In some embodiments, NK cells in the NK cell source comprise a KIR B allele of the KIR receptor family. See. e.g., Hsu et al., “The Killer Cell Immunoglobulin-Like Receptor (KIR) Genomic Region: Gene-Order, Haplotypes and Allelic Polymorphism,” Immunological Review 190:40-52 (2002); and Pyo et al., “Different Patterns of Evolution in the Centromeric and Telomeric Regions of Group A and B Haplotypes of the Human Killer Cell Ig-like Receptor Locus,” PLOS One 5: e15115 (2010).

In some embodiments, NK cells in the NK cell source comprise the 158 V/V variant of CD16 (i.e. homozygous CD16 158V polymorphism). See, e.g., Koene et al., “FcγRIIIa-158V/F Polymorphism Influences the Binding of IgG by Natural Killer Cell FcgammaRIIIa, Independently of the FcgammaRIIIa-48L/R/H Phenotype,” Blood 90:1109-14 (1997).

In some embodiments, NK cells in the cell source comprises both the KIR B allele of the KIR receptor family and the 158 V/V variant of CD16.

In some embodiments, the NK cells in the cell source are not genetically engineered.

In some embodiments, the NK cells in the cell source do not comprise a CD16 transgene.

In some embodiments, the NK cells in the cell source do not express an exogenous CD16 protein.

In some embodiments, the NK cell source is CD3 (+) depleted. In some embodiments, the method comprises depleting the NK cell source of CD3 (+) cells. In some embodiments, depleting the NK cell source of CD3 (+) cells comprises contacting the NK cell source with a CD3 binding antibody or antigen binding fragment thereof. In some embodiments, the CD3 binding antibody or antigen binding fragment thereof is selected from the group consisting of OKT3, UCHT1, and HIT3a, and fragments thereof. In some embodiments, the CD3 binding antibody or antigen binding fragment thereof is OKT3 or an antigen binding fragment thereof. In some embodiments, the antibody or antigen binding fragment thereof is attached to a bead, e.g., a magnetic bead. In some embodiments, the depleting the composition of CD3 (+) cells comprises contacting the composition with a CD3 targeting antibody or antigen binding fragment thereof attached to a bead and removing the bead-bound CD3 (+) cells from the composition. The composition can be depleted of CD3 cells by immunomagnetic selection, for example, using a CliniMACS T cell depletion set ((LS Depletion set (162-01) Miltenyi Biotec).

In some embodiments, the NK cell source CD56+ enriched, e.g., by gating on CD56 expression.

In some embodiments, the NK cell source is both CD56+ enriched and CD3 (+) depleted, e.g., by selecting for cells with CD56+CD3− expression.

In some embodiments, the NK cell source comprises both the KIR B allele of the KIR receptor family and the 158 V/V variant of CD16 and is + enriched and CD3 (+) depleted, e.g., by selecting for cells with CD56+CD3-expression.

### B. Feeder Cells

Disclosed herein are feeder cells for the expansion of NK cells. These feeder cells advantageously allow NK cells to expand to numbers suitable for the preparation of a pharmaceutical composition as discussed herein. In some cases, the feeder cells allow the expansion of NK cells without the loss of CD16 expression, which often accompanies cell expansion on other types of feeder cells or using other methods. In some cases, the feeder cells make the expanded NK cells more permissive to freezing such that a higher proportion of NK cells remain viable after a freeze/thaw cycle or such that the cells remain viable for longer periods of time while frozen. In some cases, the feeder cells allow the NK cells to retain high levels of cytotoxicity, including ADCC, extend survival, increase persistence, and enhance or retain high levels of CD16. In some cases, the feeder cells allow the NK cells to expand without causing significant levels of exhaustion or senescence.

Feeder cells can be used to stimulate the NK cells and help them to expand more quickly, e.g., by providing substrate, growth factors, and/or cytokines.

NK cells can be stimulated using various types of feeder cells, including, but not limited to peripheral blood mononuclear cells (PBMC), Epstein-Barr virus-transformed B-lymphoblastoid cells (e.g., EBV-LCL), myelogenous leukemia cells (e.g., K562), and CD4 (+) T cells (e.g., HuT), and derivatives thereof.

In some embodiments, the feeder cells are inactivated, e.g., by γ-irradiation or mitomycin-c treatment.

Suitable feeder cells for use in the methods described herein are described, for example, in US 2020/0108096, which is hereby incorporated by reference in its entirety.

In some embodiments, the feeder cell(s) are inactivated CD4 (+) T cell(s). In some embodiments, the inactivated CD4 (+) T cell(s) are HuT-78 cells (ATCC® TIB-161™) or variants or derivatives thereof. In some embodiments, the HuT-78 derivative is H9 (ATCC® HTB-176™).

In some embodiments, the inactivated CD4 (+) T cell(s) express OX40L. In some embodiments, the inactivated CD4 (+) T cell(s) are HuT-78 cells or variants or derivatives thereof that express OX40L (SEQ ID NO: 4) or a variant thereof.

In some embodiments, the feeder cells are HuT-78 cells engineered to express at least one gene selected from the group consisting of 4-1BBL (UniProtKB P41273, SEQ ID NO: 1), membrane bound IL-21 (SEQ ID NO: 2), and mutant TNFalpha (SEQ ID NO: 3) (“eHut-78 cells”), or variants thereof.

In some embodiments, the inactivated CD4 (+) T cell(s) are HuT-78 (ATCC® TIB-161™) cells or variants or derivatives thereof that express an ortholog of OX40L, or variant thereof. In some embodiments, the feeder cells are HuT-78 cells engineered to express at least one gene selected from the group consisting of an 4-1BBL ortholog or variant thereof, a membrane bound IL-21 ortholog or variant thereof, and mutant TNFalpha ortholog, or variant thereof.

In some embodiments, the feeder cells are HuT-78 cell(s) that express OX40L (SEQ ID NO: 4) and are engineered to express 4-1BBL (SEQ ID NO: 1), membrane bound IL-21 (SEQ ID NO: 2), and mutant TNFalpha (SEQ ID NO: 3) (“eHut-78 cells”) or variants or derivatives thereof.

In some embodiments, the feeder cells are expanded, e.g., from a frozen stock, before culturing with NK cells, e.g., as described in Example 2.

### C. Stimulating Factors

NK cells can also be stimulated using one or more stimulation factors other than feeder cells, e.g., signaling factors, in addition to or in place of feeder cells.

In some embodiments, the stimulating factor, e.g., signaling factor, is a component of the culture medium, as described herein. In some embodiments, the stimulating factor, e.g., signaling factor, is a supplement to the culture medium, as described herein.

In some embodiments, the stimulation factor(s) are cytokine(s). In some embodiments, the cytokine(s) are selected from the group consisting of IL-2, IL-12, IL-15, IL-18, IL-21, IL-23, IL-27, IFN-α, IFNβ, and combinations thereof.

In some embodiments, the cytokine is IL-2.

In some embodiments, the cytokines are a combination of IL-2 and IL-15.

In some embodiments, the cytokines are a combination of IL-2, IL-15, and IL-18.

In some embodiments, the cytokines are a combination of IL-2, IL-18, and IL-21.

### D. Culturing

The NK cells can be expanded and stimulated by co-culturing an NK cell source and feeder cells and/or other stimulation factors. Suitable NK cell sources, feeder cells, and stimulation factors are described herein.

In some cases, the resulting population of expanded natural killer cells is enriched and/or sorted after expansion. In some cases, the resulting population of expanded natural killer cells is not enriched and/or sorted after expansion

Also described herein are compositions comprising the various culture compositions described herein, e.g., comprising NK cells. For example, a composition comprising a population of expanded cord blood-derived natural killer cells comprising a KIR-B haplotype and homozygous for a CD16 158V polymorphism and a plurality of engineered HuT78 cells.

Also described herein are vessels, e.g., vials, cryobags, and the like, comprising the resulting populations of expanded natural killer cells. In some cases, a plurality of vessels comprising portions of the resulting populations of expanded natural killer cells, e.g., at least 10, e.g., 20, 30, 40, 50, 60, 70, 80, 90, 100, 125, 150, 175, 200, 250, 300, 400, 500, 600, 700, 800, 900, 1000, 1100, or 1200 vessels.

Also described herein are bioreactors comprising the various culture compositions described herein, e.g., comprising NK cells. For example, a culture comprising natural killer cells from a natural killer cell source, e.g., as described herein, and feeder cells, e.g., as described herein. Also described herein are bioreactors comprising the resulting populations of expanded natural killer cells.

### 1. Culture Medium

Disclosed herein are culture media for the expansion of NK cells. These culture media advantageously allow NK cells to expand to numbers suitable for the preparation of a pharmaceutical composition as discussed herein. In some cases, the culture media allows NK cells to expand without the loss of CD16 expression that often accompanies cell expansion on other helper cells or in other media.

In some embodiments, the culture medium is a basal culture medium, optionally supplemented with additional components, e.g., as described herein.

In some embodiments, the culture medium, e.g., the basal culture medium, is a serum-free culture medium. In some embodiments, the culture medium, e.g., the basal culture medium, is a serum-free culture medium supplemented with human plasma and/or serum.

Suitable basal culture media include, but are not limited to, DMEM, RPMI 1640, MEM, DMEM/F12, SCGM (CellGenix R, 20802-0500 or 20806-0500), LGM-3™ (Lonza, CC-3211), TexMACS™ (Miltenyi Biotec, 130-097-196), ALYS™ 505NK-AC (Cell Science and Technology Institute, Inc., 01600P02), ALyS™ 505NK-EX (Cell Science and Technology Institute, Inc., 01400P10), CTS™ AIM-V™ SFM (ThermoFisher Scientific, A3830801), CTS™ OpTmizer™ (ThermoFisher Scientific, A1048501, ABS-001, StemXxVivo and combinations thereof.

The culture medium may comprise additional components, or be supplemented with additional components, such as growth factors, signaling factors, nutrients, antigen binders, and the like. Supplementation of the culture medium may occur by adding each of the additional component or components to the culture vessel either before, concurrently with, or after the medium is added to the culture vessel. The additional component or components may be added together or separately. When added separately, the additional components need not be added at the same time.

In some embodiments, the culture medium comprises plasma, e.g., human plasma. In some embodiments, the culture medium is supplemented with plasma, e.g., human plasma. In some embodiments, the plasma, e.g., human plasma, comprises an anticoagulant, e.g., trisodium citrate.

In some embodiments, the medium comprises and/or is supplemented with from or from about 0.5% to or to about 10% v/v plasma, e.g., human plasma. In some embodiments, the medium is supplemented with from or from about 0.5% to or to about 9%, from or from about 0.5% to or to about 8%, from or from about 0.5% to or to about 7%, from or from about 0.5% to or to about 6%, from or from about 0.5% to or to about 5%, from or from about 0.5% to or to about 4%, from or from about 0.5% to or to about 3%, from or from about 0.5% to or to about 2%, from or from about 0.5% to or to about 1%, from or from about 1% to or to about 10%, from or from about 1% to or to about 9%, from or from about 1% to or to about 8%, from or from about 1% to or to about 7%, from or from about 1% to or to about 6%, from or from about 1% to or to about 5%, from or from about 1% to or to about 4%, from or from about 1% to or to about 3%, from or from about 1% to or to about 2%, from or from about 2% to or to about 10%, from or from about 2% to or to about 9%, from or from about 2% to or to about 8%, from or from about 2% to or to about 7%, from or from about 2% to or to about 6%, from or from about 2% to or to about 5%, from or from about 2% to or to about 4%, from or from about 2% to or to about 3%, from or from about 3% to or to about 10%, from or from about 3% to or to about 9%, from or from about 3% to or to about 8%, from or from about 3% to or to about 7%, from or from about 3% to or to about 6%, from or from about 3% to or to about 5%, from or from about 3% to or to about 4%, from or from about 4% to or to about 10%, from or from about 4% to or to about 9%, from or from about 4% to or to about 8%, from or from about 4% to or to about 7%, from or from about 4% to or to about 6%, from or from about 4% to or to about 5%, from or from about 5% to or to about 10%, from or from about 5% to or to about 9%, from or from about 4% to or to about 8%, from or from about 5% to or to about 7%, from or from about 5% to or to about 6%, from or from about 6% to or to about 10%, from or from about 6% to or to about 9%, from or from about 6% to or to about 8%, from or from about 6% to or to about 7%, from or from about 7% to or to about 10%, from or from about 7% to or to about 9%, from or from about 7% to or to about 8%, from or from about 8% to or to about 10%, from or from about 8% to or to about 9%, or from or from about 9% to or to about 10% v/v plasma, e.g., human plasma. In some embodiments, the culture medium comprises and/or is supplemented with from 0.8% to 1.2% v/v human plasma. In some embodiments, the culture medium comprises and/or is supplemented with 1.0% v/v human plasma. In some embodiments, the culture medium comprises and/or is supplemented with about 1.0% v/v human plasma.

In some embodiments, the culture medium comprises serum, e.g., human serum. In some embodiments, the culture medium is supplemented with serum, e.g., human serum. In some embodiments, the serum is inactivated, e.g., heat inactivated. In some embodiments, the serum is filtered, e.g., sterile-filtered.

In some embodiments, the culture medium comprises glutamine. In some embodiments, the culture medium is supplemented with glutamine. In some embodiments, the culture medium comprises and/or is supplemented with from or from about 2.0 to or to about 6.0 mM glutamine. In some embodiments, the culture medium comprises and/or is supplemented with from or from about 2.0 to or to about 5.5, from or from about 2.0 to or to about 5.0, from or from about 2.0 to or to about 4.5, from or from about 2.0 to or to about 4.0, from or from about 2.0 to or to about 3.5, from or from about 2.0 to or to about 3.0, from or from about 2.0 to or to about 2.5, from or from about 2.5 to or to about 6.0, from or from about 2.5 to or to about 5.5, from or from about 2.5 to or to about 5.0, from or from about 2.5 to or to about 4.5, from or from about 2.5 to or to about 4.0, from or from about 2.5 to or to about 3.5, from or from about 2.5 to or to about 3.0, from or from about 3.0 to or to about 6.0, from or from about 3.0 to or to about 5.5, from or from about 3.0 to or to about 5.0, from or from about 3.0 to or to about 4.5, from or from about 3.0 to or to about 4.0, from or from about 3.0 to or to about 3.5, from or from about 3.5 to or to about 6.0, from or from about 3.5 to or to about 5.5, from or from about 3.5 to or to about 5.0, from or from about 3.5 to or to about 4.5, from or from about 3.5 to or to about 4.0, from or from about 4.0 to or to about 6.0, from or from about 4.0 to or to about 5.5, from or from about 4.0 to or to about 5.0, from or from about 4.0 to or to about 4.5, from or from about 4.5 to or to about 6.0, from or from about 4.5 to or to about 5.5, from or from about 4.5 to or to about 5.0, from or from about 5.0 to or to about 6.0, from or from about 5.0 to or to about 5.5, or from or from about 5.5 to or to about 6.0 mM glutamine. In some embodiments, the culture medium comprises and/or is supplemented with from 3.2 mM glutamine to 4.8 mM glutamine. In some embodiments, the culture medium comprises and/or is supplemented with 4.0 mM glutamine. In some embodiments, the culture medium comprises and/or is supplemented with about 4.0 mM glutamine.

In some embodiments, the culture medium comprises one or more cyotkines. In some embodiments, the culture medium is supplemented with one or more cyotkines.

In some embodiments, the cytokine is selected from IL-2, IL-12, IL-15, IL-18, and combinations thereof.

In some embodiments, the culture medium comprises and/or is supplemented with IL-2. In some embodiments, the culture medium comprises and/or is supplemented with from or from about 150 to or to about 2,500 IU/mL IL-2. In some embodiments, the culture medium comprises and/or is supplemented with from or from about 200 to or to about 2,250, from or from about 200 to or to about 2,000, from or from about 200 to or to about 1,750, from or from about 200 to or to about 1,500, from or from about 200 to or to about 1,250, from or from 200 to or to about 1,000, from or from about 200 to or to about 750, from or from about 200 to or to about 500, from or from about 200 to or to about 250, from or from about 250 to or to about 2,500, from or from about 250 to or to about 2,250, from or from about 250 to or to about 2,000, from or from about 250 to or to about 1,750, from or from about 250 to or to about 1,500, from or from about 250 to or to about 1,250, from or from about 250 to or to about 1,000, from or from about 250 to or to about 750, from or from about 250 to or to about 500, from or from about 500 to or to about 2,500, from or from about 500 to or to about 2,250, from or from about 500 to or to about 2,000, from or from about 500 to or to about 1,750, from or from about 500 to or to about 1,500, from or from about 500 to or to about 1,250, from or from about 500 to or to about 1,000, from or from about 500 to or to about 750, from or from about 750 to or to about 2,250, from or from about 750 to or to about 2,000, from or from about 750 to or to about 1,750, from or from about 750 to or to about 1,500, from or from about 750 to or to about 1,250, from or from about 750 to or to about 1,000, from or from about 1,000 to or to about 2,500, from or from about 1,000 to or to about 2,250, from or from about 1,000 to or to about 2,000, from or from about 1,000 to or to about 1,750, from or from about 1,000 to or to about 1,500, from or from about 1,000 to or to about 1,250, from or from about 1,250 to or to about 2,500, from or from about 1,250 to or to about 2,250, from or from about 1,250 to or to about 2,000, from or from about 1,250 to or to about 1,750, from or from about 1,250 to or to about 1,500, from or from about 1,500 to or to about 2,500, from or from about 1,500 to or to about 2,250, from or from about 1,500 to or to about 2,000, from or from about 1,500 to or to about 1,750, from or from about 1,750 to or to about 2,500, from or from about 1,750 to or to about 2,250, from or from about 1,750 to or to about 2,000, from or from about 2,000 to or to about 2,500, from or from about 2,000 to or to about 2,250, or from or from about 2,250 to or to about 2,500 IU/mL IL-2.

In some embodiments, the culture medium comprises and/or is supplemented with from 64 μg/L to 96 μg/L IL-2. In some embodiments, the culture medium comprises and/or is supplemented with 80 μg/L IL-2 (approximately 1,333 IU/mL). In some embodiments, the culture medium comprises and/or is supplemented with about 80 μg/L.

In some embodiments, the culture medium comprises and/or is supplemented with a combination of IL-2 and IL-15.

In some embodiments, the culture medium comprises and/or is supplemented with a combination of IL-2, IL-15, and IL-18.

In some embodiments, the culture medium comprises and/or is supplemented with a combination of IL-2, IL-18, and IL-21.

In some embodiments, the culture medium comprises and/or is supplemented with glucose. In some embodiments, the culture medium comprises and/or is supplemented with from or from about 0.5 to or to about 3.5 g/L glucose. In some embodiments, the culture medium comprises and/or is supplemented with from or from about 0.5 to or to about 3.0, from or from about 0.5 to or to about 2.5, from or from about 0.5 to or to about 2.0, from or from about 0.5 to or to about 1.5, from or from about 0.5 to or to about 1.0, from or from about 1.0 to or to about 3.0, from or from about 1.0 to or to about 2.5, from or from about 1.0 to or to about 2.0, from or from about 1.0 to or to about 1.5, from or from about 1.5 to or to about 3.0, from or from about 1.5 to or to about 2.5, from or from about 1.5 to or to about 2.0, from or from about 2.0 to or to about 3.0, from or from about 2.0 to or to about 2.5, or from or from about 2.5 to or to about 3.0 g/L glucose. In some embodiments, the culture medium comprises and/or is supplemented with from 1.6 to 2.4 g/L glucose. In some embodiments, the culture medium comprises and/or is supplemented with 2.0 g/L glucose. In some embodiments, the culture medium comprises about 2.0 g/L glucose.

In some embodiments, the culture medium comprises and/or is supplemented with sodium pyruvate. In some embodiments, the culture medium comprises and/or is supplemented with from or from about 0.1 to or to about 2.0 mM sodium pyruvate. In some embodiments, the culture medium comprises and/or is supplemented with from or from about 0.1 to or to about 1.8, from or from about 0.1 to or to about 1.6, from or from about 0.1 to or to about 1.4, from or from about 0.1 to or to about 1.2, from or from about 0.1 to or to about 1.0, from or from about 0.1 to or to about 0.8, from or from about 0.1 to or to about 0.6, from or from about 0.1 to or to about 0.4, from or from about 0.1 to or to about 0.2, from or from about 0.2 to or to about 2.0, from or from about 0.2 to or to about 1.8, from or from about 0.2 to or to about 1.6, from or from about 0.2 to or to about 1.4, from or from about 0.2 to or to about 1.2, from or from about 0.2 to or to about 1.0, from or from about 0.2 to or to about 0.8, from or from about 0.2 to or to about 0.6, from or from about 0.2 to or to about 0.4, from or from about 0.4 to or to about 2.0, from or from about 0.4 to or to about 1.8, from or from about 0.4 to or to about 1.6, from or from about 0.4 to or to about 1.4, from or from about 0.4 to or to about 1.2, from or from about 0.4 to or to about 1.0, from or from about 0.4 to or to about 0.8, from or from about 0.4 to or to about 0.6, from or from about 0.6 to or to about 2.0, from or from about 0.6 to or to about 1.8, from or from about 0.6 to or to about 1.6, from or from about 0.6 to or to about 1.4, from or from about 0.6 to or to about 1.2, from or from about 0.6 to or to about 1.0, from or form about 0.6 to or to about 0.8, from or from about 0.8 to or to about 2.0, from or from about 0.8 to or to about 1.8, from or from about 0.8 to or to about 1.6, from or from about 0.8 to or to about 1.4, from or from about 0.8 to or to about 1.4, from or from about 0.8 to or to about 1.2, from or from about 0.8 to or to about 1.0, from or from about 1.0 to or to about 2.0, from or from about 1.0 to or to about 1.8, from or from about 1.0 to or to about 1.6, from or from about 1.0 to or to about 1.4, from or from about 1.0 to or to about 1.2, from or from about 1.2 to or to about 2.0, from or from about 1.2 to or to about 1.8, from or from about 1.2 to or to about 1.6, from or from about 1.2 to or to about 1.4, from or from about 1.4 to or to about 2.0, from or from about 1.4 to or to about 1.8, from or from about 1.4 to or to about 1.6, from or from about 1.6 to or to about 2.0, from or from about 1.6 to or to about 1.8, or from or from about 1.8 to or to about 2.0 mM sodium pyruvate. In some embodiments, the culture medium comprises from 0.8 to 1.2 mM sodium pyruvate. In some embodiments, the culture medium comprises 1.0 mM sodium pyruvate. In some embodiments, the culture medium comprises about 1.0 mM sodium pyuruvate.

In some embodiments, the culture medium comprises and/or is supplemented with sodium hydrogen carbonate. In some embodiments, the culture medium comprises and/or is supplemented with from or from about 0.5 to or to about 3.5 g/L sodium hydrogen carbonate. In some embodiments, the culture medium comprises and/or is supplemented with from or from about 0.5 to or to about 3.0, from or from about 0.5 to or to about 2.5, from or from about 0.5 to or to about 2.0, from or from about 0.5 to or to about 1.5, from or from about 0.5 to or to about 1.0, from or from about 1.0 to or to about 3.0, from or from about 1.0 to or to about 2.5, from or from about 1.0 to or to about 2.0, from or from about 1.0 to or to about 1.5, from or from about 1.5 to or to about 3.0, from or from about 1.5 to or to about 2.5, from or from about 1.5 to or to about 2.0, from or from about 2.0 to or to about 3.0, from or from about 2.0 to or to about 2.5, or from or from about 2.5 to or to about 3.0 g/L sodium hydrogen carbonate. In some embodiments, the culture medium comprises and/or is supplemented with from 1.6 to 2.4 g/L sodium hydrogen carbonate. In some embodiments, the culture medium comprises and/or is supplemented with 2.0 g/L sodium hydrogen carbonate. In some embodiments, the culture medium comprises about 2.0 g/L sodium hydrogen carbonate.

In some embodiments, the culture medium comprises and/or is supplemented with albumin, e.g., human albumin, e.g., a human albumin solution described herein. In some embodiments, the culture medium comprises and/or is supplemented with from or from about 0.5% to or to about 3.5% v/v of a 20% albumin solution, e.g., a 20% human albumin solution. In some embodiments, the culture medium comprises and/or is supplemented with from or from about 0.5% to or to about 3.0%, from or from about 0.5% to or to about 2.5%, from or from about 0.5% to or to about 2.0%, from or from about 0.5% to or to about 1.5%, from or from about 0.5% to or to about 1.0%, from or from about 1.0% to or to about 3.0%, from or from about 1.0% to or to about 2.5%, from or from about 1.0% to or to about 2.0%, from or from about 1.0% to or to about 1.5%, from or from about 1.5% to or to about 3.0%, from or from about 1.5% to or to about 2.5%, from or from about 1.5% to or to about 2.0%, from or from about 2.0% to or to about 3.0%, from or from about 2.0% to or to about 2.5%, or from or from about 2.5% to or to about 3.0% v/v of a 20% albumin solution, e.g., a 20% human albumin solution. In some embodiments, the culture medium comprises and/or is supplemented with from 1.6% to 2.4% v/v of a 20% albumin solution, e.g., a 20% human albumin solution. In some embodiments, the culture medium comprises and/or is supplemented with 2.0% v/v of a 20% albumin solution, e.g., a 20% human albumin solution. In some embodiments, the culture medium comprises about 2.0% v/v of a 20% albumin solution, e.g., a 20% human albumin solution.

In some embodiments, the culture medium comprises and/or is supplemented with from or from about 2 to or to about 6 g/L albumin, e.g., human albumin. In some embodiments, the culture medium comprises and/or is supplemented with from or from about 2 to or to about 5.5, from or from about 2 to or to about 5.0, from or from about 2 to or to about 4.5, from or from about 2 to or to about 4, from or from about 2 to or to about 3.5, from or from about 2 to or to about 3, from or from about 2 to or to about 2.5, from or from about 2.5 to or to about 6, from or from about 2.5 to or to about 5.5, from or from about 2.5 to or to about 5.5, from or from about 2.5 to or to about 5.0, from or from about 2.5 to or to about 4.5, from or from about 2.5 to or to about 4.0, from or from about 2.5 to or to about 3.5, from or from about 2.5 to or to about 3.0, from or from about 3 to or to about 6, from or from about 3 to or to about 5.5, from or from about 3 to or to about 5, from or from about 3 to or to about 4.5, from or from about 3 to or to about 4, from or from about 3 to or to about 3.5, from or from about 3.5 to or to about 6, from or from about 3.5 to or to about 5.5, from or from about 3.5 to or to about 5, from or from about 3.5 to or to about 4.5, from or from about 3.5 to or to about 4, from or from about 4 to or to about 6, from or from about 4 to or to about 5.5, from or from about 4 to or to about 5, from or from about 4 to or to about 4.5, from or from about 4.5 to or to about 6, from or from about 4.5 to or to about 5.5, from or from about 4.5 to or to about 5, from or from about 5 to or to about 6, from or from about 5 to or to about 5.5, or from or from about 5.5 to or to about 6 g/L albumin, e.g., human albumin. In some embodiments, the culture medium comprises and/or is supplemented with from 3.2 to 4.8 g/L albumin, e.g., human albumin. In some embodiments, the culture medium comprises 4 g/L albumin, e.g., human albumin. In some embodiments, the culture medium comprises about 4 g/L albumin, e.g., human albumin

In some embodiments, the culture medium is supplemented with Poloxamer 188. In some embodiments, the culture medium comprises and/or is supplemented with from or from about 0.1 to or to about 2.0 g/L Poloxamer 188. In some embodiments, the culture medium comprises and/or is supplemented with from or from about 0.1 to or to about 1.8, from or from about 0.1 to or to about 1.6, from or from about 0.1 to or to about 1.4, from or from about 0.1 to or to about 1.2, from or from about 0.1 to or to about 1.0, from or from about 0.1 to or to about 0.8, from or from about 0.1 to or to about 0.6, from or from about 0.1 to or to about 0.4, from or from about 0.1 to or to about 0.2, from or from about 0.2 to or to about 2.0, from or from about 0.2 to or to about 1.8, from or from about 0.2 to or to about 1.6, from or from about 0.2 to or to about 1.4, from or from about 0.2 to or to about 1.2, from or from about 0.2 to or to about 1.0, from or from about 0.2 to or to about 0.8, from or from about 0.2 to or to about 0.6, from or from about 0.2 to or to about 0.4, from or from about 0.4 to or to about 2.0, from or from about 0.4 to or to about 1.8, from or from about 0.4 to or to about 1.6, from or from about 0.4 to or to about 1.4, from or from about 0.4 to or to about 1.2, from or from about 0.4 to or to about 1.0, from or from about 0.4 to or to about 0.8, from or from about 0.4 to or to about 0.6, from or from about 0.6 to or to about 2.0, from or from about 0.6 to or to about 1.8, from or from about 0.6 to or to about 1.6, from or from about 0.6 to or to about 1.4, from or from about 0.6 to or to about 1.2, from or from about 0.6 to or to about 1.0, from or form about 0.6 to or to about 0.8, from or from about 0.8 to or to about 2.0, from or from about 0.8 to or to about 1.8, from or from about 0.8 to or to about 1.6, from or from about 0.8 to or to about 1.4, from or from about 0.8 to or to about 1.4, from or from about 0.8 to or to about 1.2, from or from about 0.8 to or to about 1.0, from or from about 1.0 to or to about 2.0, from or from about 1.0 to or to about 1.8, from or from about 1.0 to or to about 1.6, from or from about 1.0 to or to about 1.4, from or from about 1.0 to or to about 1.2, from or from about 1.2 to or to about 2.0, from or from about 1.2 to or to about 1.8, from or from about 1.2 to or to about 1.6, from or from about 1.2 to or to about 1.4, from or from about 1.4 to or to about 2.0, from or from about 1.4 to or to about 1.8, from or from about 1.4 to or to about 1.6, from or from about 1.6 to or to about 2.0, from or from about 1.6 to or to about 1.8, or from or from about 1.8 to or to about 2.0 g/L Poloxamer 188. In some embodiments, the culture medium comprises from 0.8 to 1.2 g/L Poloxamer 188. In some embodiments, the culture medium comprises 1.0 g/L Poloxamer 188. In some embodiments, the culture medium comprises about 1.0 g/L Poloxamer 188.

In some embodiments, the culture medium comprises and/or is supplemented with one or more antibiotics.

A first exemplary culture medium is set forth in Table 1.

TABLE 1
Exemplary Culture Medium #1

| | Exemplary | | Exemplary | |
|---|---|---|---|---|
| Component | Concentration Range | | Concentration | |
| CellgroSCGM liquid medium | undiluted | | undiluted | |
| Human Plasma | 0.8-1.2% | (v/v) | 1.0% | v/v |
| Glutamine | 3.2-4.8 | mM | 4.0 | mM |
| IL-2 | 64-96 | μg/L | 80 | μg/L |

A second exemplary culture medium is set forth in Table 2.

TABLE 2
Exemplary Culture Medium #2

| | Exemplary | | Exemplary | |
|---|---|---|---|---|
| Component | Concentration Range | | Concentration | |
| RPMI1640 | 7.6-13.2 | g/L | 10.4 | g/L |
| Human Plasma | 0.8-1.2% | (v/v) | 1.0% | v/v |
| Glucose | 1.6-2.4 | g/L | 2.0 | g/L |
| Glutamine | 3.2-4.8 | mM | 4.0 | mM |
| Sodium Pyruvate | 0.8-1.2 | mM | 1.0 | mM |
| Sodium Hydrogen Carbonate | 1.6-2.4 | g/L | 2.0 | g/L |
| IL-2 | 64-96 | μg/L | 80 | μg/L |
| Albumin 20% solution | 1.6-2.5% v/v | | 2.0% v/v | |
| | (3.2 to 4.8 g/L) | | (4.0 g/L) | |
| Poloxamer 188 | 0.8-1.2 | g/L | 1.0 | g/L |

### 2. CD3 Binding Antibodies

In some embodiments, the culture medium comprises and/or is supplemented with a CD3 binding antibody or antigen binding fragment thereof. In some embodiments, the CD3 binding antibody or antigen binding fragment thereof is selected from the group consisting of OKT3, UCHT1, and HIT3a, or variants thereof. In some embodiments, the CD3 binding antibody or antigen binding fragment thereof is OKT3 or an antigen binding fragment thereof.

In some embodiments, the CD3 binding antibody or antigen binding fragment thereof and feeder cells are added to the culture vessel before addition of NK cells and/or culture medium.

In some embodiments, the culture medium comprises and/or is supplemented with from or from about 5 ng/ml to or to about 15 ng/mL OKT3. In some embodiments, the culture medium comprises and/or is supplemented with from or from about 5 to or to about 12.5, from or from about 5 to or to about 10, from or from about 5 to or to about 7.5, from or from about 7.5 to or to about 15, from or from about 7.5 to or to about 12.5, from or from about 7.5 to or to about 10, from or from about 10 to or to about 15, from or from about 10 to or to about 12.5, or from or from about 12.5 to or to about 15 ng/mL OKT3. In some embodiments, the culture medium comprises and/or is supplemented with 10 ng/mL OKT3. In some embodiments, the culture medium comprises and/or is supplemented with about 10 ng/ml OKT3.

### 3. Culture Vessels

A number of vessels are consistent with the disclosure herein. In some embodiments, the culture vessel is selected from the group consisting of a flask, a bottle, a dish, a multiwall plate, a roller bottle, a bag, and a bioreactor.

In some embodiments, the culture vessel is treated to render it hydrophilic. In some embodiments, the culture vessel is treated to promote attachment and/or proliferation. In some embodiments, the culture vessel surface is coated with serum, collagen, laminin, gelatin, poy-L-lysine, fibronectin, extracellular matrix proteins, and combinations thereof.

In some embodiments, different types of culture vessels are used for different stages of culturing.

In some embodiments, the culture vessel has a volume of from or from about 100 mL to or to about 1,000 L. In some embodiments, the culture vessel has a volume of or about 125 mL, of or about 250 mL, of or about 500 mL, of or about 1 L, of or about 5 L, of about 10 L, or of or about 20 L.

In some embodiments, the culture vessel is a bioreactor.

In some embodiments, the bioreactor is a rocking bed (wave motion) bioreactor. In some embodiments, the bioreactor is a stirred tank bioreactor. In some embodiments, the bioreactor is a rotating wall vessel. In some embodiments, the bioreactor is a perfusion bioreactor. In some embodiments, the bioreactor is an isolation/expansion automated system. In some embodiments, the bioreactor is an automated or semi-automated bioreactor. In some embodiments, the bioreactor is a disposable bag bioreactor.

In some embodiments, the bioreactor has a volume of from about 100 mL to about 1,000 L. In some embodiments, the bioreactor has a volume of from about 10 L to about 1,000 L. In some embodiments, the bioreactor has a volume of from about 100 L to about 900 L. In some embodiments, the bioreactor has a volume of from about 10 L to about 800 L. In some embodiments, the bioreactor has a volume of from about 10 L to about 700 L, about 10 L to about 600 L, about 10 L to about 500 L, about 10 L to about 400 L, about 10 L to about 300 L, about 10 L to about 200 L, about 10 L to about 100 L, about 10 L to about 90 L, about 10 L to about 80 L, about 10 L to about 70 L, about 10 L to about 60 L, about 10 L to about 50 L, about 10 L to about 40 L, about 10 L to about 30 L, about 10 L to about 20 L, about 20 L to about 1,000 L, about 20 L to about 900 L, about 20 L to about 800 L, about 20 L to about 700 L, about 20 L to about 600 L, about 20 L to about 500 L, about 20 L to about 400 L, about 20 L to about 300 L, about 20 L to about 200 L, about 20 L to about 100 L, about 20 L to about 90 L, about 20 L to about 80 L, about 20 L to about 70 L, about 20 L to about 60 L, about 20 L to about 50 L, about 20 L to about 40 L, about 20 L to about 30 L, about 30 L to about 1,000 L, about 30 L to about 900 L, about 30 L to about 800 L, about 30 L to about 700 L, about 30 L to about 600 L, about 30 L to about 500 L, about 30 L to about 400 L, about 30 L to about 300 L, about 30 L to about 200 L, about 30 L to about 100 L, about 30 L to about 90 L, about 30 L to about 80 L, about 30 L to about 70 L, about 30 L to about 60 L, about 30 L to about 50 L, about 30 L to about 40 L, about 40 L to about 1,000 L, about 40 L to about 900 L, about 40 L to about 800 L, about 40 L to about 700 L, about 40 L to about 600 L, about 40 L to about 500 L, about 40 L to about 400 L, about 40 L to about 300 L, about 40 L to about 200 L, about 40 L to about 100 L, about 40 L to about 90 L, about 40 L to about 80 L, about 40 L to about 70 L, about 40 L to about 60 L, about 40 L to about 50 L, about 50 L to about 1,000 L, about 50 L to about 900 L, about 50 L to about 800 L, about 50 L to about 700 L, about 50 L to about 600 L, about 50 L to about 500 L, about 50 L to about 400 L, about 50 L to about 300 L, about 50 L to about 200 L, about 50 L to about 100 L, about 50 L to about 90 L, about 50 L to about 80 L, about 50 L to about 70 L, about 50 L to about 60 L, about 60 L to about 1,000 L, about 60 L to about 900 L, about 60 L to about 800 L, about 60 L to about 700 L, about 60 L to about 600 L, about 60 L to about 500 L, about 60 L to about 400 L, about 60 L to about 300 L, about 60 L to about 200 L, about 60 L to about 100L, about 60 L to about 90 L, about 60 L to about 80 L, about 60 L to about 70 L, about 70 L to about 1,000 L, about 70 L to about 900 L, about 70 L to about 800 L, about 70 L to about 700 L, about 70 L to about 600 L, about 70 L to about 500 L, about 70 L to about 400 L, about 70 L to about 300 L, about 70 L to about 200 L, about 70 L to about 100 L, about 70 L to about 90 L, about 70 L to about 80 L, about 80 L to about 1,000 L, about 80 L to about 900 L, about 80 L to about 800 L, about 80 L to about 700 L, about 80 L to about 600 L, about 80 L to about 500 L, about 80 L to about 400 L, about 80 L to about 300 L, about 80 L to about 200 L, about 80 L to about 100 L, about 80 L to about 90 L, about 90 L to about 1,000 L, about 90 L to about 900 L, about 90 L to about 800 L, about 90 L to about 700 L, about 90 L to about 600 L, about 90 L to about 500 L, about 90 L to about 400 L, about 90 L to about 300 L, about 90 L to about 200 L, about 90 L to about 100 L, about 100 L to about 1,000 L, about 100 L to about 900 L, about 100 L to about 800 L, about 100 L to about 700 L, about 100 L toa bout 600 L, about 100 L to about 500 L, about 100 L to about 400 L, about 100 L to about 300 L, about 100 L to about 200 L, about 200 L to about 1,000 L, about 200 L to about 900 L, about 200 L to about 800 L, about 200 L to about 700 L, about 200 L to about 600 L, about 200 L to about 500 L, about 200 L to about 400 L, about 200 L to about 300 L, about 300 L to about 1,000 L, about 300 L to about 900 L, about 300 L to about 800 L, about 300 L to about 700 L, about 300 L to about 600 L, about 300 L to about 500 L, about 300 L to about 400 L, about 400 L to about 1,000 L, about 400 L to about 900 L, about 400 L to about 800 L, about 400 L to about 700 L, about 400 L to about 600 L, about 400 L to about 500 L, about 500 L to about 1,000 L, about 500 L to about 900 L, about 500 L to about 800 L, about 500 L to about 700 L, about 500 L to about 600 L, about 600 L to about 1,000 L, about 600 L to about 900 L, about 600 L to about 800 L, about 600 L to about 700 L, about 700 L to about 1,000 L, about 700 L to about 900 L, about 700 L to about 800 L, about 800 L to about 1,000 L, about 800 L to about 900 L, or about 900 L to about 1,000 L. In some embodiments, the bioreactor has a volume of about 50 L.

In some embodiments, the bioreactor has a volume of from 100 mL to 1,000 L. In some embodiments, the bioreactor has a volume of from 10 L to 1,000 L. In some embodiments, the bioreactor has a volume of from 100 L to 900 L. In some embodiments, the bioreactor has a volume of from 10 L to 800 L. In some embodiments, the bioreactor has a volume of from 10 L to 700 L, 10 L to 600 L, 10 L to 500 L, 10 L to 400 L, 10 L to 300 L, 10 L to 200 L, 10 L to 100 L, 10 L to 90 L, 10 L to 80 L, 10 L to 70 L, 10 L to 60 L, 10 L to 50 L, 10 L to 40 L, 10 L to 30 L, 10 L to 20 L, 20 L to 1,000 L, 20 L to 900 L, 20 L to 800 L, 20 L to 700 L, 20 L to 600 L, 20 L to 500 L, 20 L to 400 L, 20 L to 300 L, 20 L to 200 L, 20 L to 100 L, 20 L to 90 L, 20 L to 80 L, 20 L to 70 L, 20 L to 60 L, 20 L to 50 L, 20 L to 40 L, 20 L to 30 L, 30 L to 1,000 L, 30 L to 900 L, 30 L to 800 L, 30 L to 700 L, 30 L to 600 L, 30 L to 500 L, 30 L to 400 L, 30 L to 300 L, 30 L to 200 L, 30 L to 100 L, 30 L to 90 L, 30 L to 80 L, 30 L to 70 L, 30 L to 60 L, 30 L to 50 L, 30 L to 40 L, 40 L to 1,000 L, 40 L to 900 L, 40 L to 800 L, 40 L to 700 L, 40 L to 600 L, 40 L to 500 L, 40 L to 400 L, 40 L to 300 L, 40 L to 200 L, 40 L to 100 L, 40 L to 90 L, 40 L to 80 L, 40 L to 70 L, 40 L to 60 L, 40 L to 50 L, 50 L to 1,000 L, 50 L to 900 L, 50 L to 800 L, 50 L to 700 L, 50 L to 600 L, 50 L to 500 L, 50 L to 400 L, 50 L to 300 L, 50 L to 200 L, 50 L to 100 L, 50 L to 90 L, 50 L to 80 L, 50 L to 70 L, 50 L to 60 L, 60 L to 1,000 L, 60 L to 900 L, 60 L to 800 L, 60 L to 700 L, 60 L to 600 L, 60 L to 500 L, 60 L to 400 L, 60 L to 300 L, 60 L to 200 L, 60 L to 100L, 60 L to 90 L, 60 L to 80 L, 60 L to 70 L, 70 L to 1,000 L, 70 L to 900 L, 70 L to 800 L, 70 L to 700 L, 70 L to 600 L, 70 L to 500 L, 70 L to 400 L, 70 L to 300 L, 70 L to 200 L, 70 L to 100 L, 70 L to 90 L, 70 L to 80 L, 80 L to 1,000 L, 80 L to 900 L, 80 L to 800 L, 80 L to 700 L, 80 L to 600 L, 80 L to 500 L, 80 L to 400 L, 80 L to 300 L, 80 L to 200 L, 80 L to 100 L, 80 L to 90 L, 90 L to 1,000 L, 90 L to 900 L, 90 L to 800 L, 90 L to 700 L, 90 L to 600 L, 90 L to 500 L, 90 L to 400 L, 90 L to 300 L, 90 L to 200 L, 90 L to 100 L, 100 L to 1,000 L, 100 L to 900 L, 100 L to 800 L, 100 L to 700 L, 100 L to 600 L, 100 L to 500 L, 100 L to 400 L, 100 L to 300 L, 100 L to 200 L, 200 L to 1,000 L, 200 L to 900 L, 200 L to 800 L, 200 L to 700 L, 200 L to 600 L, 200 L to 500 L, 200 L to 400 L, 200 L to 300 L, 300 L to 1,000 L, 300 L to 900 L, 300 L to 800 L, 300 L to 700 L, 300 L to 600 L, 300 L to 500 L, 300 L to 400 L, 400 L to 1,000 L, 400 L to 900 L, 400 L to 800 L, 400 L to 700 L, 400 L to 600 L, 400 L to 500 L, 500 L to 1,000 L, 500 L to 900 L, 500 L to 800 L, 500 L to 700 L, 500 L to 600 L, 600 L to 1,000 L, 600 L to 900 L, 600 L to 800 L, 600 L to 700 L, 700 L to 1,000 L, 700 L to 900 L, 700 L to 800 L, 800 L to 1,000 L, 800 L to 900 L, or 900 L to 1,000 L. In some embodiments, the bioreactor has a volume of 50 L.

### 4. Cell Expansion and Stimulation

In some embodiments, the natural killer cell source, e.g., single unit of cord blood, is co-cultured with feeder cells to produce expanded and stimulated NK cells.

In some embodiments, the co-culture is carried out in a culture medium described herein, e.g., exemplary culture medium #1 (Table 1) or exemplary culture medium #2 (Table 2).

In some embodiments, the natural killer cell source, e.g., single unit of cord blood, comprises from or from about 1×10⁷ to or to about 1×10⁹ total nucleated cells prior to expansion. In some embodiments, the natural killer cell source, e.g., single unit of cord blood, comprises from or from about 1×10⁸ to or to about 1.5×10⁸ total nucleated cells prior to expansion. In some embodiments, the natural killer cell source, e.g., single unit of cord blood, comprises 1×10⁸ total nucleated cells prior to expansion. In some embodiments, the natural killer cell source, e.g., single unit of cord blood, comprises about 1×10⁸ total nucleated cells prior to expansion. In some embodiments, the natural killer cell source, e.g., single unit of cord blood, comprises 1×10⁹ total nucleated cells prior to expansion. In some embodiments, the natural killer cell source, e.g., single unit of cord blood, comprises about 1×10⁹ total nucleated cells prior to expansion.

In some embodiments, cells from the co-culture of the natural killer cell source, e.g., single unit of cord blood and feeder cells are harvested and frozen, e.g., in a cryopreservation composition described herein. In some embodiments, the frozen cells from the co-culture are an infusion-ready drug product. In some embodiments, the frozen cells from the co-culture are used as a master cell bank (MCB) from which to produce an infusion-ready drug product, e.g., through one or more additional co-culturing steps, as described herein. Thus, for example, a natural killer cell source can be expanded and stimulated as described herein to produce expanded and stimulated NK cells suitable for use in an infusion-ready drug product without generating any intermediate products. A natural killer cell source can also be expanded and stimulated as described herein to produce an intermediate product, e.g., a first master cell bank (MCB). The first MCB can be used to produce expanded and stimulated NK cells suitable for use in an infusion-ready drug product, or, alternatively, be used to produce another intermediate product, e.g., a second MCB. The second MCB can be used to produce expanded and stimulated NK cells suitable for an infusion-ready drug product, or alternatively, be used to produce another intermediate product, e.g., a third MCB, and so on.

In some embodiments, the ratio of feeder cells to cells of the natural killer cell source or MCB cells inoculated into the co-culture is from or from about 1:1 to or to about 4:1. In some embodiments, the ratio of feeder cells to cells of the natural killer cell source or MCB cells is from or from about 1:1 to or to about 3.5:1, from or from about 1:1 to or to about 3:1, from or from about 1:1 to or to about 2.5:1, from or from about 1.1 to or to about 2:1, from or from about 1:1 to or to about 1.5:1, from or from about 1.5:1 to or to about 4:1, from or from about 1.5:1 to or to about 3.5:1, from or from about 1.5:1 to or to about 3:1, from or from about 1.5:1 to or to about 2.5:1, from or from about 1.5:1 to or to about 2:1, from or from about 2:1 to or to about 4:1, from or from about 2:1 to or to about 3.5:1, from or from about 2:1 to or to about 3:1, from or from about 2:1 to or to about 2.5:1, from or from about 2.5:1 to or to about 4:1, from or from about 2.5:1 to or to about 3.5:1, from or from about 2.5:1 to or to about 3:1, from or from about 3:1 to or to about 4:1, from or from about 3:1 to or to about 3.5:1, or from or from about 3.5:1 to or to about 4:1. In some embodiments, the ratio of feeder cells to cells of the natural killer cell source or MCB inoculated into the co-culture is 2.5:1. In some embodiments, the ratio of feeder cells to cells of the natural killer cell source or MCB inoculated into the co-culture is about 2.5:1.

In some embodiments, the co-culture is carried out in a disposable culture bag, e.g., a 1L disposable culture bag. In some embodiments, the co-culture is carried out in a bioreactor, e.g., a 50L bioreactor. In some embodiments, culture medium is added to the co-culture after the initial inoculation.

In some embodiments, the co-culture is carried out for 1, 2, 3, 4, 5, 6, 7, 8, 9, 10, 11, 12, 13, 14, 15, 16, 17, 18, 19, 20, 21, 22, 23, 24 or more days. In some embodiments, the co-culture is carried out for a maximum of 16 days.

In some embodiments, the co-culture is carried out at 37° C. or about 37° C.

In some embodiments, the co-culture is carried out at pH 7.9 or about pH 7.9.

In some embodiments, the co-culture is carried out at a dissolved oxygen (DO) level of 50% or more.

In some embodiments, exemplary culture medium #1 (Table 1) is used to produce a MCB and exemplary culture medium #2 (Table 2) is used to produce cells suitable for an infusion-ready drug product.

In some embodiments, the co-culture of the natural killer cell source, e.g., single unit of cord blood, with feeder cells yields from or from about 50×10⁸ to or to about 50×10¹² cells, e.g., MCB cells or infusion-ready drug product cells. In some embodiments, the expansion yields from or from about 50×10⁸ to or to about 25×10¹⁰, from or from about 10×10⁸ to or to about 1×10¹⁰, from or from about 50×10⁸ to or to about 75×10⁹, from or from about 50×10⁸ to or to about 50×10⁹, from or from about 50×10⁸ to or to about 25×10⁹, from or from about 50×10⁸ to or to about 1×10⁹, from or from about 50×10⁸ to or to about 75×10⁸, from or from about 75×10⁸ to or to about 50×10¹⁰, from or from about 75×10⁸ to or to about 25×10¹⁰, from or from about 75×10⁸ to or to about 1×10¹⁰, from or from about 75×10⁸ to or to about 75×10⁹, from or from about 75×10⁸ to or to about 50×10⁹, from or from about 75×10⁸ to or to about 25×10⁹, from or from about 75×10⁸ to or to about 1×10⁹, from or from about 1×10⁹ to or to about 50×10¹⁰, from or from about 1×10⁹ to or to about 25×10¹⁰, from or from about 1×10⁹ to or to about 1×10¹⁰, from or from about 1×10⁹ to or to about 75×10⁹, from or from about 1×10⁹ to or to about 50×10⁹, from or from about 1×10⁹ to or to about 25×10⁹, from or from about 25×10⁹ to or to about 50×10¹⁰, from or from about 25×10⁹ to or to about 25×10¹⁰, from or from about 25×10⁹ to or to about 1×10¹⁰, from or from about 25×10⁹ to or to about 75×10⁹, from or from about 25×10⁹ to or to about 50×10⁹, from or from about 50×10⁹ to or to about 50×10¹⁰, from or from about 50×10⁹ to or to about 25×10¹⁰, from or from about 50×10⁹ to or to about 1×10¹⁰, from or from about 50×10⁹ to or to about 75×10⁹, from or from about 75×10⁹ to or to about 50×10¹⁰, from or from about 75×10⁹ to or to about 25×10¹⁰, from or from about 75×10⁹ to or to about 1×10¹⁰, from or from about 1×10¹⁰ to or to about 50×10¹⁰, from or from about 1×10¹⁰ to or to about 25×10¹⁰, or from or from about 25×10¹⁰ to or to about 50×10¹⁰ cells, e.g., e.g., MCB cells or infusion-ready drug product cells.

In some embodiments, the expansion yields from or from about 60 to or to about 100 vials, each comprising from or from about 600 million to or to about 1 billion cells, e.g., MCB cells or infusion-ready drug product cells. In some embodiments, the expansion yields 80 or about 80 vials, each comprising or consisting of 800 million or about 800 million cells, e.g., MCB cells or infusion-ready drug product cells.

In some embodiments, the expansion yields from or from about a 100 to or to about a 500 fold increase in the number of cells, e.g., the number of MCB cells relative to the number of cells, e.g., NK cells, in the natural killer cell source. In some embodiments, the expansion yields from or from about a 100 to or to about a 500, from or from about a 100 to or to about a 400, from or from about a 100 to or to about a 300, from or from about a 100 to or to about a 200, from or from about a 200 to or to about a 450, from or from about a 200 to or to about a 400, from or from about a 100 to or to about a 350, from or from about a 200 to or to about a 300, from or from about a 200 to or to about a 250, from or from about a 250 to or to about a 500, from or from about a 250 to or to about a 450, from or from about a 200 to or to about a 400, from or from about a 250 to or to about a 350, from or from about a 250 to or to about a 300, from or from about a 300 to or to about a 500, from or from about a 300 to or to about a 450, from or from about a 300 to or to about a 400, from or from about a 300 to or to about a 350, from or from about a 350 to or to about a 500, from or from about a 350 to or to about a 450, from or from about a 350 to or to about a 400 fold increase in the number of cells, e.g., the number of MCB cells relative to the number of cells, e.g., NK cells, in the natural killer cell source.

In some embodiments, the expansion yields from or from about a 100 to or to about a 70,000 fold increase in the number of cells, e.g., the number of MCB cells relative to the number of cells, e.g., NK cells, in the natural killer cell source. In some embodiments, the expansion yields at least a 10,000 fold, e.g., 15,000 fold, 20,000 fold, 25,000 fold, 30,000 fold, 35,000 fold, 40,000 fold, 45,000 fold, 50,000 fold, 55,000 fold, 60,000 fold, 65,000 fold, or 70,000 fold increase in the number of cells, e.g., the number of MCB cells relative to the number of cells, e.g., NK cells, in the natural killer cell source.

In some embodiments, the co-culture of the MCB cells and feeder cells yields from or from about 500 million to or to about 1.5 billion cells, e.g., NK cells suitable for use in an MCB and/or in an infusion-ready drug product. In some embodiments, the co-culture of the MCB cells and feeder cells yields from or from about 500 million to or to about 1.5 billion, from or from about 500 million to or to about 1.25 billion, from or from about 500 million to or to about 1 billion, from or from about 500 million to or to about 750 million, from or from about 750 million to or to about 1.5 billion, from or from about 500 million to or to about 1.25 billion, from or from about 750 million to or to about 1 billion, from or from about 1 billion to or to about 1.5 billion, from or from about 1 billion to or to about 1.25 billion, or from or from about 1.25 billion to or to about 1.5 billion cells, e.g., NK cells suitable for use in an MCB and/or an infusion-ready drug product.

In some embodiments, the co-culture of the MCB cells and feeder cells yields from or from about 50 to or to about 150 vials of cells, e.g., infusion-ready drug product cells, each comprising from or from about 750 million to or to about 1.25 billion cells, e.g., NK cells suitable for use in an MCB and/or an infusion-ready drug product. In some embodiments, the co-culture of the MCB cells and feeder cells yields 100 or about 100 vials, each comprising or consisting of 1 billion or about 1 billion cells, e.g., NK cells suitable for use in an MCB and/or an infusion-ready drug product.

In some embodiments, the expansion yields from or from about a 100 to or to about a 500 fold increase in the number of cells, e.g., the number of NK cells suitable for use in an MCB and/or an infusion-ready drug product relative to the number of starting MCB cells. In some embodiments, the expansion yields from or from about a 100 to or to about a 500, from or from about a 100 to or to about a 400, from or from about a 100 to or to about a 300, from or from about a 100 to or to about a 200, from or from about a 200 to or to about a 450, from or from about a 200 to or to about a 400, from or from about a 100 to or to about a 350, from or from about a 200 to or to about a 300, from or from about a 200 to or to about a 250, from or from about a 250 to or to about a 500, from or from about a 250 to or to about a 450, from or from about a 200 to or to about a 400, from or from about a 250 to or to about a 350, from or from about a 250 to or to about a 300, from or from about a 300 to or to about a 500, from or from about a 300 to or to about a 450, from or from about a 300 to or to about a 400, from or from about a 300 to or to about a 350, from or from about a 350 to or to about a 500, from or from about a 350 to or to about a 450, from or from about a 350 to or to about a 400 fold increase in the number of cells, e.g., the number of NK cells suitable for use in an MCB and/or an infusion-ready drug product relative to the number of starting MCB cells.

In some embodiments, the expansion yields from or from about a 100 to or to about a 70,000 fold increase in the number of cells, e.g., the number of NK cells suitable for use in an MCB and/or an infusion-ready drug product relative to the number of starting MCB cells. In some embodiments, the expansion yields at least a 10,000 fold, e.g., 15,000 fold, 20,000 fold, 25,000 fold, 30,000 fold, 35,000 fold, 40,000 fold, 45,000 fold, 50,000 fold, 55,000 fold, 60,000 fold, 65,000 fold, or 70,000 fold increase in the number of cells, e.g., the number of NK cells suitable for use in an MCB and/or an infusion-ready drug product relative to the number of starting MCB cells.

In embodiments where the cells are engineered during expansion and stimulation, as described herein, not all of the expanded and stimulated cells will necessarily be engineered successfully, e.g., transduced successfully, e.g., transduced successfully with a vector comprising a heterologous protein, e.g., a heterologous protein comprising a CAR and/or IL-15 as described herein. Thus, the methods described herein can further comprise sorting engineered cells, e.g., engineered cells described herein, away from non-engineered cells.

In some embodiments, the engineered cells, e.g., transduced cells, are sorted from the non-engineered cells, e.g., the non-transduced cells using a reagent specific to an antigen of the engineered cells, e.g., an antibody that targets an antigen of the engineered cells but not the non-engineered cells. In some embodiments, the antigen of the engineered cells is a component of a CAR, e.g., a CAR described herein.

Systems for antigen-based cell separation of cells are available commercially, e.g., the CliniMACS® sorting system (Miltenyi Biotec).

In some embodiments, the engineered cells, e.g., transduced cells, are sorted from the non-engineered cells, e.g., the non-transduced cells using flow cytometry.

In some embodiments, the sorted engineered cells are used as an MCB. In some embodiments, the sorted engineered cells are used as a component in an infusion-ready drug product.

In some embodiments, the engineered cells, e.g., transduced cells, are sorted from the non-engineered cells, e.g., the non-transduced cells using a microfluidic cell sorting method. Microfluidic cell sorting methods are described, for example, in Dalili et al., “A Review of Sorting, Separation and Isolation of Cells and Microbeads for Biomedical Applications: Microfluidic Approaches,” Analyst 144:87 (2019).

In some embodiments, from or from about 1% to or to about 99% of the expanded and stimulated cells are engineered successfully, e.g., transduced successfully, e.g., transduced successfully with a vector comprising a heterologous protein, e.g., a heterologous protein comprising a CAR and/or IL-15 as described herein. In some embodiments, from or from about 1% to or to about 90%, from or from about 1% to or to about 80%, from or from about 1% to or to about 70%, from or from about 1% to or to about 60%, from or from about 1% to or to about 50%, from or from about 1% to or to about 40%, from or from about 1% to or to about 30%, from or from about 1% to or to about 20%, from or from about 1% to or to about 10%, from or from about 1% to or to about 5%, from or from about 5% to or to about 99%, from or from about 5% to or to about 90%, from or from about 5% to or to about 80%, from or from about 5% to or to about 70%, from or from about 5% to or to about 60%, from or from about 5% to or to about 50%, from or from about 5% to or to about 40%, from or from about 5% to or to about 30%, from or from about 5% to or to about 20%, from or from about 5% to or to about 10%, from or from about 10% to or to about 99%, from or from about 10% to or to about 90%, from or from about 10% to or to about 80%, from or from about 10% to or to about 70%, from or from about 10% to or to about 60%, from or from about 10% to or to about 50%, from or from about 10% to or to about 40%, from or from about 10% to or to about 30%, from or from about 10% to or to about 20%, from or from about 20% to or to about 99%, from or from about 20% to or to about 90%, from or from about 20% to or to about 80%, from or from about 20% to or to about 70%, from or from about 20% to or to about 60%, from or from about 20% to or to about 50%, from or from about 20% to or to about 40%, from or from about 20% to or to about 30%, from or from about 30% to or to about 99%, from or from about 30% to or to about 90%, from or from about 30% to or to about 80%, from or from about 30% to or to about 70%, from or from about 30% to or to about 60%, from or from about 30% to or to about 50%, from or from about 30% to or to about 40%, from or from about 40% to or to about 99%, from or from about 40% to or to about 90%, from or from about 40% to or to about 80%, from or from about 40% to or to about 70%, from or from about 40% to or to about 70%, from or from about 40% to or to about 60%, from or from about 40% to or to about 50%, from or from about 50% to or to about 99%, from or from about 50% to or to about 90%, from or from about 50% to or to about 80%, from or from about 50% to or to about 70%, from or from about 50% to or to about 60%, from or from about 60% to or to about 99%, from or from about 60% to or to about 90%, from or from about 60% to or to about 80%, from or from about 60% to or to about 70%, from or from about 70% to or to about 99%, from or from about 70% to or to about 90%, from or from about 70% to or to about 80%, from or from about 80% to or to about 99%, from or from about 80% to or to about 90%, or from or from about 90% to or to about 99% of the expanded and stimulated cells are engineered successfully, e.g., transduced successfully, e.g., transduced successfully with a vector comprising a heterologous protein, e.g., a heterologous protein comprising a CAR and/or IL-15 as described herein.

In some embodiments, frozen cells of a first or second MCB are thawed and cultured. In some embodiments, a single vial of frozen cells of the first or second MCB e.g., a single vial comprising 800 or about 800 million cells, e.g., first or second MCB cells, are thawed and cultured. In some embodiments, the frozen first or second MCB cells are cultured with additional feeder cells to produce cells suitable for use either as a second or third MCB or in an infusion-ready drug product. In some embodiments, the cells from the co-culture of the first or second MCB are harvested and frozen.

In some embodiments, the cells from the co-culture of the natural killer cell source, a first MCB, or a second MCB are harvested, and frozen in a cryopreservation composition, e.g., a cryopreservation composition described herein. In some embodiments, the cells are washed after harvesting. Thus, provided herein is a pharmaceutical composition comprising activated and stimulated NK cells, e.g., activated and stimulated NK cells produced by the methods described herein, e.g., harvested and washed activated and stimulated NK cells produced by the methods described herein and a cryopreservation composition, e.g., a cryopreservation composition described herein.

In some embodiments, the cells are mixed with a cryopreservation composition, e.g., as described herein, before freezing. In some embodiments, the cells are frozen in cryobags. In some embodiments, the cells are frozen in cryovials.

In some embodiments, the method further comprises isolating NK cells from the population of expanded and stimulated NK cells.

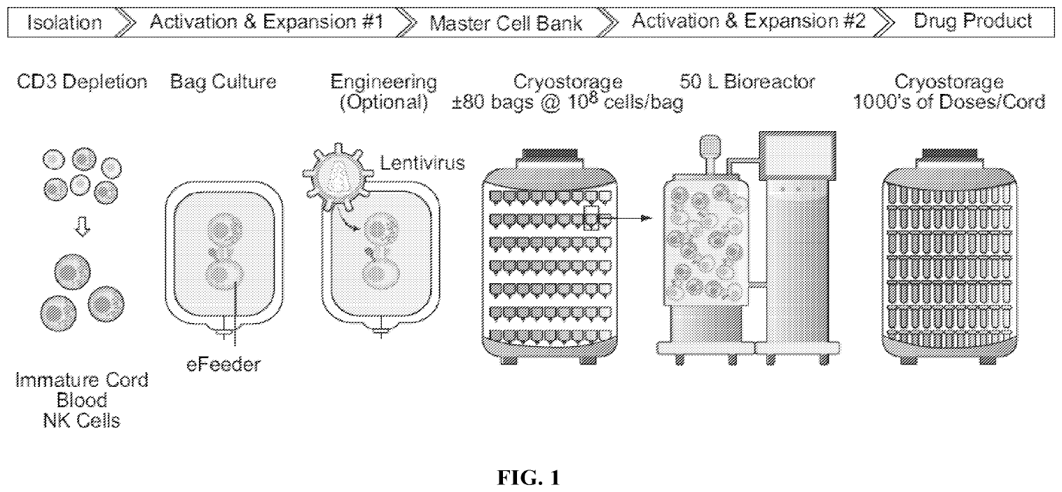

An exemplary process for expanding and stimulating NK cells is shown in FIG. 1. In some embodiments, the natural killer cells are not genetically engineered.

### E. Properties of Expanded and Stimulated NK Cells

After having been ex vivo expanded and stimulated, e.g., as described herein, the expanded and stimulated NK cell populations not only have a number/density (e.g., as described above) that could not occur naturally in the human body, but they also differ in their phenotypic characteristics, (e.g., gene expression and/or surface protein expression) with the starting source material or other naturally occurring populations of NK cells.

In some cases, the starting NK cell source is a sample derived from a single individual, e.g., a single cord blood unit that has not been ex vivo expanded. Therefore, in some cases, the expanded and stimulated NK cells share a common lineage, i.e., they all result from expansion of the starting NK cell source, and, therefore, share a genotype via clonal expansion of a population of cells that are, themselves, from a single organism. Yet, they could not occur naturally at the density achieved with ex vivo expansion and also differ in phenotypic characteristics from the starting NK cell source.

In some cases, the population of expanded and stimulated NK cells comprises at least 100 million expanded natural killer cells, e.g., 200 million, 250 million, 300 million, 400 million, 500 million, 600 million, 700 million, 750 million, 800 million, 900 million, 1 billion, 2 billion, 3 billion, 4 billion, 5 billion, 6 billion, 7 billion, 8 billion, 9 billion, 10 billion, 15 billion, 20 billion, 25 billion, 50 billion, 75 billion, 80 billion, 9-billion, 100 billion, 200 billion, 250 billion, 300 billion, 400 billion, 500 billion, 600 billion, 700 billion, 800 billion, 900 billion, 1 trillion, 2 trillion, 3 trillion, 4 trillion, 5 trillion, 6 trillion, 7 trillion, 8 trillion, 9 trillion, or 10 trillion expanded natural killer cells.

In some embodiments, the expanded and stimulated NK cells comprise at least 80%, e.g., at least 90%, at least 95%, at least 99%, or 100% CD56+CD3-cells.

In some embodiments, the expanded and stimulated NK cells are not genetically engineered.

In some embodiments, the expanded and stimulated NK cells do not comprise a CD16 transgene.

In some embodiments, the expanded and stimulated NK cells do not express an exogenous CD16 protein.

The expanded and stimulated NK cells can be characterized, for example, by surface expression, e.g., of one or more of CD16, CD56, CD3, CD38, CD14, CD19, NKG2D, NKp46, NKp30, DNAM-1, and NKp44.

The surface protein expression levels stated herein, in some cases are achieved without positive selection on the particular surface protein referenced. For example, in some cases, the NK cell source, e.g., a single cord unit, comprises both the KIR B allele of the KIR receptor family and the 158 V/V variant of CD16 and is + enriched and CD3 (+) depleted, e.g., by gating on CD56+CD3-expression or using magnetic beads, including immunoaffinity magnetic beads (for example, a CliniMACS Prodigy® system), but no other surface protein expression selection is carried out during expansion and stimulation.

In some embodiments, the expanded and stimulated NK cells, e.g., from expansion and stimulation of a single cord blood unit, e.g., as described above, comprise at least 60%, e.g., at least 70%, at least 80%, at least 90% at least 95%, at least 99%, or 100% NKG2D+ cells.

In some embodiments, the expanded and stimulated NK cells, e.g., from expansion and stimulation of a single cord blood unit, e.g., as described above, comprise at least 60%, e.g., at least 70%, at least 80%, at least 90% at least 95%, at least 99%, or 100% NKp46+ cells.

In some embodiments, the expanded and stimulated NK cells, e.g., from expansion and stimulation of a single cord blood unit, e.g., as described above, comprise at least 60%, e.g., at least 70%, at least 80%, at least 90% at least 95%, at least 99%, or 100% NKp30+ cells.

In some embodiments, the expanded and stimulated NK cells, e.g., from expansion and stimulation of a single cord blood unit, e.g., as described above, comprise at least 60%, e.g., at least 70%, at least 80%, at least 90% at least 95%, at least 99%, or 100% DNAM-1+ cells.

In some embodiments, the expanded and stimulated NK cells, e.g., from expansion and stimulation of a single cord blood unit, e.g., as described above, comprise at least 60%, e.g., at least 70%, at least 80%, at least 90% at least 95%, at least 99%, or 100% NKp44+ cells.

In some embodiments, the expanded and stimulated NK cells, e.g., from expansion and stimulation of a single cord blood unit, e.g., as described above, comprise at least 60%, e.g., at least 70%, at least 80%, at least 90% at least 95%, at least 99%, or 100% CD94+ (KLRD1) cells.

In some embodiments, the expanded and stimulated NK cells, e.g., from expansion and stimulation of a single cord blood unit, e.g., as described above, comprises less than or equal to 20%, e.g., less than or equal to 10%, less than or equal to 5%, less than or equal to 1% or 0% CD3+ cells.

In some embodiments, the expanded and stimulated NK cells, e.g., from expansion and stimulation of a single cord blood unit, e.g., as described above, comprises less than or equal to 20%, e.g., less than or equal to 10%, less than or equal to 5%, less than or equal to 1% or 0% CD14+ cells.

In some embodiments, the expanded and stimulated NK cells, e.g., from expansion and stimulation of a single cord blood unit, e.g., as described above, comprises less than or equal to 20%, e.g., less than or equal to 10%, less than or equal to 5%, less than or equal to 1% or 0% CD19+ cells.

In some embodiments, the expanded and stimulated NK cells, e.g., from expansion and stimulation of a single cord blood unit, e.g., as described above, comprises less than or equal to 20%, e.g., less than or equal to 10%, less than or equal to 5%, less than or equal to 1% or 0% CXCR+ cells.

In some embodiments, the expanded and stimulated NK cells, e.g., from expansion and stimulation of a single cord blood unit, e.g., as described above, comprises less than or equal to 20%, e.g., less than or equal to 10%, less than or equal to 5%, less than or equal to 1% or 0% CD122+ (IL2RB) cells.

As described herein, the inventors have demonstrated that, surprisingly, the NK cells expanded and stimulated by the methods described herein express CD16 at high levels throughout the expansion and stimulation process, resulting in a cell population with high CD16 expression. The high expression of CD16 obviates the need for engineering the expanded cells to express CD16, which is important for initiating ADCC, and, therefore, a surprising and unexpected benefit of the expansion and stimulation methods described herein. Thus, in some embodiments, the expanded and stimulated NK cells, e.g., from expansion and stimulation of a single cord blood unit, e.g., as described above, comprise 50% or more, e.g., 55%, 60%, 65%, 70%, 75%, 80%, 85%, 90%, or 95% CD16+NK cells.

In some embodiments, the expanded and stimulated NK cells, e.g., from expansion and stimulation of a single cord blood unit, e.g., as described above, comprises both the KIR B allele of the KIR receptor family and the 158 V/V variant of CD16 and comprise 50% or more, e.g., 55%, 60%, 65%, 70%, 75%, 80%, 85%, 90%, or 95% CD16+NK cells.

In some embodiments, the percentage of expanded and stimulated NK cells, e.g., from expansion and stimulation of a single cord blood unit, e.g., as described above, expressing CD16 is the same or higher than the percentage of natural killer cells in the seed cells from umbilical cord blood.

In some embodiments, the percentage of expanded and stimulated NK cells, e.g., from expansion and stimulation of a single cord blood unit, e.g., as described above, expressing NKG2D is the same or higher than the percentage of natural killer cells in the seed cells from umbilical cord blood.

In some embodiments, the percentage of expanded and stimulated NK cells, e.g., from expansion and stimulation of a single cord blood unit, e.g., as described above, expressing NKp30 is the same or higher than the percentage of natural killer cells in the seed cells from umbilical cord blood.

In some embodiments, the percentage of expanded and stimulated NK cells, e.g., from expansion and stimulation of a single cord blood unit, e.g., as described above, expressing DNAM-1 is the same or higher than the percentage of natural killer cells in the seed cells from umbilical cord blood.

In some embodiments, the percentage of expanded and stimulated NK cells, e.g., from expansion and stimulation of a single cord blood unit, e.g., as described above, expressing NKp44 is the same or higher than the percentage of natural killer cells in the seed cells from umbilical cord blood.

In some embodiments, the percentage of expanded and stimulated NK cells, e.g., from expansion and stimulation of a single cord blood unit, e.g., as described above, expressing NKp46 is the same or higher than the percentage of natural killer cells in the seed cells from umbilical cord blood.

As described herein, the inventors have also demonstrated that, surprisingly, the NK cells expanded and stimulated by the methods described herein express CD38 at low levels. CD38 is an effective target for certain cancer therapies (e.g., multiple myeloma and acute myeloid leukemia). See. e.g., Jiao et al., “CD38: Targeted Therapy in Multiple Myeloma and Therapeutic Potential for Solid Cancerrs,” Expert Opinion on Investigational Drugs 29 (11): 1295-1308 (2020). Yet, when an anti-CD38 antibody is administered with NK cells, because NK cells naturally express CD38, they are at risk for increased fratricide. The NK cells expanded and stimulated by the methods described herein, however, express low levels of CD38 and, therefore, overcome the anticipated fratricide. While other groups have resorted to engineering methods such as genome editing to reduce CD38 expression (see. e.g., Gurney et al., “CD38 Knockout Natural Killer Cells Expressing an Affinity Optimized CD38 Chimeric Antigen Receptor Successfully Target Acute Myeloid Leukemia with Reduced Effector Cell Fratricide,” Haematologica doi: 10.3324/haematol.2020.271908 (2020), the NK cells expanded and stimulated by the methods described herein express low levels of CD38 without the need for genetic engineering, which provides a surprising and unexpected benefits, e.g., for treating CD38+ cancers with the NK cells expanded and stimulated as described herein, e.g., in combination with a CD38 antibody.

Thus, in some embodiments, the expanded and stimulated NK cells, e.g., from expansion and stimulation of a single cord blood unit, e.g., as described above, comprise less than or equal to 80% CD38+ cells, e.g., less than or equal to 75%, 70%, 65%, 60%, 55%, 50%, 45%, 40%, 35%, 30%, 25%, or 20% CD38+ cells.

In some embodiments, the expanded and stimulated NK cells, e.g., from expansion and stimulation of a single cord blood unit, e.g., as described above, comprises both the KIR B allele of the KIR receptor family and the 158 V/V variant of CD16 and comprise less than or equal to 80% CD38+ cells, e.g., less than or equal to 75%, 70%, 65%, 60%, 55%, 50%, 45%, 40%, 35%, 30%, 25%, or 20% CD38+ cells.

In some embodiments, the expanded and stimulated NK cells, e.g., from expansion and stimulation of a single cord blood unit, e.g., as described above, comprises both the KIR B allele of the KIR receptor family and the 158 V/V variant of CD16 and comprise less than or equal to 80% CD38+ cells, e.g., less than or equal to 75%, 70%, 65%, 60%, 55%, 50%, 45%, 40%, 35%, 30%, 25%, or 20% CD38+ cells, and 50% or more, e.g., 55%, 60%, 65%, 70%, 75%, 80%, 85%, 90%, or 95% CD16+NK cells.

In some embodiments, the expanded and stimulated NK cells, e.g., from expansion and stimulation of a single cord blood unit, e.g., as described above, comprises both the KIR B allele of the KIR receptor family and the 158 V/V variant of CD16 and comprise: i) 50% or more, e.g., 55%, 60%, 65%, 70%, 75%, 80%, 85%, 90%, or 95% CD16+NK cells: and/or ii) less than or equal to 80% CD38+ cells, e.g., less than or equal to 75%, 70%, 65%, 60%, 55%, 50%, 45%, 40%, 35%, 30%, 25%, or 20% CD38+ cells; and/or iii) at least 60%, e.g., at least 70%, at least 80%, at least 90% at least 95%, at least 99%, or 100% NKG2D+ cells; and/or iv) at least 60%, e.g., at least 70%, at least 80%, at least 90% at least 95%, at least 99%, or 100% NKp46+ cells; and/or v) at least 60%, e.g., at least 70%, at least 80%, at least 90% at least 95%, at least 99%, or 100% NKp30+ cells; and/or vi) at least 60%, e.g., at least 70%, at least 80%, at least 90% at least 95%, at least 99%, or 100% DNAM-1+ cells; and/or vii) at least 60%, e.g., at least 70%, at least 80%, at least 90% at least 95%, at least 99%, or 100% NKp44+ cells; and/or viii) at least 60%, e.g., at least 70%, at least 80%, at least 90% at least 95%, at least 99%, or 100% CD94+ (KLRD1) cells; and/or ix) less than or equal to 20%, e.g., less than or equal to 10%, less than or equal to 5%, less than or equal to 1% or 0% CD3+ cells; and/or x) less than or equal to 20%, e.g., less than or equal to 10%, less than or equal to 5%, less than or equal to 1% or 0% CD14+ cells; and/or xi) less than or equal to 20%, e.g., less than or equal to 10%, less than or equal to 5%, less than or equal to 1% or 0% CD19+ cells; and/or xii) less than or equal to 20%, e.g., less than or equal to 10%, less than or equal to 5%, less than or equal to 1% or 0% CXCR+ cells; and/or xiii) less than or equal to 20%, e.g., less than or equal to 10%, less than or equal to 5%, less than or equal to 1% or 0% CD122+ (IL2RB) cells.

In some embodiments, feeder cells do not persist in the expanded and stimulated NK cells, though, residual signature of the feeder cells may be detected, for example, by the presence of residual cells (e.g., by detecting cells with a particular surface protein expression) or residual nucleic acid and/or proteins that are expressed by the feeder cells.

For example, in some cases, the methods described herein include expanding and stimulating natural killer cells using engineered feeder cells, e.g., eHuT-78 feeder cells described above, which are engineered to express sequences that are not expressed by cells in the natural killer cell source, including the natural killer cells. For example, the engineered feeder cells can be engineered to express at least one gene selected from the group consisting of 4-1BBL (UniProtKB P41273, SEQ ID NO: 1), membrane bound IL-21 (SEQ ID NO: 2), and mutant TNFalpha (SEQ ID NO: 3) (“eHut-78 cells”), or variants thereof.

While these feeder cells may not persist in the expanded and stimulated NK cells, the expanded and stimulated NK cells may retain detectable residual amounts of cells, proteins, and/or nucleic acids from the feeder cells. Thus, their residual presence in the expanded and stimulated NK cells may be detected, for example, by detecting the cells themselves (e.g., by flow cytometry), proteins that they express, and/or nucleic acids that they express.

Thus, also described herein is a population of expanded and stimulated NK cells comprising residual feeder cells (live cells or dead cells) or residual feeder cell cellular impurities (e.g., residual feeder cell proteins or portions thereof, and/or genetic material such as a nucleic acid or portion thereof). In some cases, the expanded and stimulated NK cells comprise more than 0% and, but 0.3% or less residual feeder cells, e.g., eHuT-78 feeder cells.

In some cases, the expanded and stimulated NK cells comprise residual feeder cell nucleic acids, e.g., encoding residual 4-1BBL (UniProtKB P41273, SEQ ID NO: 1), membrane bound IL-21 (SEQ ID NO: 2), and/or mutant TNFalpha (SEQ ID NO: 3) or portion(s) thereof. In some cases, the membrane bound IL-21 comprises a CD8 transmembrane domain

In some cases, the expanded and stimulated NK cells comprise a % residual feeder cells of more than 0% and less than or equal to 0.2%, as measured, e.g., by the relative proportion of a feeder cell specific protein or nucleic acid sequence (that is, a protein or nucleic acid sequence not expressed by the natural killer cells) in the sample. For example, by qPCR, e.g., as described herein.

In some embodiments, the residual feeder cells are CD4 (+) T cells. In some embodiments, the residual feeder cells are engineered CD4 (+) T cells. In some embodiments, the residual feeder cell cells are engineered to express at least one gene selected from the group consisting of 4-1BBL (UniProtKB P41273, SEQ ID NO: 1), membrane bound IL-21 (SEQ ID NO: 2), and mutant TNFalpha (SEQ ID NO: 3) (“eHut-78 cells”), or variants thereof. Thus, in some cases, the feeder cell specific protein is 4-1BBL (UniProtKB P41273, SEQ ID NO: 1), membrane bound IL-21 (SEQ ID NO: 2), and/or mutant TNFalpha (SEQ ID NO: 3). And, therefore, the feeder cell specific nucleic acid is a nucleic acid encoding 4-1BBL (UniProtKB P41273, SEQ ID NO: 1), membrane bound IL-21 (SEQ ID NO: 2), and/or mutant TNFalpha (SEQ ID NO: 3), or portion thereof. In some cases, the membrane bound IL-21 comprises a CD8 transmembrane domain.

A wide variety of different methods can be used to analyze and detect the presence of nucleic acids or protein gene products in a biological sample. As used herein, “detecting” can refer to a method used to discover, determine, or confirm the existence or presence of a compound and/or substance (e.g., a cell, a protein and/or a nucleic acid). In some embodiments, a detecting method can be used to detect a protein. In some embodiments, detecting can include chemiluminescence or fluorescence techniques. In some embodiments, detecting can include immunological-based methods (e.g., quantitative enzyme-linked immunosorbent assays (ELISA), Western blotting, or dot blotting) wherein antibodies are used to react specifically with entire proteins or specific epitopes of a protein. In some embodiments, detecting can include immunoprecipitation of the protein (Jungblut et al., J Biotechnol. 31: 41 (2-3): 111-20 (1995); Franco et al., Eur J Morphol. 39 (1): 3-25 (2001)). In some embodiments, a detecting method can be used to detect a nucleic acid (e.g., DNA and/or RNA). In some embodiments, detecting can include Northern blot analysis, nuclease protection assays (NPA), in situ hybridization, or reverse transcription-polymerase chain reaction (RT-PCR) (Raj et al., Nat. Methods 5, 877-879 (2008): Jin et al., J Clin Lab Anal. 11 (1): 2-9 (1997): Ahmed, J Environ Sci Health C Environ Carcinog Ecotoxicol Rev. 20 (2): 77-116 (2002)).

Thus, also described herein, are methods for detecting a population of expanded and stimulated NK cells, e.g., expanded and stimulated using the methods described herein, that have been co-cultured with engineered feeder cells, e.g., eHuT-78 feeder cells described herein.

### II. CRYOPRESERVATION

### A. Cryopreservation Compositions

Provided herein are cryopreservation compositions, e.g., cryopreservation compositions suitable for intravenous administration, e.g., intravenous administration of NK cells, e.g., the NK cells described herein. In some embodiments, a pharmaceutical composition comprises the cryopreservation composition and cells, e.g., the NK cells described herein.

### 1. Albumin

In some embodiments, the cryopreservation composition comprises albumin protein, e.g., human albumin protein (UniProtKB Accession P0278, SEQ ID NO: 5) or variant thereof. In some embodiments, the cryopreservation composition comprises an ortholog of an albumin protein, e.g., human albumin protein, or variant thereof. In some embodiments, the cryopreservation composition comprises a biologically active portion of an albumin protein, e.g., human albumin, or variant thereof.

In some embodiments, the albumin, e.g., human albumin, is provided as a solution, also referred to herein as an albumin solution or a human albumin solution. Thus, in some embodiments, the cryopreservation composition is or comprises an albumin solution, e.g., a human albumin solution. In some embodiments, the albumin solution is a serum-free albumin solution.

In some embodiments, the albumin solution is suitable for intravenous use.

In some embodiments, the albumin solution comprises from or from about 40 to or to about 200 g/L albumin. In some embodiments, the albumin solution comprises from or from about 40 to or to about 50 g/L albumin, e.g., human albumin. In some embodiments, the albumin solution comprises about 200 g/L albumin, e.g., human albumin. In some embodiments, the albumin solution comprises 200 g/L albumin, e.g., human albumin.

In some embodiments, the albumin solution comprises a protein composition, of which 95% or more is albumin protein, e.g., human albumin protein. In some embodiments, 96%, 97%, 98%, or 99% or more of the protein is albumin, e.g., human albumin.

In some embodiments, the albumin solution further comprises sodium. In some embodiments, the albumin solution comprises from or from about 100 to or to about 200 mmol sodium. In some embodiments, the albumin solution comprises from or from about 130 to or to about 160 mmol sodium.

In some embodiments, the albumin solution further comprises potassium. In some embodiments, the albumin solution comprises 3 mmol or less potassium. In some embodiments, the albumin solution further comprises 2 mmol or less potassium.

In some embodiments, the albumin solution further comprises one or more stabilizers. In some embodiments, the stabilizer(s) are selected from the group consisting of sodium caprylate, caprylic acid, (2S)-2-acetamido-3-(1H-indol-3-yl) propanoic acid (also referred to as acetyl tryptophan, N-Acetyl-L-tryptophan and Acetyl-L-tryptophan), 2-acetamido-3-(1H-indol-3-yl) propanoic acid (also referred to as N-acetyltryptophan, DL-Acetyltroptohan and N-Acetyl-DL-tryptophan). In some embodiments, the solution comprises less than 0.1 mmol of each of the one or more stabilizers per gram of protein in the solution. In some embodiments, the solution comprises from or from about 0.05 to or to about 0.1, e.g., from or from about 0.064 to or to about 0.096 mmol of each of the stabilizers per gram of protein in the solution. In some embodiments, the solution comprises less than 0.1 mmol of total stabilizer per gram of protein in the solution. In some embodiments, the solution comprises from or from about 0.05 to or to about 0.1, e.g., from or from about 0.064 to or to about 0.096 mmol of total stabilizer per gram of protein in the solution.

In some embodiments, the albumin solution consists of a protein composition, of which 95% or more is albumin protein, sodium, potassium, and one or more stabilizers selected from the group consisting of sodium caprylate, caprylic acid, (2S)-2-acetamido-3-(1H-indol-3-yl) propanoic acid (also referred to as acetyl tryptophan, N-Acetyl-L-tryptophan and Acetyl-L-tryptophan), 2-acetamido-3-(1H-indol-3-yl) propanoic acid (also referred to as N-acetyltryptophan, DL-Acetyltroptohan and N-Acetyl-DL-tryptophan) in water.

In some embodiments, the cryopreservation composition comprises from or from about 10% v/v to or to about 50% v/v of an albumin solution, e.g., an albumin solution described herein. In some embodiments, the cryopreservation composition comprises from or from about 10% to or to about 50%, from or from about 10% to or to about 45%, from or from about 10% to or to about 40%, from or from about 10% to or to about 35%, from or from about 10% to or to about 30%, from or from about 10% to or to about 25%, from or from about 10% to or to about 20%, from or from about 10% to or to about 15%, from or from about 15% to or to about 50%, from or from about 15% to or to about 45%, from or from about 15% to or to about 40%, from or from about 15% to or to about 35%, from or from about 15% to or to about 30%, from or from about 15% to or to about 25%, from or from about 15% to or to about 20%, from or from about 20% to or to about 50%, from or from about 20% to or to about 45%, from or from about 20% to or to about 40%, from or from about 20% to or to about 35%, from or from about 20% to or to about 30%, from or from about 20% to or to about 25%, from or from about 25% to or to about 50%, from or from about 25% to or to about 45%, from or from about 25% to or to about 40%, from or from about 25% to or to about 35%, from or from about 25% to or to about 30%, from or from about 30% to or to about 50%, from or from about 30% to or to about 45%, from or from about 30% to or to about 40%, from or from about 30% to or to about 35%, from or from about 35% to or to about 50%, from or from about 35% to or to about 45%, from or from about 35% to or to about 40%, from or from about 40% to or to about 50%, from or from about 40% to or to about 45%, or from or from about 45% to or to about 50% v/v of an albumin solution described herein. In some embodiments, the cryopreservation composition comprises about 10%, about 15%, about 20%, about 25%, about 30%, about 35%, about 40%, about 45%, or about 50% v/v of an albumin solution described herein. In some embodiments, the cryopreservation composition comprises 10%, 15%, 20%, 25%, 30%, 35%, 40%, 45%, or 50% v/v of an albumin solution described herein.

In some embodiments, the cryopreservation composition comprises from or from about 20 to or to about 100 g/L albumin, e.g., human albumin. In some embodiments, the cryopreservation composition comprises from or from about 20 to or to about 100, from or from about 20 to or to about 90, from or from about 20 to or to about 80, from or from about 20 to or to about 70, from or from about 20 to or to about 60, from or from about 20 to or to about 50, from or from about 20 to or to about 40, from or from about 20 to or to about 30, from or from about 30 to or to about 100, from or from about 30 to or to about 90, from or from about 30 to or to about 80, from or from about 30 to or to about 70, from or from about 30 to or to about 60, from or from about 30 to or to about 50, from or from about 30 to or to about 40, from or from about 40 to or to about 100, from or from about 40 to or to about 90, from or from about 40 to or to about 80, from or from about 40 to or to about 70, from or from about 40 to or to about 60, from or from about 40 to or to about 50, from or from about 50 to or to about 100, from or from about 50 to or to about 90, from or from about 50 to or to about 80, from or from about 50 to or to about 70, from or from about 50 to or to about 60, from or from about 60 to or to about 100, from or from about 60 to or to about 90, from or from about 60 to or to about 80, from or from about 60 to or to about 70, from or from about 70 to or to about 100, from or from about 70 to or to about 90, from or from about 70 to or to about 80, from or from about 80 to or to about 100, from or from about 80 to or to about 90, or from or from about 90 to or to about 100 g/L albumin, e.g., human albumin.

In some embodiments, the cryopreservation composition comprises 20 g/L albumin, e.g., human albumin. In some embodiments, the cryopreservation composition comprises 40 g/L albumin, e.g., human albumin. In some embodiments, the cryopreservation composition comprises 70 g/L albumin, e.g., human albumin. In some embodiments, the cryopreservation composition comprises 100 g/L albumin, e.g., human albumin.

In some embodiments, the cryopreservation composition comprises about 20 g/L albumin, e.g., human albumin. In some embodiments, the cryopreservation composition comprises about 40 g/L albumin, e.g., human albumin. In some embodiments, the cryopreservation composition comprises about 70 g/L albumin, e.g., human albumin. In some embodiments, the cryopreservation composition comprises about 100 g/L albumin, e.g., human albumin.

In some embodiments, the cryopreservation composition further comprises a stabilizer, e.g., an albumin stabilizer. In some embodiments, the stabilizer(s) are selected from the group consisting of sodium caprylate, caprylic acid, (2S)-2-acetamido-3-(1H-indol-3-yl) propanoic acid (also referred to as acetyl tryptophan, N-Acetyl-L-tryptophan and Acetyl-L-tryptophan), 2-acetamido-3-(1H-indol-3-yl) propanoic acid (also referred to as N-acetyltryptophan, DL-Acetyltroptohan and N-Acetyl-DL-tryptophan). In some embodiments, the cryopreservation composition comprises less than. 1 mmol of each of the one or more stabilizers per gram of protein, e.g., per gram of albumin protein, in the composition. In some embodiments, the cryopreservation composition comprises from or from about 0.05 to or to about 0.1, e.g., from or from about 0.064 to or to about 0.096 mmol of each of the stabilizers per gram of protein, e.g., per gram of albumin protein in the composition. In some embodiments, the cryopreservation composition comprises less than 0.1 mmol of total stabilizer per gram of protein, e.g., per gram of albumin protein in the cryopreservation composition. In some embodiments, the cryopreservation composition comprises from or from about 0.05 to or to about 0.1, e.g., from or from about 0.064 to or to about 0.096 mmol of total stabilizer per gram of protein, e.g., per gram of albumin protein, in the cryopreservation composition.

### 2. Dextran

In some embodiments, the cryopreservation composition comprises Dextran, or a derivative thereof.

Dextran is a polymer of anhydroglucose composed of approximately 95% α-D-(1-6) linkages (designated (C₆H₁₀O₅)ₙ). Dextran fractions are supplied in molecular weights of from about 1,000 Daltons to about 2,000,000 Daltons. They are designated by number (Dextran X), e.g., Dextran 1, Dextran 10, Dextran 40, Dextran 70, and so on, where X corresponds to the mean molecular weight divided by 1,000 Daltons. So, for example, Dextran 40 has an average molecular weight of or about 40,000 Daltons.

In some embodiments, the average molecular weight of the dextran is from or from about 1,000 Daltons to or to about 2,000,000 Daltons. In some embodiments, the average molecular weight of the dextran is or is about 40,000 Daltons. In some embodiments, the average molecular weight of the dextran is or is about 70,000 Daltons.

In some embodiments, the dextran is selected from the group consisting of Dextran 40, Dextran 70, and combinations thereof. In some embodiments, the dextran is Dextran 40.

In some embodiments, the dextran, e.g., Dextran 40, is provided as a solution, also referred to herein as a dextran solution or a Dextran 40 solution. Thus, in some embodiments, the composition comprises a dextran solution, e.g., a Dextran 40 solution.

In some embodiments, the dextran solution is suitable for intravenous use.

In some embodiments, the dextran solution comprises about 5% to about 50% w/w dextran, e.g., Dextran 40. In some embodiments, the dextran solution comprises from or from about 5% to or to about 50%, from or from about 5% to or to about 45%, from or from about 5% to or to about 40%, from or from about 5% to or to about 35%, from or from about 5% to or to about 30%, from or from about 5% to or to about 25%, from or from about 5% to or to about 20%, from or from about 5% to or to about 15%, from or from about 5% to or to about 10%, from or from about 10% to or to about 50%, from or from about 10% to or to about 45%, from or from about 10% to or to about 40%, from or from about 10% to or to about 35%, from or from about 10% to or to about 30%, from or from about 10% to or to about 25%, from or from about 10% to or to about 20%, from or from about 10% to or to about 15%, from or from about 15% to or to about 50%, from or from about 15% to or to about 45%, from or from about 15% to or to about 40%, from or from about 15% to or to about 35%, from or from about 15% to or to about 30%, from or from about 15% to or to about 25%, from or from about 15% to or to about 20%, from or from about 20% to or to about 50%, from or from about 20% to or to about 45%, from or from about 20% to or to about 40%, from or from about 20% to or to about 35%, from or from about 20% to or to about 30%, from or from about 20% to or to about 25%, from or from about 25% to or to about 50%, from or from about 25% to or to about 45%, from or from about 25% to or to about 40%, from or from about 25% to or to about 35%, from or from about 25% to or to about 30%, from or from about 30% to or to about 50%, from or from about 30% to or to about 45%, from or from about 30% to or to about 40%, from or from about 30% to or to about 35%, from or from about 35% to or to about 50%, from or from about 35% to or to about 45%, from or from about 35% to or to about 40%, from or from about 40% to or to about 50%, from or from about 40% to or to about 45%, or from or from about 45% to or to about 50% w/w dextran, e.g., Dextran 40. In some embodiments, the dextran solution comprises 5%, 10%, 15%, 20%, 25%, 30%, 35%, 40%, 45%, or 50% w/w dextran, e.g., Dextran 40. In some embodiments, the dextran solution comprises about 5%, about 10%, about 15%, about 20%, about 25%, about 30%, about 35%, about 40%, about 45%, or about 50% w/w dextran, e.g., Dextran 40.

In some embodiments, the dextran solution comprises from or from about 25 g/L to or to about 200 g/L dextran, e.g., Dextran 40. In some embodiments, the dextran solution comprises from or from about 35 to or to about 200, from or from about 25 to or to about 175, from or from about 25 to or to about 150, from or from about 25 to or to about 125, from or from about 25 to or to about 100, from or from about 25 to or to about 75, from or from about 25 to or to about 50, from or from about 50 to or to about 200, from or from about 50 to or to about 175, from or from about 50 to or to about 150, from or from about 50 to or to about 125, from or from about 50 to or to about 100, from or from about 50 to or to about 75, from or from about 75 to or to about 200, from or from about 75 to or to about 175, from or from about 75 to or to about 150, from or from about 75 to or to about 125, from or from about 75 to or to about 100, from or from about 100 to or to about 200, from or from about 100 to or to about 175, from or from about 100 to or to about 150, from or from about 100 to or to about 125, from or from about 125 to or to about 200, from or from about 125 to or to about 175, from or from about 125 to or to about 150, from or from about 150 to or to about 200, from or from about 150 to or to about 175, or from or from about 175 to or to about 200 g/L dextran e.g., Dextran 40. In some embodiments, the dextran solution comprises 25, 50, 75, 100, 125, 150, 175, or 200 g/L dextran, e.g., Dextran 40. In some embodiments, the dextran solution comprises 100 g/L dextran, e.g., Dextran 40. In some embodiments, the dextran solution comprises about 25, about 50, about 75, about 100, about 125, about 150, about 175, or about 200 g/L dextran, e.g., Dextran 40. In some embodiments, the dextran solution comprises about 100 g/L dextran, e.g., Dextran 40.

In some embodiments, the dextran solution further comprises glucose (also referred to as dextrose). In some embodiments, the dextran solution comprises from or from about 10 g/L to or to about 100 g/L glucose. In some embodiments, the dextran solution comprises from or from about 10 to or to about 100, from or from about 10 to or to about 90, from or from about 10 to or to about 80, from or from about 10 to or to about 70, from or from about 10 to or to about 60, from or from about 10 to or to about 50, from or from about 10 to or to about 40, from or from about 10 to or to about 30, from or from about 10 to or to about 20, from or from about 20 to or to about 100, from or from about 20 to or to about 90, from or from about 20 to or to about 80, from or from about 20 to or to about 70, from or from about 20 to or to about 60, from or from about 20 to or to about 50, from or from about 20 to or to about 40, from or from about 20 to or to about 30, from or from about 30 to or to about 100, from or from about 30 to or to about 90, from or from about 30 to or to about 80, from or from about 30 to or to about 70, from or from about 30 to or to about 60, from or from about 30 to or to about 50, from or from about 30 to or to about 40, from or from about 40 to or to about 100, from or from about 40 to or to about 90, from or from about 40 to or to about 80, from or from about 40 to or to about 70, from or from about 40 to or to about 60, from or from about 40 to or to about 50, from or from about 50 to or to about 100, from or from about 50 to or to about 90, from or from about 50 to or to about 80, from or from about 50 to or to about 70, from or from about 50 to or to about 60, from or from about 60 to or to about 100, from or from about 60 to or to about 90, from or from about 60 to or to about 80, from or from about 60 to or to about 70, from or from about 70 to or to about 100, from or from about 70 to or to about 90, from or from about 70 to or to about 80, from or from about 80 to or to about 90, from or from about 80 to or to about 100, from or from about 80 to or to about 90, or from or from about 90 to or to about 100 g/L glucose. In some embodiments, the dextran solution comprises 10, 20, 30, 40, 50, 60, 70, 80, 90, or 100 g/L glucose. In some embodiments, the dextran solution comprises 50 g/L glucose. In some embodiments, the dextran solution comprises about 10, about 20, about 30, about 40, about 50, about 60, about 70, about 80, about 90, or about 100 g/L glucose. In some embodiments, the dextran solution comprises 50 g/L glucose.

In some embodiments, the dextran solution consists of dextran, e.g., Dextran 40, and glucose in water.

In some embodiments, the cryopreservation composition comprises from or from about 10% v/v to or to about 50% v/v of a dextran solution described herein. In some embodiments, the cryopreservation composition comprises from or from about 10% to 50%, from or from about 10% to or to about 45%, from or from about 10% to or to about 40%, from or from about 10% to or to about 35%, from or from about 10% to or to about 30%, from or from about 10% to or to about 25%, from or from about 10% to or to about 20%, from or from about 10% to or to about 15%, from or from about 15% to or to about 50%, from or from about 15% to or to about 45%, from or from about 15% to or to about 40%, from or from about 15% to or to about 35%, from or from about 15% to or to about 30%, from or from about 15% to or to about 25%, from or from about 15% to or to about 20%, from or from about 20% to or to about 50%, from or from about 20% to or to about 45%, from or from about 20% to or to about 40%, from or from about 20% to or to about 35%, from or from about 20% to or to about 30%, from or from about 20% to or to about 25%, from or from about 25% to or to about 50%, from or from about 25% to or to about 45%, from or from about 25% to or to about 40%, from or from about 25% to or to about 35%, from or from about 25% to or to about 30%, from or from about 30% to or to about 50%, from or from about 30% to or to about 45%, from or from about 30% to or to about 40%, from or from about 30% to or to about 35%, from or from about 35% to or to about 50%, from or from about 35% to or to about 45%, from or from about 35% to or to about 40%, from or from about 40% to or to about 50%, from or from about 40% to or to about 45%, or from or from about 45% to or to about 50% v/v of a dextran solution, e.g., a dextran solution described herein. In some embodiments, the cryopreservation composition comprises 10%, 15%, 20%, 25%, 30%, 35%, 40%, 45%, or 50% v/v of a dextran solution, e.g., a dextran solution described herein. In some embodiments, the cryopreservation composition comprises about 10%, about 15%, about 20%, about 25%, about 30%, about 35%, about 40%, about 45%, or about 50% v/v of a dextran solution, e.g., a dextran solution described herein.

In some embodiments, the cryopreservation composition comprises from or from about 10 to or to about 50 g/L dextran, e.g., Dextran 40. In some embodiments, the cryopreservation composition comprises from or from about 10 to or to about 50, from or from about 10 to or to about 45, from or from about 10 to or to about 40, from or from about 10 to or to about 35, from or from about 10 to or to about 30, from or from about 10 to or to about 25, from or from about 10 to or to about 20, from or from about 10 to or to about 15, from or from about 15 to or to about 50, from or from about 15 to or to about 45, from or from about 15 to or to about 40, from or from about 15 to or to about 35, from or from about 15 to or to about 30, from or from about 15 to or to about 25, from or from about 15 to or to about 20, from or from about 20 to or to about 50, from or from about 20 to or to about 45, from or from about 20 to or to about 40, from or from about 20 to or to about 30, from or from about 20 to or to about 25, from or from about 25 to or to about 50, from or from about 25 to or to about 45, from or from about 25 to or to about 40, from or from about 25 to or to about 35, from or from about 25 to or to about 30, from or from about 30 to or to about 50, from or from about 30 to or to about 45, from or from about 30 to or to about 40, from or from about 30 to or to about 35, from or from about 35 to or to about 50, from or from about 35 to or to about 45, from or from about 35 to or to about 40, from or from about 40 to or to about 50, from or from about 40 to or to about 45, or from or from about 45 to or to about 50 g/L dextran, e.g., Dextran 40. In some embodiments, the cryopreservation composition comprises 10, 15, 20, 25, 30, 30, 35, 40, 45, or 50 g/L dextran, e.g., Dextran 40. In some embodiments, the cryopreservation composition comprises about 10, about 15, about 20, about 25, about 30, about 30, about 35, about 40, about 45, or about 50 g/L dextran, e.g., Dextran 40.

### 3. Glucose

In some embodiments, the cryopreservation composition comprises glucose.

In some embodiments, as described above, the cryopreservation composition comprises a Dextran solution comprising glucose.

In some embodiments, the cryopreservation composition comprises a Dextran solution that does not comprise glucose. In some embodiments, e.g., when the Dextran solution does not comprise glucose, glucose is added separately to the cryopreservation composition.

In some embodiments, the cryopreservation composition comprises from or from about 5 to or to about 25 g/L glucose. In some embodiments, the cryopreservation composition comprises from or from about 5 to or to about 25, from or from about 5 to or to about 20, from or from about 5 to or to about 15, from or from about 5 to or to about 10, from or from about 10 to or to about 25, from or from about 10 to or to about 20, from or from about 10 to or to about 15, from or from about 15 to or to about 25, from or from about 15 to or to about 20, or from or from about 20 to or to about 25 g/L glucose. In some embodiments, the cryopreservation composition comprises 5, 7.5, 10, 12.5, 15, 17.5, 20, 22.5, or 25 g/L glucose. In some embodiments, the cryopreservation composition comprises 12.5 g/L glucose. In some embodiments, the cryopreservation composition comprises about 5, about 7.5, about 10, about 12.5, about 15, about 17.5, about 20, about 22.5, or about 25 g/L glucose. In some embodiments, the cryopreservation composition comprises about 12.5 g/L glucose.

In some embodiments, the cryopreservation composition comprises less than 2.75% w/v glucose. In some embodiments, the cryopreservation composition comprises less than 27.5 g/L glucose. In some embodiments, the cryopreservation composition comprises less than 2% w/v glucose. In some embodiments, the cryopreservation composition comprises less than 1.5% w/v glucose. In some embodiments, the cryopreservation composition comprises about 1.25% w/v or less glucose.

### 4. Dimethyl Sulfoxide

In some embodiments, the cryopreservation composition comprises dimethyl sulfoxide (DMSO, also referred to as methyl sulfoxide and methylsulfinylmethane).

In some embodiments, the DMSO is provided as a solution, also referred to herein as a DMSO solution. Thus, in some embodiments, the cryopreservation composition comprises a DMSO solution.

In some embodiments, the DMSO solution is suitable for intravenous use.

In some embodiments, the DMSO solution comprises 1.1 g/mL DMSO. In some embodiments, the DMSO solution comprises about 1.1 g/mL DMSO.

In some embodiments, the cryopreservation composition comprises from or from about 1% to or to about 10% v/v of the DMSO solution. In some embodiments, the cryopreservation composition comprises from or from about 1% to or to about 10%, from or from about 1% to or to about 9%, from or from about 1% to or to about 8%, from or from about 1% to or to about 7%, from or from about 1% to or to about 6%, from or from about 1% to or to about 5%, from or from about 1% to or to about 4%, from or from about 1% to or to about 3%, from or from about 1% to or to about 2%, from or from about 2% to or to about 10%, from or from about 2% to or to about 9%, from or from about 8%, from or from about 2% to or to about 7%, from or from about 2% to or to about 6%, from or from about 2% to or to about 5%, from or from about 2% to or to about 4%, from or from about 2% to or to about 3%, from or from about 3% to or to about 10%, from or from about 3% to or to about 9%, from or from about 3% to or to about 8%, from or from about 3% to or to about 7%, from or from about 3% to or to about 6%, from or from about 3% to or to about 5%, from or from about 3% to or to about 4%, from or from about 4% to or to about 10%, from or from about 4% to or to about 9%, from or from about 4% to or to about 8%, from or from about 4% to or to about 7%, from or from about 4% to or to about 6%, from or from about 4% to or to about 5%, from or from about 5% to or to about 10%, from or from about 5% to or to about 9%, from or from about 5% to or to about 8%, from or from about 5% to or to about 7%, from or from about 5% to or to about 6%, from or from about 6% to or to about 10%, from or from about 6% to or to about 9%, from or from about 6% to or to about 8%, from or from about 6% to or to about 7%, from or from about 7% to or to about 10%, from or from about 7% to or to about 9%, from or from about 7% to or to about 8%, from or from about 8% to or to about 10%, from or from about 8% to or to about 9%, or from or from about 9% to or to about 10% v/v of the DMSO solution. In some embodiments, the cryopreservation composition comprises 1%, 2%, 3%, 4%, 5%, 6%, 7%, 8%, 9%, or 10% v/v of the DMSO solution. In some embodiments, the cryopreservation composition comprises 5% of the DMSO solution. In some embodiments, the cryopreservation composition comprises about 1%, about 2%, about 3%, about 4%, about 5%, about 6%, about 7%, about 8%, about 9%, or about 10% v/v of the DMSO solution. In some embodiments, the cryopreservation composition comprises about 5% of the DMSO solution.

In some embodiments, the cryopreservation composition comprises from or from about 11 to or to about 110 g/L DMSO. In some embodiments, from or from about the cryopreservation composition comprises from or from about 11 to or to about 110, from or from about 11 to or to about 99, from or from about 11 to or to about 88, from or from about 11 to or to about 77, from or from about 11 to or to about 66, from or from about 11 to or to about 55, from or from about 11 to or to about 44, from or from about 11 to or to about 33, from or from about 11 to or to about 22, from or from about 22 to or to about 110, from or from about 22 to or to about 99, from or from about 22 to or to about 88, from or from about 22 to or to about 77, from or from about 22 to or to about 77, from or from about 22 to or to about 66, from or from about 22 to or to about 55, from or from about 22 to or to about 44, from or from about 22 to or to about 33, from or from about 33 to or to about 110, from or from about 33 to or to about 99, from or from about 33 to or to about 88, from or from about 33 to or to about 77, from or from about 33 to or to about 66, from or from about 33 to or to about 55, from or from about 33 to or to about 44, from or from about 44 to or to about 110, from or from about 44 to or to about 99, from or from about 44 to or to about 88, from or from about 44 to or to about 77, from or from about 44 to or to about 66, from or from about 44 to or to about 55, from or from about 55 to or to about 110, from or from about 55 to or to about 99, from or from about 55 to or to about 88, from or from about 55 to or to about 77, from or from about 55 to or to about 66, from or from about 66 to or to about 110, from or from about 66 to or to about 99, from or from about 66 to or to about 88, from or from about 66 to or to about 77, from or from about 77 to or to about 119, from or from about 77 to or to about 88, from or from about 88 to or to about 110, from or from about 88 to or to about 99, or from or from about 99 to or to about 110 g/L DMSO. In some embodiments, the cryopreservation composition comprises 11, 22, 33, 44, 55, 66, 77, 88, 99, or 110 g/L DMSO. In some embodiments, the cryopreservation composition comprises 55 g/L DMSO. In some embodiments, the cryopreservation composition comprises about 11, about 22, about 33, about 44, about 55, about 66, about 77, about 88, about 99, or about 110 g/L DMSO. In some embodiments, the cryopreservation composition comprises about 55 g/L DMSO.

### 5. Buffers

In some embodiments, the cryopreservation composition comprises a buffer solution, e.g., a buffer solution suitable for intravenous administration.

Buffer solutions include, but are not limited to, phosphate buffered saline (PBS), Ringer's Solution, Tyrode's buffer, Hank's balanced salt solution, Earle's Balanced Salt Solution, saline, and Tris.

In some embodiments, the buffer solution is phosphate buffered saline (PBS).

### 6. Exemplary Cryopreservation Compositions

In some embodiments, the cryopreservation composition comprises or consists of: 1) albumin, e.g., human albumin, 2) dextran, e.g., Dextran 40, 3) DMSO, and 4) a buffer solution. In some embodiments, the cryopreservation composition further comprises glucose. In some embodiments, the cryopreservation composition consists of 1) albumin, e.g., human albumin, 2) dextran, e.g., Dextran 40, 3) glucose, 4) DMSO, and 5) a buffer solution.

In some embodiments, the cryopreservation composition comprises: 1) an albumin solution described herein, 2) a dextran solution described herein, 3) a DMSO solution described herein, and 4) a buffer solution.

In some embodiments, the cryopreservation composition consists of: 1) an albumin solution described herein, 2) a dextran solution described herein, 3) a DMSO solution described herein, and 4) a buffer solution.

In some embodiments, the cryopreservation composition does not comprise a cell culture medium.

In one embodiment, the cryopreservation composition comprises or comprises about 40 mg/mL human albumin, 25 mg/mL Dextran 40, 12.5 mg/mL glucose, and 55 mg/mL DMSO.

In one embodiment, the cryopreservation composition comprises or comprises about or consists of or consists of about 40 mg/mL human albumin, 25 mg/mL Dextran 40, 12.5 mg/mL glucose, 55 mg/mL DMSO, and 0.5 mL/mL 100% phosphate buffered saline (PBS) in water.

In one embodiment, the cryopreservation composition comprises or comprises about 32 mg/mL human albumin, 25 mg/mL Dextran 40, 12.5 mg/mL glucose, and 55 mg/mL DMSO.

In one embodiment, the cryopreservation composition comprises or comprises about or consists of or consists of about 32 mg/mL human albumin, 25 mg/mL Dextran 40, 12.5 mg/mL glucose, 55 mg/mL DMSO, and 0.54 mL/mL 100% phosphate buffered saline (PBS) in water.

Exemplary Cryopreservation Compositions are shown in Table 3.

TABLE 3
Exemplary Cryopreservation Compositions

| | | | Exemplary |
|---|---|---|---|
| | Concentration | Exemplary | Range v/v % in |
| Excipient | Range of | Solution | Cryopreservation |
| Solution | Solution | Concentration | Composition |
| Albumin | 40-200 g/L albumin | 200 g/L albumin | 10%-50% |
| Solution | in water | | |
| Dextran 40 | 25-200 g/L Dextran | 100 g/L Dextran | 10%-50% |
| Solution | 40; and | 40; | |
| | 0-100 g/L glucose | 50 g/L glucose | |
| | in water | | |
| DMSO | 11-110 g/L DMSO | 1,100 g/L DMSO | 1%-10% |
| | in water | | |
| Buffer | to volume | to volume | to volume |

TABLE 4
Exemplary Cryopreservation Composition #1

| | | Exemplary | Final | |
|---|---|---|---|---|
| | | v/v % in | Concentration in | |
| Excipient | Solution | Cryopreservation | Cryopreservation | |
| Solution | Composition | Composition #1 | Composition #1 | |
| Albumin | 200 g/L albumin | 20% | 40 mg/mL albumin | |
| Solution | in water | | | |
| Dextran 40 | 100 g/L Dextran | 25% | 25 mg/mL Dextran | |
| Solution | 40; and | | 40; | |
| | 50 g/L glucose | | 12.5 mg/mL glucose | |
| | in water | | | |
| DMSO | 100% DMSO | 5% | 55 | mg/mL |
| | (1,100 g/L) | | | |
| Buffer | 100% Phosphate | 50% | 0.5 | mL/mL |
| | Buffered | | | |
| | Saline (PBS) | | | |

TABLE 5
Exemplary Cryopreservation Composition #2

| | | Exemplary | Final | |
|---|---|---|---|---|
| | | v/v % in | Concentration in | |
| Excipient | Solution | Cryopreservation | Cryopreservation | |
| Solution | Composition | Composition #2 | Composition #2 | |
| Albumin | 200 g/L albumin | 16% | 32 mg/mL albumin | |
| Solution | in water | | | |
| Dextran 40 | 100 g/L Dextran | 25% | 25 mg/mL Dextran | |
| Solution | 40; and | | 40; | |
| | 50 g/L glucose | | 12.5 mg/mL glucose | |
| | in water | | | |
| DMSO | 100% DMSO | 5% | 55 | mg/mL |
| | (1,100 g/L) | | | |
| Buffer | 100% Phosphate | 54% | 0.54 | mL/mL |
| | Buffered | | | |
| | Saline (PBS) | | | |

### B. Methods of Cryopreserving

The cryopreservation compositions described herein can be used for cryopreserving cell(s), e.g., therapeutic cells, e.g., natural killer (NK) cell(s), e.g., the NK cell(s) described herein.

In some embodiments, the cell(s) are an animal cell(s). In some embodiments, the cell(s) are human cell(s).

In some embodiments, the cell(s) are immune cell(s). In some embodiments, the immune cell(s) are selected from basophils, eosinophils, neutrophils, mast cells, monocytes, macrophages, neutrophils, dendritic cells, natural killer cells, B cells, T cells, and combinations thereof.

In some embodiments, the immune cell(s) are natural killer (NK) cells. In some embodiments, the natural killer cell(s) are expanded and stimulated by a method described herein.

In some embodiments, cryopreserving the cell(s) comprises: mixing the cell(s) with a cryopreservation composition or components thereof described herein to produce a composition, e.g., a pharmaceutical composition; and freezing the mixture.

In some embodiments, cryopreserving the cell(s) comprises: mixing a composition comprising the cell(s) with a cryopreservation composition or components thereof described herein to produce a composition, e.g., a pharmaceutical composition; and freezing the mixture. In some embodiments, the composition comprising the cell(s) comprises: the cell(s) and a buffer. Suitable buffers are described herein.

In some embodiments, cryopreserving the cell(s) comprises: mixing a composition comprising the cell(s) and a buffer, e.g., PBS, with a composition comprising albumin, Dextran, and DMSO, e.g., as described herein; and freezing the mixture.

In some embodiments, cryopreserving the cell(s) comprises: mixing a composition comprising the cell(s) and a buffer, e.g., PBS 1:1 with a composition comprising 40 mg/mL albumin, e.g., human albumin, 25 mg/mL Dextran, e.g., Dextran 40, 12.5 mg/ml glucose and 55 mg/mL DMSO.

In some embodiments, the composition comprising the cell(s) and the buffer, e.g., PBS, comprises from or from about 2×10⁷ to or to about 2×10⁹ cells/mL. In some embodiments, the composition comprising the cell(s) and the buffer, e.g., PBS, comprises 2×10⁸ cells/mL. In some embodiments, the composition comprising the cell(s) and the buffer, e.g., PBS, comprising about 2×10⁸ cells/mL.

In some embodiments, cryopreserving the cell(s) comprising mixing: the cell(s), a buffer, e.g., PBS, albumin, e.g., human albumin, Dextran, e.g., Dextran 40, and DMSO; and freezing the mixture.

In some embodiments, the mixture comprises from or from about 1×10⁷ to or to about 1×10⁹ cells/mL. In some embodiments, the mixture comprises 1×10⁸ cells/mL. In some embodiments, the mixture comprises about 1×10⁸ cells/mL.

Suitable ranges for albumin, Dextran, and DMSO are set forth above.

In some embodiments, the composition is frozen at or below −135° C.

In some embodiments, the composition is frozen at a controlled rate.

In some embodiments, the composition further comprises a multispecific engager, e.g., a multispecific engager described herein.

### III. MULTISPECIFIC ENGAGERS

As used herein, the term “multispecific engager” refers to an antibody construct which is “at least bispecific”, i.e., it comprises at least a first binding domain and a second binding domain, wherein the first binding domain binds to one antigen or target (here: NK cell receptor, e.g. CD16a), and the second binding domain binds to another antigen or target (here: the target cell surface antigen CD30). Accordingly, antibody constructs as defined in the context of the present disclosure comprise specificities for at least two different antigens or targets. For example, the first domain preferably binds to an extracellular epitope of an NK cell receptor of one or more of the species selected from human, Macaca species and rodent species.

Multispecific antibody constructs include, for example, bispecific and trispecific antibody constructs, or constructs having more than three (e.g. four, five . . . ) specificities. Examples of multispecific antibody constructs are provided, for example, in WO 2006/125668, WO 2015/158636, WO 2017/064221, WO 2019/175368, WO 2019/198051, WO 2020/043670, WO 2021/130383, and Ellwanger et a. (MAbs. 2019 July: 11 (5): 899-918).

In some embodiments, the multispecific engager is a bispecific antibody or antigen binding fragment thereof comprising a first binding domain that specifically binds to CD16 (FcγRIII) and a second binding domain that specifically binds to CD30.

In some embodiments, the multispecific engager is a bispecific engager. In some embodiments, the bispecific engager comprises a CD16 binding domain and a CD30 binding domain.

In some embodiments, the CD16 binding domain comprises a light chain variable domain (VL_CD16A) comprising a light chain complementarity determining region 1 (CDRL1) comprising SEQ ID NO: 9, a light chain complementarity determining region 2 (CDRL2) comprising SEQ ID NO: 10; a light chain complementarity determining region 3 (CDRL3) comprising SEQ ID NO: 11; and a heavy chain variable domain (VH_CD16A) comprising a heavy chain complementarity determining region 1 (CDRH1 comprising SEQ ID NO: 6: a heavy chain complementarity determining region 2 (CDRH2) comprising SEQ ID NO: 7; and a heavy chain complementarity determining region 3 (CDRH3) comprising SEQ ID NO: 8.

In some embodiments, the CD16 binding domain comprises a light chain variable (VL) region comprising or consisting of an amino acid sequence having or having at least 85%, 86%, 87%, 88%, 89%, 90%, 91%, 92%, 93%, 94%, 95%, 96%, 97%, 98%, or 99% identity to SEQ ID NO: 20 and a heavy chain variable (VH) region comprising or consisting of an amino acid sequence having or having at least 85%, 86%, 87%, 88%, 89%, 90%, 91%, 92%, 93%, 94%, 95%, 96%, 97%, 98%, or 99% identity to SEQ ID NO: 19.

In some embodiments, the CD16 binding domain comprises a light chain variable (VL) region comprising or consisting of SEQ ID NO: 20 and a heavy chain variable (VH) region comprising or consisting of SEQ ID NO: 19.

In some embodiments, the CD30 binding domain comprises: a light chain variable domain (VL_CD30) comprising a light chain complementarity determining region 1 (CDRL1) comprising SEQ ID NO: 15, a light chain complementarity determining region 2 (CDRL2) comprising SEQ ID NO: 16: a light chain complementarity determining region 3 (CDRL3) comprising SEQ ID NO: 17; and a heavy chain variable domain (VH_CD30) comprising a heavy chain complementarity determining region 1 (CDRH1) comprising SEQ ID NO: 12: a heavy chain complementarity determining region 2 (CDRH2) comprising SEQ ID NO: 13; and a heavy chain complementarity determining region 3 (CDRH3) comprising SEQ ID NO: 14.

In some embodiments, the CD30 binding domain comprises a light chain variable (VL) region comprising or consisting of an amino acid sequence having or having at least 85%, 86%, 87%, 88%, 89%, 90%, 91%, 92%, 93%, 94%, 95%, 96%, 97%, 98%, or 99% identity to SEQ ID NO: 22 and a heavy chain variable (VH) region comprising or consisting of an amino acid sequence having or having at least 85%, 86%, 87%, 88%, 89%, 90%, 91%, 92%, 93%, 94%, 95%, 96%, 97%, 98%, or 99% identity to SEQ ID NO: 21.

In some embodiments, the CD30 binding domain comprises: a light chain variable (VL) region comprising SEQ ID NO: 22 and a heavy chain variable (VH) region comprising SEQ ID NO: 21.

In some embodiments, the multispecific engager is a tetravalent homodimer. In some embodiments, the tetravalent homodimer comprises a first and second polypeptide monomer, each comprising or consisting of, from the N-terminus to the C-terminus: a CD16A heavy chain variable domain (VH_CD16A)—a first linker (L1)—a CD30 light chain variable domain (VL_CD30)—a second linker (L2)—a CD30 heavy chain variable domain (VH_CD30)—a third linker (L3)—and a CD16 light chain variable domain (VL_CD16). In some embodiments, the first and second polypeptides dimerize from head to tail through non-covalent interactions of the domains in the Ig heavy (VH) and light (VL) variable chains.

In some embodiments, the multispecific engager is a tetravalent homodimer. In some embodiments, the tetravalent homodimer comprises a first and second polypeptide monomer, each comprising or consisting of, from the N-terminus to the C-terminus: a CD30 heavy chain variable domain (VH_CD30)—a first linker (L1)—a CD16A light chain variable domain (VL_CD16A)—a second linker (L2)—a CD16A heavy chain variable domain (VH_CD16A)—a third linker (L3)—and a CD30 light chain variable domain (VL_CD30). In some embodiments, the first and second polypeptides dimerize from head to tail through non-covalent interactions of the domains in the Ig heavy (VH) and light (VL) variable chains.

In some embodiments, VH_CD16 comprises a heavy chain complementarity determining region 1 (CDRH1 comprising SEQ ID NO: 6; a heavy chain complementarity determining region 2 (CDRH2) comprising SEQ ID NO: 7; and a heavy chain complementarity determining region 3 (CDRH3) comprising SEQ ID NO: 8. In some embodiments, VH_CD16 comprises or consists of SEQ ID NO: 19.

In some embodiments, VL_CD16 comprises a light chain complementarity determining region 1 (CDRL1) comprising SEQ ID NO: 9, a light chain complementarity determining region 2 (CDRL2) comprising SEQ ID NO: 10; and a light chain complementarity determining region 3 (CDRL3) comprising SEQ ID NO: 11. In some embodiments, VL_CD16A comprises or consists of SEQ ID NO: 20.

In some embodiments, VH_CD30 comprises a heavy chain complementarity determining region 1 (CDRH1 comprising SEQ ID NO: 12: a heavy chain complementarity determining region 2 (CDRH2) comprising SEQ ID NO: 13; and a heavy chain complementarity determining region 3 (CDRH3) comprising SEQ ID NO: 14. In some embodiments, VH_CD30 comprises or consists of SEQ ID NO: 21.

In some embodiments, VL_CD30 comprises a light chain complementarity determining region 1 (CDRL1) comprising SEQ ID NO: 15: a light chain complementarity determining region 2 (CDRL2) comprising SEQ ID NO: 16; and a light chain complementarity determining region 3 (CDRL3) comprising SEQ ID NO: 17. In some embodiments, VL_CD16A comprises or consists of SEQ ID NO: 22.

In some embodiments, L1 comprises or consists of SEQ ID NO: 26.

In some embodiments, L2 comprises or consists of SEQ ID NO: 27.

In some embodiments, L3 comprises or consists of SEQ ID NO: 28.

In some embodiments, the first and second polymeric monomers each comprise or consists of SEQ ID NO: 18.

In some embodiments, the multispecific engager comprises a tag, e.g., a histidine tag, e.g., a hexa-histidine tag (SEQ ID NO: 25).

In some embodiments, the multispecific engager is AFM13. See. e.g., Wu et al., “AFF13: a first-in-class tetravalent bispecific anti-CD30/CD16A antibody for NK cell-mediated immunotherapy,” J. Hemat & Oncol 8, 96 (2015), which is hereby incorporated by reference in its entirety: see also Reusch et al., “A Novel Tetravalent Bispecific TandAb (CD30/CD16A) efficiently recruits NK cells for the lysis of CD30+tumor cells,” mAbs 6 (3): 727-38 (2014), which is hereby incorporated by reference in its entirety, Rothe et al., “A Phase 1 Study fo the Bispecific Anti-CD30/CD16A Antibody Construct AFM13 in Patients with Relapsed or Refractory Hodgkin Lymphoma,” Blood 125 (26): 4024-31 (2015), which is hereby incorporated by reference in its entirety.

As used herein, the term “NK cell receptor” defines proteins and protein complexes on the surface of NK cells. Thus, the term defines cell surface molecules, which are characteristic to NK cells, but are not necessary exclusively expressed on the surface of NK cells but also on other cells such as macrophages or T cells. Examples for NK cell receptors comprise, but are not limited to FcγRIII (CD16a, CD16b), NKp46 and NKG2D.

As used herein, “CD16a” refers to the activating receptor CD16a, also known as FcγRIIIA, expressed on the cell surface of NK cells. CD16a is an activating receptor triggering the cytotoxic activity of NK cells. The affinity of antibodies for CD16a directly correlates with their ability to trigger NK cell activation, thus higher affinity towards CD16a reduces the antibody dose required for activation. The antigen-binding site of the antigen-binding protein binds to CD16a, but not to CD16b. For example, an antigen-binding site comprising heavy (VH) and light (VL) chain variable domains binding to CD16a, but not binding to CD16B, may be provided by an antigen binding site which specifically binds to an epitope of CD16a which comprises amino acid residues of the C-terminal sequence SFFPPGYQ (SEQ ID NO: 23) and/or residues G130 and/or Y141 of CD16a (SEQ ID NO: 24) which are not present in CD16b.

As used herein, “CD16b” refers to receptor CD16b, also known as FcγRIIIB, expressed on neutrophils and eosinophils. The receptor is glycosylphosphatidyl inositol (GPI) anchored and is understood to not trigger any kind of cytotoxic activity of CD16b positives immune cells.

As used herein, the term “target cell surface antigen” refers to an antigenic structure expressed by a cell and which is present at the cell surface such that it is accessible for an antibody construct as described herein. In some cases, the “target cell surface antigen” to which the multispecific antibody constructs described herein binds to is CD30. CD30 also known as TNFRSF8, is a cell membrane protein of the tumor necrosis factor receptor family and tumor marker.

Given that the antibody constructs as defined in the context of the invention are (at least) bispecific, they do not occur naturally and they are markedly different from naturally occurring products. A “multispecific” antibody construct or immunoglobulin is hence an artificial hybrid antibody or immunoglobulin having at least two distinct binding sides with different specificities. Multispecific antibody constructs can be produced by a variety of methods including fusion of hybridomas or linking of Fab′ fragments. See, e.g., Songsivilai & Lachmann, Clin. Exp. Immunol. 79:315-321 (1990).

The at least two binding domains and the variable domains (VH/VL) of the antibody construct of the present disclosure may or may not comprise peptide linkers (spacer or connector peptides). In some embodiments, the term “peptide linker” comprises an amino acid sequence by which the amino acid sequences of one (variable and/or binding) domain and another (variable and/or binding) domain of the antibody construct defined herein are linked with each other. The peptide linkers can also be used to fuse the third domain to the other domains or an Fc part of the antibody construct defined herein. Preferably, such peptide linker does not comprise any polymerization activity.

As used herein, the term “binding domain” characterizes a domain which (specifically) binds to/interacts with/recognizes a given target epitope or a given target side on the target molecules (antigens), e.g. a NK cell receptor antigen, e.g. CD16, and the target cell surface antigen CD30, respectively. The structure and function of the first binding domain (recognizing e.g. CD16), and preferably also the structure and/or function of the second binding domain (recognizing the target cell surface antigen), is/are based on the structure and/or function of an antibody, e.g. of a full-length or whole immunoglobulin molecule and/or is/are drawn from the variable heavy chain (VH) and/or variable light chain (VL) domains of an antibody or fragment thereof. Preferably the first binding domain is characterized by the presence of three light chain CDRs (i.e. CDR1, CDR2 and CDF3 of the VL region) and/or three heavy chain CDRs (i.e. CDR1, CDR2 and CDR3 of the VH region). The second binding domain preferably also comprises the minimum structural requirements of an antibody which allow for the target binding. More preferably, the second binding domain comprises at least three light chain CDRs (i.e. CDR1, CDR2 and CDR3 of the VL region) and/or three heavy chain CDRs (i.e. CDRl, CDR2 and CDR3 of the VH region). In some cases, the first and/or second binding domain is produced by or obtainable by phage-display or library screening methods rather than by grafting CDR sequences from a pre-existing (monoclonal) antibody into a scaffold.

In some embodiments, binding domains are in the form of one or more polypeptides. Such polypeptides may include proteinaceous parts and non-proteinaceous parts (e.g. chemical linkers or chemical cross-linking agents such as glutaraldehyde). Proteins (including fragments thereof, preferably biologically active fragments, and peptides, usually having less than 30 amino acids) comprise two or more amino acids coupled to each other via a covalent peptide bond (resulting in a chain of amino acids).

As used herein, the term “polypeptide” describes a group of molecules, which usually consist of more than 30 amino acids. Polypeptides may further form multimers such as dimers, trimers and higher oligomers. i.e., consisting of more than one polypeptide molecule. Polypeptide molecules forming such dimers, trimers etc. may be identical or non-identical. The corresponding higher order structures of such multimers are, consequently, termed homo- or heterodimers, homo- or heterotrimers etc. An example for a heteromultimer is an antibody molecule, which, in its naturally occurring form, consists of two identical light polypeptide chains and two identical heavy polypeptide chains. The terms “peptide”, “polypeptide” and “protein” also refer to naturally modified peptides/polypeptides/proteins wherein the modification is affected e.g, by post-translational modifications like glycosylation, acetylation, phosphorylation and the like. A “peptide”, “polypeptide” or “protein” when referred to herein may also be chemically modified such as pegylated. Such modifications are well known in the art and described herein below.

Preferably the binding domain which binds to the NK cell receptor antigen, e.g. CD16 and/or the binding domain which binds to the target cell surface antigen CD30 is/are human, humanized or murine derived chimeric binding domains. Antibodies and antibody constructs comprising at least one human binding domain avoid some of the problems associated with antibodies or antibody constructs that possess non-human such as rodent (e.g. murine, rat, hamster or rabbit) variable and/or constant regions. The presence of such rodent derived proteins can lead to the rapid clearance of the antibodies or antibody constructs or can lead to the generation of an immune response against the antibody or antibody construct by a patient. In order to avoid the use of rodent derived antibodies or antibody constructs, human or fully human antibodies/antibody constructs can be generated through the introduction of human antibody function into a rodent so that the rodent produces fully human antibodies.

In some embodiments, this antigen-binding site for CD16A does not bind to CD16B and binds to the known CD16A allotypes F158 and V158 with similar affinity. Two allelic single nucleotide polymorphisms have been identified in human CD16A altering the amino acid in position 158, which is important for interaction with the hinge region of IgG. The allelic frequencies of the homozygous 158 F/F and the heterozygous 158 V/F alleles are similar within the Caucasian population, ranging between 35 and 52% or 38 and 50%, respectively, whereas the homozygous 158 V/V allele is only found in 10-15% (Lopez-Escamez. J A et al.; BMC Med Genet 2011; 12: 2). Activation of NK-cells by this anti-CD16A domain in all patients due to the similar affinity is therefore advantageous. Further CD16A antigen-binding sites comprising heavy and light variable chain domains that bind to CD16A, but not to CD16B are described in WO 2006/125668.

In some embodiments, the heavy and light chain domains incorporate immunologically active homologues or variants of the CDR or framework sequences described herein. In some embodiments, a CDR variant sequence is modified to change non-critical residues or residues in non-critical regions. Amino acids that are not critical can be identified by known methods, such as affinity maturation, CDR walking mutagenesis, site-directed mutagenesis, crystallization, nuclear magnetic resonance, photoaffinity labeling, or alanine-scanning mutagenesis.

### IV. PHARMACEUTICAL COMPOSITIONS

Provided herein are pharmaceutical compositions comprising the natural killer cells described herein and dosage units of the pharmaceutical compositions described herein.

In some cases, the dosage unit comprises between 100 million and 1.5 billion cells, e.g., 100 million, 200 million, 300 million, 400 million, 500 million, 600 million, 700 million, 800 million, 900 million, 1 billion, 1.1 billion, 1.2 billion, 1.3 billion, 1.4 billion, or 1.5 billion.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration.

In some embodiments, the pharmaceutical composition comprises: a) natural killer cell(s) described herein; and b) a cryopreservation composition.

Suitable cryopreservation compositions are described herein.

In some embodiments, the composition is frozen. In some embodiments, the composition has been frozen for at least three months, e.g., at least six months, at least nine months, at least 12 months, at least 15 months, at least 18 months, at least 24 months, or at least 36 months.

In some embodiments, at least 60%, e.g., at least 70%, at least 80%, at least 90% at least 95%, at least 99%, or 100% of the natural killer cells are viable after being thawed.

In some embodiments, the pharmaceutical composition comprises: a) a cryopreservation composition described herein; and b) therapeutic cell(s).

In some embodiments, the therapeutic cell(s) are animal cell(s). In some embodiments, the therapeutic cell(s) are human cell(s).

In some embodiments, the therapeutic cell(s) are immune cell(s). In some embodiments, the immune cell(s) are selected from basophils, eosinophils, neutrophils, mast cells, monocytes, macrophages, neutrophils, dendritic cells, natural killer cells, B cells, T cells, and combinations thereof.

In some embodiments, the immune cell(s) are natural killer (NK) cells. In some embodiments, the natural killer cell(s) are expanded and stimulated by a method described herein.

In some embodiments, the pharmaceutical composition further comprises: c) a buffer solution. Suitable buffer solutions are described herein, e.g., as for cryopreservation compositions.

In some embodiments, the pharmaceutical composition comprises from or from about 1×10⁷ to or to about 1×10⁹ cells/mL. In some embodiments, the pharmaceutical composition comprises 1×10⁸ cells/mL. In some embodiments, the pharmaceutical composition comprises about 1×10⁸ cells/mL.

In some embodiments, the pharmaceutical composition further comprises a multispecific engager, e.g., a multispecific engager described herein.

Pharmaceutical compositions are typically formulated to be compatible with its intended route of administration. Examples of routes of administration include parenteral, e.g., intravenous, intradermal, subcutaneous, oral (e.g., inhalation), transdermal (topical), transmucosal, and rectal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

### V. METHODS OF TREATMENT

The NK cells described herein find use for treating cancer or other proliferative disorders.

Thus, also provided herein are methods of treating a patient suffering from a disorder, e.g., a disorder associated with a cancer, e.g., a CD30+ cancer|, comprising administering the NK cells, e.g., the NK cells described herein, and a CD30 targeting multispecific engager, e.g., a multispecific engager described herein.

Also provided herein are methods of preventing, reducing and/or inhibiting the recurrence, growth, proliferation, migration and/or metastasis of a cancer cell or population of cancer cells in a subject in need thereof, comprising administering the NK cells, e.g., the NK cells described herein, and a CD30 targeting multispecific engager, e.g., a multispecific engager described herein.

Also provided herein are methods of enhancing, improving, and/or increasing the response to an anticancer therapy in a subject in need thereof, comprising administering the NK cells, e.g., the NK cells described herein, and a CD30 targeting multispecific engager, e.g., a multispecific engager described herein.

Also provided herein are methods for inducing the immune system in a subject in need thereof comprising administering the NK cells, e.g., the NK cells described herein, and a CD30 targeting multispecific engager, e.g., a multispecific engager described herein.

The methods described herein include methods for the treatment of disorders associated with abnormal apoptotic or differentiative processes, e.g., cellular proliferative disorders or cellular differentiative disorders, e.g., cancer, including both solid tumors and hematopoietic cancers. Generally, the methods include administering a therapeutically effective amount of a treatment as described herein, to a subject who is in need of, or who has been determined to be in need of, such treatment. In some embodiments, the methods include administering a therapeutically effective amount of a treatment comprising an NK cells, e.g., NK cells described herein, and a CD30 targeting multispecific engager, e.g., a multispecific engager described herein.

As used herein, the terms “treatment,” “treat,” and “treating” refer to reversing, alleviating, delaying the onset of, or inhibiting the progress of a disorder associated with abnormal apoptotic or differentiative processes. For example, a treatment can result in a reduction in tumor size or growth rate. Administration of a therapeutically effective amount of a compound described herein for the treatment of a condition associated with abnormal apoptotic or differentiative processes will result in a reduction in tumor size or decreased growth rate, a reduction in risk or frequency of reoccurrence, a delay in reoccurrence, a reduction in metastasis, increased survival, and/or decreased morbidity and mortality, among other things. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

As used herein, the terms “inhibition”, as it relates to cancer and/or cancer cell proliferation, refer to the inhibition of the growth, division, maturation or viability of cancer cells, and/or causing the death of cancer cells, individually or in aggregate with other cancer cells, by cytotoxicity, nutrient depletion, or the induction of apoptosis.

As used herein, “delaying” development of a disease or disorder, or one or more symptoms thereof, means to defer, hinder, slow, retard, stabilize and/or postpone development of the disease, disorder, or symptom thereof. This delay can be of varying lengths of time, depending on the history of the disease and/or subject being treated. As is evident to one skilled in the art, a sufficient or significant delay can, in effect, encompass prevention, in that the subject does not develop the disease, disorder, or symptom thereof. For example, a method that “delays” development of cancer is a method that reduces the probability of disease development in a given time frame and/or reduces extent of the disease in a given time frame, when compared to not using the method. Such comparisons may be based on clinical studies, using a statistically significant number of subjects.

As used herein, “prevention” or “preventing” refers to a regimen that protects against the onset of the disease or disorder such that the clinical symptoms of the disease do not develop. Thus, “prevention” relates to administration of a therapy (e.g., administration of a therapeutic substance) to a subject before signs of the disease are detectable in the subject and/or before a certain stage of the disease (e.g., administration of a therapeutic substance to a subject with a cancer that has not yet metastasized). The subject may be an individual at risk of developing the disease or disorder, or at risk of disease progression, e.g., cancer metastasis. Such as an individual who has one or more risk factors known to be associated with development or onset of the disease or disorder. For example, an individual may have mutations associated with the development or progression of a cancer. Further, it is understood that prevention may not result in complete protection against onset of the disease or disorder. In some instances, prevention includes reducing the risk of developing the disease or disorder. The reduction of the risk may not result in complete elimination of the risk of developing the disease or disorder.

An “increased” or “enhanced” amount (e.g., with respect to antitumor response, cancer cell metastasis) refers to an increase that is 1.1, 1.2, 1.3, 1.4, 1.5, 1.6, 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 2.1, 2.2, 2.3, 2.4, etc.) an amount or level described herein. It may also include an increase of at least 10%, at least 20%, at least 30%, at least 40%, at least 50%, at least 60%, at least 70%, at least 80%, at least 90%, at least 100%, at least 150%, at least 200%, at least 500%, or at least 1000% of an amount or level described herein.

A “decreased” or “reduced” or “lesser” amount (e.g., with respect to tumor size, cancer cell proliferation or growth) refers to a decrease that is about 1.1, 1.2, 1.3, 1.4, 1.5, 1.6 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 1.5, 1.6, 1.7, 1.8, etc.) an amount or level described herein. It may also include a decrease of at least 10%, at least 20%, at least 30%, at least 40%, at least 50%, at least 60%, at least 70%, at least 80%, or at least 90%, at least 100%, at least 150%, at least 200%, at least 500%, or at least 1000% of an amount or level described herein.

### A. Disorders

Methods and manufactured compositions disclosed herein find use in targeting a number of disorders, such as cellular proliferative disorders. A benefit of the approaches herein is that allogenic cells are used in combination with exogenous antibody administration to target specific proliferating cells targeted by the exogenous antibody. Unlike previous therapies, such as chemo or radiotherapy, using the approaches and pharmaceutical compositions herein, one is able to specifically target cells exhibiting detrimental proliferative activity, potentially without administering a systemic drug or toxin that impacts proliferating cells indiscriminately.

Examples of cellular proliferative and/or differentiative disorders include cancer, e.g., carcinoma, sarcoma, metastatic disorders or hematopoietic neoplastic disorders, e.g., leukemias. A metastatic tumor can arise from a multitude of primary tumor types, including but not limited to those of prostate, colon, lung, breast and liver origin.

As used herein, the terms “cancer”, “hyperproliferative” and “neoplastic” refer to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair.

The terms “cancer” or “neoplasms” include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the disease is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures.

The term “sarcoma” is art recognized and refers to malignant tumors of mesenchymal derivation.

Additional examples of proliferative disorders include hematopoietic neoplastic disorders. As used herein, the term “hematopoietic neoplastic disorders” includes diseases involving hyperplastic/neoplastic cells of hematopoietic origin, e.g., arising from myeloid, lymphoid or erythroid lineages, or precursor cells thereof. Preferably, the diseases arise from poorly differentiated acute leukemias, e.g., erythroblastic leukemia and acute megakaryoblastic leukemia. Additional exemplary myeloid disorders include, but are not limited to, acute promyeloid leukemia (APML), acute myelogenous leukemia (AML) and chronic myelogenous leukemia (CML) (reviewed in Vaickus, L. (1991) Crit Rev. in Oncol./Hemotol. 11:267-97); lymphoid malignancies include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease and Reed-Sternberg disease.

In some embodiments, the cancer is selected from the group consisting of: acute lymphoblastic leukemia (ALL), acute myeloid leukemia (AML), adrenocortical carcinoma, Kaposi sarcoma, AIDS-related lymphoma, primary CNS lymphoma, anal cancer, appendix cancer, astrocytoma, typical teratoid/rhabdoid tumor, basal cell carcinoma, bile duct cancer, bladder cancer, bone cancer, brain tumor, breast cancer, bronchial tumor, Burkitt lymphoma, carcinoid, cardiac tumors, medulloblastoma, germ cell tumor, primary CNS lymphoma, cervical cancer, cholangiocarcinoma, chordoma, chronic lymphocytic leukemia (CLL), chronic myelogenous leukemia (CML), chronic myeloproliferative neoplasms, colorectal cancer, craniopharyngioma, cutaneous T-cell lymphoma, ductal carcinoma in situ, embryonal tumors, endometrial cancer, ependymoma, esophageal cancer, esthesioneuroblastoma, Ewing sarcoma, extracranial germ cell tumor, extragonadal germ cell tumor, eye cancer (e.g., intraocular melanoma or retinoblastoma), fallopian tube cancer, fibrous histiocytoma of bone, osteosarcoma, gallbladder cancer, gastric cancer, gastrointestinal carcinoid tumor, gastrointestinal stromal tumors (GIST), germ cell tumors, gestational trophoblastic disease, hairy cell leukemia, head and neck cancer, heart tumor, hepatocellular cancer, histiocytosis, Hodgkin lymphomas, hypopharyngeal cancer, intraocular melanoma, islet cell tumors, pancreatic neuroendocrine tumors, kidney (renal cell) carcinoma, Langerhans cell histiocytosis, laryngeal cancer, leukemia, lip and oral cavity cancer, liver cancer, lung cancer (e.g., non-small cell lung cancer, small cell lung cancer, pleuropulmonary blastoma, and tracheobronchial tumor), lymphoma, male breast cancer, malignant fibrous histiocytoma of bone, melanoma, Merkel cell carcinoma, mesothelioma, metastatic cancer, metastatic squamous neck cancer, midline tract carcinoma, mouth cancer, multiple endocrine neoplasia syndromes, multiple myeloma/plasma cell neoplasms, mycosis fungoides, myelodysplastic syndromes, myelodysplastic/myeloproliferative neoplasms, myeloproliferative neoplasms, nasal cavity and paranasal sinus cancer, nasopharyngeal cancer, neuroblastoma, non-Hodgkin lymphoma, oral cancer, lip and oral cavity cancer, oropharyngeal cancer, osteosarcoma, malignant fibrous histiocytoma, ovarian cancer, pancreatic cancer, pancreatic neuroendocrine tumors, papillomatosis, paraganglioma, paranasal sinus and nasal cavity cancer, parathyroid cancer, penile cancer, pharyngeal cancer, pheochromocytomas, pituitary tumor, plasma cell neoplasm, multiple myeloma, pleuropulmonary blastoma, pregnancy and breast cancer, primary central nervous system lymphoma, primary peritoneal cancer, prostate cancer, rectal cancer, recurrent cancer, renal cell cancer, retinoblastoma, rhabdomyosarcoma, salivary gland cancer, sarcoma (e.g., childhood rhabdomyosarcoma, childhood vascular tumors. Ewing sarcoma. Kaposi sarcoma, osteosarcoma, soft tissue sarcoma, uterine sarcoma), Sezary syndrome, skin cancer, small intestine cancer, soft tissue sarcoma, squamous cell carcinoma, squamous neck cancer, stomach cancer, T-cell lymphomas, testicular cancer, throat cancer, nasopharyngeal cancer, oropharyngeal cancer, hypopharyngeal cancer, thryomoma and thymic carcinomas, thyroid cancer, tracheobronchial tumors, transitional cell cancer of the renal pelvis and ureter, urethral cancer, uterine cancer, uterine sarcoma, vaginal cancer, vascular tumors, vulvar cancer, and Wilms tumor.

In some embodiments, the cancer is a solid tumor.

In some embodiments, the cancer is metastatic.

In some embodiments, the cancer is a CD30+ cancer.

In some embodiments, the CD30+ cancer is a lymphoma. In some embodiments, the lymphoma is selected from the group consisting of classic Hodgkin lymphoma (CHL), anaplastic large cell lymphoma (ALCL), grey zone lymphoma (GZL), Epstein-Barr virus-positive diffuse large B-cell lymphoma (EBV+DLBCL), and combinations thereof.

In some embodiments, the cancer is selected from the group consisting of Hodgkin lymphoma, non-Hodgkin lymphoma, B-cell non-Hodgkin lymphoma, peripheral T-cell lymphoma, peripheral T-cell lymphoma not otherwise specified, cutaneous T cell lymphoma, anaplastic large-cell lymphoma, CD30⁺ B-cell lymphoma, multiple myeloma, my cosis fungoides, and leukemia. In some embodiments, the patient has relapsed disease. In some embodiments, the patient is refractory to previous therapeutic interventions.

In some embodiments, the disorder is relapsed or refractory classical Hodgkin lymphoma (cHL).

In some embodiments, the disorder is relapsed or refractory peripheral T-cell lymphoma (PTCL). In some embodiments, the PTCL is a PTCL subtype selected from the group consisting of Peripheral T-cell lymphoma not otherwise specified, angioimmunoblastic T-cell lymphoma, anaplastic large-cell lymphoma anaplastic lymphoma kinase (ALK)-positive, and anaplastic large-cell lymphoma ALK-negative.

### B. Patients

Suitable patients for the compositions and methods herein include those who are suffering from, who have been diagnosed with, or who are suspected of having a cellular proliferative and/or differentiative disorder, e.g., a cancer. Patients subjected to technology of the disclosure herein generally respond better to the methods and compositions herein, in part because the pharmaceutical compositions are allogeneic and target cells identified by the antibodies, rather than targeting proliferating cells generally. As a result, there is less off-target impact and the patients are more likely to complete treatment regimens without substantial detrimental off-target effects.

In some embodiments, the methods of treatment provided herein may be used to treat a subject (e.g., human, monkey, dog, cat, mouse) who has been diagnosed with or is suspected of having a cellular proliferative and/or differentiative disorder, e.g., a cancer. In some embodiments, the subject is a mammal. In some embodiments, the subject is a human.

As used herein, a subject refers to a mammal, including, for example, a human.

In some embodiments, the mammal is selected from the group consisting of an armadillo, an ass, a bat, a bear, a beaver, a cat, a chimpanzee, a cow, a coyote, a deer, a dog, a dolphin, an elephant, a fox, a panda, a gibbon, a giraffe, a goat, a gopher, a hedgehog, a hippopotamus, a horse, a humpback whale, a jaguar, a kangaroo, a koala, a leopard, a lion, a llama, a lynx, a mole, a monkey, a mouse, a narwhal, an orangutan, an orca, an otter, an ox, a pig, a polar bear, a porcupine, a puma, a rabbit, a raccoon, a rat, a rhinoceros, a sheep, a squirrel, a tiger, a walrus, a weasel, a wolf, a zebra, a goat, a horse, and combinations thereof.

In some embodiments, the mammal is a human.

The subject, e.g., the human subject, can be a child, e.g., from or from about 0 to or to about 14 years in age. The subject can be a youth, e.g., from or from about 15 to or to about 24 years in age. The subject can be an adult, e.g., from or from about 25 to or to about 64 years in age. The subject can be a senior, e.g. 65+ years in age.

In some embodiments, the subject may be a human who exhibits one or more symptoms associated with a cellular proliferative and/or differentiative disorder, e.g., a cancer, e.g., a tumor. Any of the methods of treatment provided herein may be used to treat cancer at various stages. By way of example, the cancer stage includes but is not limited to early, advanced, locally advanced, remission, refractory, reoccurred after remission and progressive. In some embodiments, the subject is at an early stage of a cancer. In other embodiments, the subject is at an advanced stage of cancer. In various embodiments, the subject has a stage I, stage II, stage III or stage IV cancer. The methods of treatment described herein can promote reduction or retraction of a tumor, decrease or inhibit tumor growth or cancer cell proliferation, and/or induce, increase or promote tumor cell killing. In some embodiments, the subject is in cancer remission. The methods of treatment described herein can prevent or delay metastasis or recurrence of cancer.

In some embodiments, the subject is at risk, or genetically or otherwise predisposed (e.g., risk factor), to developing a cellular proliferative and/or differentiative disorder, e.g., a cancer, that has or has not been diagnosed.

As used herein, an “at risk” individual is an individual who is at risk of developing a condition to be treated, e.g., a cellular proliferative and/or differentiative disorder, e.g., a cancer. Generally, an “at risk” subject may or may not have detectable disease, and may or may not have displayed detectable disease prior to the treatment methods described herein. “At risk” denotes that an individual has one or more so-called risk factors, which are measurable parameters that correlate with development of a disease or condition and are known in the art. For example, an at risk subject may have one or more risk factors, which are measurable parameters that correlate with development of cancer. A subject having one or more of these risk factors has a higher probability of developing cancer than an individual without these risk factor(s). In general, risk factors may include, for example, age, sex, race, diet, history of previous disease, presence of precursor disease, genetic (e.g., hereditary) considerations, and environmental exposure. In some embodiments, the subjects at risk for cancer include, for example, those having relatives who have experienced the disease, and those whose risk is determined by analysis of genetic or biochemical markers.

In addition, the subject may be undergoing one or more standard therapies, such as chemotherapy, radiotherapy, immunotherapy, surgery, or combination thereof. Accordingly, one or more kinase inhibitors may be administered before, during, or after administration of chemotherapy, radiotherapy, immunotherapy, surgery or combination thereof.

In certain embodiments, the subject may be a human who is (i) substantially refractory to at least one chemotherapy treatment, or (ii) is in relapse after treatment with chemotherapy, or both (i) and (ii). In some of embodiments, the subject is refractory to at least two, at least three, or at least four chemotherapy treatments (including standard or experimental chemotherapies).

In some embodiments, the subject has relapsed after treatment with or is refractory to an anti-CD30 antibody. In some embodiments, the anti-CD30 antibody is brentuximab bedotin.

In some embodiments, the subject has experienced disease progression after treatment with autologous stem cell transplant or chimeric antigen receptor T-cell therapy (CAR-T)

In some embodiments, the patient is diagnosed with or has been diagnosed with CD30+ cancer.

In some embodiments, the patient is diagnosed with or has been diagnosed with a CD30+ cancer by immunohistochemical staining of a biopsy or surgical sample of the cancer. In some embodiments, the patient is diagnosed with or has been diagnosed with a CD30+ cancer by chromogenic in situ hybridization. In some embodiments, the patient is diagnosed with or has been diagnosed with a CD30+ cancer by fluorescent in situ hybridization of a biopsy or surgical sample of the cancer. In some embodiments, the patient is diagnosed with or has been diagnosed with a CD30+ cancer by genetic analysis.

In some embodiments, the patient is refractory to or has a recurrence after treatment with a CD30 inhibitor.

In some embodiments, the patient is refractory to or has a recurrence after treatment with a chemotherapy drug.

In some embodiments, the chemotherapy drug is selected from the group consisting of cisplatin, docetaxel, carboplatin, gemcitabine, cisplatin, pemetrexed, or combinations thereof.

In some embodiments, the patient is refractory to or has a recurrence after treatment with a tyrosine kinase inhibitor. In some embodiments, the tyrosine kinase inhibitor is selected from the group consisting of gefitinib, erlontinib, afatinib, osimertinib, and combinations thereof.

In some embodiments, the patient has relapsed or refractory classical Hodgkin lymphoma and has received at least two lines of therapy including one prior line of combination chemotherapy. In some embodiments, the prior therapy comprises brentuximab vedotin and a check point inhibitor.

In some embodiments, the patient has relapsed or refractory peripheral T-cell lymphoma and has received at least one prior line of combination therapy. In some embodiments, the prior therapy comprises brentuximab vedotin. In some embodiments, the patient has relapsed or refractory peripheral T-cell lymphoma and is or has been intolerant to brentuximab vedotin.

### C. Lymphodepletion

In some embodiments, the patient is lymphodepleted before treatment.

Illustrative lymphodepleting chemotherapy regimens, along with correlative beneficial biomarkers, are described in WO 2016/191756 and WO 2019/079564, hereby incorporated by reference in their entirety. In certain embodiments, the lymphodepleting chemotherapy regimen comprises administering to the patient doses of cyclophosphamide (between 200 mg/m²/day and 2000 mg/m²/day) and doses of fludarabine (between 20 mg/m²/day and 900 mg/m²/day).

In some embodiments, lymphodepletion comprises administration of or of about 250 to about 500 mg/m² of cyclophosphamide, e.g., from or from about 250 to or to about 500, 250, 400, 500, about 250, about 400, or about 500 mg/m² of cyclophosphamide.

In some embodiments, lymphodepletion comprises administration of or of about 20 mg/m²/day to or to about 40 mg/m²/day fludarabine, e.g., 30 or about 30 mg/m²/day.

In some embodiments, lymphodepletion comprises administration of both cyclophosmamide and fludarabine.

In some embodiments, the patient is lymphodepleted by intravenous administration of cyclophosphamide (250 mg/m²/day) and fludarabine (30 mg/m²/day).

In some embodiments, the patient is lymphodepleted by intravenous administration of cyclophosphamide (500 mg/m²/day) and fludarabine (30 mg/m²/day).

In some embodiments, the lymphodepletion occurs no more than 5 days prior to the first dose of NK cells. In some embodiments, the lymphodepletion occurs no more than 7 days prior to the first dose of NK cells.

In some embodiments, lymphodepletion occurs daily for 3 consecutive days, starting 5 days before the first dose of NK cells (i.e., from Day-5 through Day-3).

In some embodiments, the lymphodepletion occurs on day-5, day-4 and day-3.

### D. Administration

### 1. NK Cells

In some embodiments, the NK cells are administered to the patient as part of a pharmaceutical composition, e.g., a pharmaceutical composition described herein. Cells are administered after thawing, in some cases without any further manipulation in cases where their cryoprotectant is compatible for immediate administration. For a given individual, a treatment regimen often comprises administration over time of multiple aliquots or doses of NK cells drawn from a common batch or donor.

In some embodiments, treatment comprises administration of doses of NK cells (e.g., as described herein), drawn from a common batch, master cell bank, or donor. In some embodiments, treatment comprises administration of doses of NK cells (e.g., as described herein), drawn from different batches, master cell banks, or donors. For example, a patient initially dosed with NK cells produced from a first donor can be dosed with NK cells produced from a second donor if the patient develops immunogenicity against the NK cells produced from the first donor.

The NK cells can be administered by any suitable means, for example, by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration. In some embodiments, a given dose is administered by a single bolus administration of the cells. In some embodiments, a given dose is administered by multiple bolus administrations of the cells, for example, over a period of no more than 3 days, or by continuous infusion administration of the cells. In some embodiments, administration of the cell dose or any additional therapies, e.g., the multispecific engager therapy, the lymphodepleting therapy, intervention therapy and/or combination therapy, is carried out via outpatient delivery.

In the context of adoptive cell therapy, administration of a given “dose” can encompass administration of the given amount or number of cells as a single composition and/or single uninterrupted administration, e.g., as a single injection or continuous infusion. A given “dose” can also encompass administration of the given amount or number of cells as a split dose or as a plurality of compositions, provided in multiple individual compositions or infusions, over a specified period of time, such as over no more than 3 days. Thus, in some contexts, the dose is a single or continuous administration of the specified number of cells, given or initiated at a single point in time. In some contexts, however, the dose is administered in multiple injections or infusions over a period of no more than three days, such as once a day for three days or for two days or by multiple infusions over a single day period.

In some embodiments, the NK cells are administered to the patient at a range of at or about 1 million to at or about 100 billion cells. In some embodiments, the NK cells, e.g., the NK cells described herein, are administered at or at about 5×10⁶ to or to about 1×10⁹ NK cells per dose. In some embodiments, the NK cells are administered at or at about 5×10⁶, at or at about 1×10⁷, at or at about 3×10⁷, at or at about 1×10⁸, at or at about 3×10⁸, or at or at about 1×10⁹ cells per dose. In some embodiments, the NK cells are administered at or about 1 million to at or about 20 billion cells (e.g., at or about 5 million cells, at or about 25 million cells, at or about 50 million cells, at or about 75 million cells, at or about 100 million cells, at or about 200 million cells, at or about 300 million cells, at or about 400 million cells, at or about 500 million cells, at or about 1 billion cells, at or about 2 billion cells, at or about 3 billion cells, at or about 4 billion cells, at or about 5 billion cells, at or about 6 billion cells, at or about 7 billion cells, at or about 8 billion cells, at or about 9 billion cells, at or about 10 billion cells, or a range defined by any two of the foregoing values), at or about 10 million to at or about 20 billion cells (e.g., at or about 25 million cells, at or about 50 million cells, at or about 75 million cells, at or about 100 million cells, at or about 200 million cells, at or about 300 million cells, at or about 400 million cells, at or about 500 million cells, at or about 1 billion cells, at or about 2 billion cells, at or about 3 billion cells, at or about 4 billion cells, at or about 5 billion cells, at or about 6 billion cells, at or about 7 billion cells, at or about 8 billion cells, at or about 9 billion cells, at or about 10 billion cells, at or about 20 billion cells, or a range defined by any two of the foregoing values), and in some cases at or about 100 million cells to at or about 50 billion cells (e.g., at or about 150 million cells, at or about 200 million cells, at or about 300 million cells, at or about 400 million cells, at or about 500 million cells, at or about 1 billion cells, at or about 2 billion cells, at or about 3 billion cells, at or about 4 billion cells, at or about 5 billion cells, at or about 6 billion cells, at or about 7 billion cells, at or about 8 billion cells, at or about 9 billion cells, at or about 10 billion cells, at or about 20 billion cells, at or about 30 billion cells, at or about 40 billion cells), or any value in between any of these ranges, per dose.

Thus, in some embodiments, the NK cells are administered in a dose comprising at or at about 1×10⁶, 2×10⁶, 3×10⁶, 4×10⁶, 5×10⁶, 6×10⁶, 7×10⁶, 8×10⁶, or 9×10⁶ cells per dose, at or at about 1×10⁷, 2×10⁷, 3×10⁷, 4×10⁷, 5×10⁷, 6×10⁷, 7×10⁷, 8×10⁷, or 9×10⁷ cells per dose, at or at about 1×10⁸, 2×10⁸, 3×10⁸, 4×10⁸, 5×10⁸, 6×10⁸, 7×10⁸, 8×10⁸, or 9×10⁸ cells per dose, at or at about 1×10⁹, 2×10⁹, 3×10⁹, 4×10⁹, 5×10⁹, 6×10⁹, 7×10⁹, 8×10⁹, or 9×10⁹ cells per dose, at or at about 1×10¹⁰ or 2×10¹⁰ cells per dose.

Thus, in some embodiments, the NK cells are administered in a dose comprising at least or at least about 1×10⁶, 2×10⁶, 3×10⁶, 4×10⁶, 5×10⁶, 6×10⁶, 7×10⁶, 8×10⁶, or 9×10⁶ cells per dose, at least or at least about 1×10⁷, 2×10⁷, 3×10⁷, 4×10⁷, 5×10⁷, 6×10⁷, 7×10⁷, 8×10⁷, or 9×10⁷ cells per dose, at least or at least about 1×10⁸, 2×10⁸, 3×10⁸, 4×10⁸, 5×10⁸, 6×10⁸, 7×10⁸, 8×10⁸, or 9×10⁸ cells per dose, at least or at least about 1×10⁹, 2×10⁹, 3×10⁹, 4×10⁹, 5×10⁹, 6×10⁹, 7×10⁹, 8×10⁹, or 9×10⁹ cells per dose, or at least or at least about 1×10¹⁰, or 2×10¹⁰ cells per dose.

In some embodiments, the dose of cells is a flat dose of cells or fixed dose of cells such that the dose of cells is not tied to or based on the body surface area or weight of a patient.

In some embodiments, the dose of cells is administered based on the weight of the patient. For example, the dose can be determined per kilogram of body weight of the patient. In some embodiments, the dose of cells comprises between at or about 1×10⁵ of the cells/kg and at or about 1×10⁸ of the cells/kg, such as between at or about 1.5×10⁵ of the cells/kg and at or about 1.5×10⁷ of the cells/kg, or between at or about 4×10⁵ of the cells/kg and at or about 4×10⁶ of the cells/kg.

Thus, in some embodiments, the NK cells are administered in a dose comprising at or at about 1×10⁵, 1.5×10⁵, 2×10⁵, 2.5×10⁵, 3×10⁵, 3.5×10⁵, 4×10⁵, 4.5×10⁵, 5×10⁵, 5.5×10⁵, 6×10⁵, 6.5×10⁵, 7×10⁵, 7.5×10⁵, 8×10⁵, 8.5×10⁵, 9×10⁵, or 9.5×10⁵ cell/kg, 1×10⁶, 1.5×10⁶, 2×10⁶, 2.5×10⁶, 3×10⁶, 3.5×10⁶, 4×10⁶, 4.5×10⁶, 5×10⁶, 5.5×10⁶, 6×10⁶, 6.5×10⁶, 7×10⁶, 7.5×10⁶, 8×10⁶, 8.5×10⁶, 9×10⁶, or 9.5×10⁶ cells/kg, at or at about 1×10⁷, 1.5×10⁷, 2×10⁷, 2.5×10⁷, 3×10⁷, 3.5×10⁷, 4×10⁷, 4.5×10⁷, 5×10⁷, 5.5×10⁷, 6×10⁷, 6.5×10⁷, 7×10⁷, 7.5×10⁷, 8×10⁷, 8.5×10⁷, 9×10⁷ cells/kg, or 9.5×10⁷, or at or at about 1×10⁸, 1.5×10⁸, or 2×10⁸ cells/kg.

In some embodiments, the NK cells are administered in a dose comprising at least or at least about 1×10⁵, 1.5×10⁵, 2×10⁵, 2.5×10⁵, 3×10⁵, 3.5×10⁵, 4×10⁵, 4.5×10⁵, 5×10⁵, 5.5×10⁵, 6×10⁵, 6.5×10⁵, 7×10⁵, 7.5×10⁵, 8×10⁵, 8.5×10⁵, 9×10⁵, or 9.5×10⁵ cell/kg, at least or at least about 1×10⁶, 1.5×10⁶, 2×10⁶, 2.5×10⁶, 3×10⁶, 3.5×10⁶, 4×10⁶, 4.5×10⁶, 5×10⁶, 5.5×10⁶, 6×10⁶, 6.5×10⁶, 7×10⁶, 7.5×10⁶, 8×10⁶, 8.5×10⁶, 9×10⁶, or 9.5×10⁶ cells/kg, at least or at least about 1×10⁷, 1.5×10⁷, 2×10⁷, 2.5×10⁷, 3×10⁷, 3.5×10⁷, 4×10⁷, 4.5×10⁷, 5×10⁷, 5.5×10⁷, 6×10⁷, 6.5×10⁷, 7×10⁷, 7.5×10⁷, 8×10⁷, 8.5×10⁷, 9×10⁷ cells/kg, or 9.5×10⁷, or at least or at least about 1×10⁸ or 1.5×10⁸ cells/kg.

In some embodiments, the dose of cells, e.g., NK cells, is administered to the subject as a single dose or is administered only one time within a period of two weeks, one month, three months, six months, 1 year or more.

The ability to offer repeat dosing may allow patients to experience or maintain a deeper or prolonged response from the therapy. Thus, in some embodiments, the patient receives multiple doses, e.g., two or more doses or at least one subsequent dose, of the NK cells. In some embodiments, two, three, four, five, six, seven, eight, nine, or ten doses are administered to a subject. In some embodiments, the at least one subsequent dose comprises a second dose. In some embodiments, the at least one subsequent dose comprises a second dose and a third dose. In some embodiments, the at least one subsequent dose comprises a second, third, and fourth dose. In some embodiments, the at least one subsequent dose comprises a second, third, fourth, and fifth dose. In some embodiments, the at least one subsequent dose comprises a second, third, fourth, fifth, and sixth dose. In some embodiments, the at least one subsequent dose comprises a second, third, fourth, fifth, sixth, and seventh dose. In some embodiments, the at least one subsequent dose comprises a second, third, fourth, fifth, sixth, seventh, and eighth dose. In some embodiments, a patient can receive response-based dosing, during which the patient continues to receive doses of NK cell therapy for as long as the patient derives a benefit. The number of doses and the number of cells administered in each dose can also be tailored to the individual patient. In some embodiments, the number of cells administered to the subject in the additional or subsequent dose or doses are the same as or similar to the first dose. Thus, the NK cell therapies described herein can be tailored to each patient based on that patient's own response. In some cases, the therapy can be terminated if the patient no longer derives a benefit from the NK cell therapy. In some cases, the therapy can also be reinitiated if the patient relapses.

In some embodiments involving multiple doses or repeat dosing, the NK cells are administered weekly, bi-weekly, once every three weeks, once every four weeks, once every five weeks, once every six weeks, once every seven weeks, once every eight weeks, once every nine weeks, once every ten weeks, once every 11 weeks, once every 12 weeks, once every 13 weeks, once every 14 weeks, once every 15 weeks, or once every 16 weeks. For example, a patient can receive a first dose, a second dose, and a third dose, wherein each dose is separated by or by one week. In some embodiments, the NK cells are administered monthly. In some embodiments, the NK cells are administered every other month or once every three months. In some embodiments, the NK cells are administered for or for about three weeks. In some embodiments, the NK cells are administered for or for about four weeks. In some embodiments, the NK cells are administered for or for about 8 weeks.

In some embodiments, the dosing schedule can vary over the course of the therapy. Thus, for example, the patient can receive a first series (or cycle) of doses every 1, 2, 3, 4, 5, 6, 7, 8, 9, 10, 11, 12, 13, 14, 15, or 16 weeks and a second series of doses every 1, 2, 3, 4, 5, 6, 7, 8, 9, 10, 11, 12, 13, 14, 15, or 16 weeks, wherein the time between the first series of doses and the second series of doses is different. In some embodiments, the patient receives 1, 2, 3, 4, 5, 6, 7, 8, 9, or 10 doses in the first series of doses and 1, 2, 3, 4, 5, 6, 7, 8, 9, or 10 doses in the second series of doses, wherein the number of doses in the first series and the second series of doses can be the same or different. Thus, for example, a patient may receive four doses administered every other week in the first series of doses and four doses administered every 12 weeks in the second series of doses.

In some embodiments, the NK cells are administered between one and four times over the course of nine months.

In some embodiments, the NK cells are cryopreserved in an infusion-ready media, e.g., a cryopreservation composition suitable for intravenous administration, e.g., as described herein.

In some embodiments, the NK cells are cryopreserved in vials containing from or from about 1×10⁷ to or to about 1×10⁹ cells per vial. In some embodiments, the NK cells are cryopreserved in vials containing a single dose.

In some embodiments, the cells are thawed, e.g., in a 37° C. water bath, prior to administration.

In some embodiments, the thawed vial(s) of NK cells are aseptically transferred to a single administration vessel, e.g., administration bag using, e.g., a vial adapter and a sterile syringe. The NK cells can be administered to the patient from the vessel through a Y-type blood/solution set filter as an IV infusion, by gravity.

In some embodiments, the NK cells are administered as soon as practical, preferably less than 90 minutes, e.g., less than 80, 70, 60, 50, 40, 30, 20, or 10 minutes after thawing. In some embodiments, the NK cells are administered within 30 minutes of thawing.

In some embodiments, the pharmaceutical composition is administered intravenously via syringe.

In some embodiments, 1 mL, 4 mL, or 10 mL of drug product is administered to the patient intravenously via syringe.

In some embodiments, the patient is administered acetaminophen prior to being administered the NK cell infusion. In some embodiments, the patient is administered 100, 200, 250, 300, 400, 500, 600, 700, 750, 800, 900, 1000, 1100, 1200, 1250, 1300, 1400, 1500, 1600, 1700, 1750, 1800, 1900, or 2000 mg of acetaminophen. In some embodiments, the acetaminophen is administered to the patient immediately prior to the NK cells. In some embodiments, the acetaminophen is administered 5, 10, 15, 20, 25, 30, 35, 40, 45, 50, 55, 60, 70, 80, 90, 100, 110, 120, 150, or 180 minutes before the NK cells. In some embodiments, the acetaminophen is administered orally.

In some embodiments, the patient is administered diphenhydramine prior to being administered the NK cell infusion. In some embodiments, the patient is administered 5, 10, 12.5, 15, 17.5, 20, 25, 30, 40, 50, 60, 70, 75, 80, 90, or 100 mg of diphenhydramine. In some embodiments, the diphenhydramine is administered to the patient immediately prior to the NK cells. In some embodiments, the diphenhydramine is administered 5, 10, 15, 20, 25, 30, 35, 40, 45, 50, 55, 60, 70, 80, 90, 100, 110, 120, 150, or 180 minutes before the NK cells. In some embodiments, the acetaminophen is administered orally.

In some embodiments, the patient is monitored for a period of time before and after administration of NK cells. For example, the patient's vital signs can be monitored. These can include temperature, respiratory rate, heart rate, blood pressure, and oxygen saturation (SaO₂) by pulse oximetry. In some cases, at least one vital sign is measured beginning 5, 10, 15 20, 25, or 30 minutes prior to NK cell administration. After NK cell administration, at least one vital sign can be monitored for 1, 2, 3, 4, or 5 hours continuously or at regular or irregular intervals, including approximately every 5, 10, 15, 20, 25, or 30 minutes. In some cases, vital signs can be monitored after NK cell administration until the patient is stable.

### 2. Multispecific Engagers

In some embodiments, the NK cell(s) described herein, e.g., the pharmaceutical compositions comprising NK cell(s) described herein, are administered to the patient in combination with a multispecific engager, e.g., a multispecific engager described herein, e.g., an CD30-targeting multispecific engager. In some embodiments, a multispecific engager is administered together with the NK cells as part of a pharmaceutical composition. In some embodiments, a multispecific engager is administered separately from the NK cells, e.g., as part of a separate pharmaceutical composition.

Multispecific engagers can be administered prior to, subsequent to, or simultaneously with administration of the NK cells.

In some embodiments, the multispecific engager is administered before the NK cells. In some embodiments, the multispecific engager is administered after the NK cells.

In some embodiments, the NK cells are administered the day after the multispecific engager is administered.

In some embodiments, the NK cells are administered at each administration, while the multispecific engager is administered at a subset of the administrations. For example, in some embodiments, the NK cells are administered once a week and the multispecific engager is administered once a month.

In some embodiments, a dose of multispecific engager is given prior to the first dose of cells. In some embodiments, a debulking dose of the multispecific engager is given prior to the first dose of cells.

In some embodiments, the multispecific engager is administered to the patient at or at about 0.01 to 10 mg/kg, e.g., at or at about 0.1 to 9, 0.01 to 8, 0.01 to 7, 0.01 to 6, 0.01 to 5, 0.01 to 4, 0.01 to 3, 0.01 to 2, 0.01 to 1.5, 0.01 to 1, 0.01 to 0.5, 0.01 to 0.15, 0.01 to 0.04, 0.04 to 10, 0.04 to 9, 0.04 to 8, 0.04 to 7, 0.04 to 6, 0.04 to 5, 0.04 to 4, 0.04 to 3, 0.04 to 2, 0.04 to 1.5, 0.04 to 1, 0.04 to 0.5, 0.04 to 0.15, 0.15 to 10, 0.15 to 9, 0.15 to 8, 0.15 to 7, 0.15 to 6, 0.15 to 5, 0.15 to 4, 0.15 to 3, 0.15 to 2, 0.15 to 1.5, 0.15 to 1, 0.15 to 0.5, 0.5 to 10, 0.5 to 9, 0.5 to 8, 0.5 to 7, 0.5 to 6, 0.5 to 5, 0.5 to 4, 0.5 to 3, 0.5 to 2, 0.5 to 1.5, 0.5 to 1, 1 to 10, 1 to 9, 1 to 8, 1 to 7, 1 to 6, 1 to 5, 1 to 4, 1 to 3, 1 to 2, 1 to 1.5, 1.5 to 10, 1.5 to 9, 1.5 to 8, 1.5 to 7, 1.5 to 6 1.5 to 5, 1.5 to 4, 1.5 to 3, 1.5 to 2, 2 to 10, 2 to 9, 2 to 8, 2 to 7, 2 to 6, 2 to 5, 2 to 4, 2 to 3, 3 to 10, 3 to 9, 3 to 8, 3 to 7, 3 to 6, 3 to 5, 3 to 4, 4 to 10, 4 to 9, 4 to 8, 4 to 7, 4 to 6, 4 to 5, 5 to 10, 5 to 9, 5 to 8, 5 to 7, 5 to 6, 6 to 10, 6 to 9, 6 to 8, 6 to 7, 7 to 10, 7 to 9, 7 to 8, 8 to 10, 8 to 9, or 9 to 10 mg/kg.

In some embodiments, the multispecific engager is administered to the patient at or at about 0.01 mg/kg, 0.04 mg/kg, 0.015 mg/kg, 0.5 mg/kg, 1.0 mg/kg, 1.5 mg/kg, 3.0 mg/kg, 4.5 mg/kg, or 7.0 mg/kg.

In some embodiments, a dose of the multispecific engager comprises from or from about 100 to or to about 300 mg, e.g., from or from about 100 to 275, 100 to 250, 100 to 225, 100 to 200, 100 to 175, 100 to 150, 100 to 125, 125 to 300, 125 to 275, 125 to 250, 125 to 225, 125 to 200, 125 to 175, 125 to 150, 150 to 300, 150 to 275, 150 to 250, 150 to 225, 150 to 200, 150 to 175, 175 to 300, 175 to 275, 175 to 250, 175 to 225, 175 to 200, 200 to 300, 200 to 275, 200 to 250, 200 to 225, 225 to 300, 225 to 275, 225 to 250, 250 to 275, or 275 to 300 mg.

The multispecifc engager can be administered by any suitable means, for example, by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, the multispecific engager is administered by parenteral, intrapulmonary, and intranasal, and, if desired for local treatment, intralesional administration. Parenteral infusions include intramuscular, intravenous, intraarterial, intraperitoneal, or subcutaneous administration. In some embodiments, a given dose is administered by a single bolus administration of the multispecific engager. In some embodiments, a given dose is administered by multiple bolus administrations of the multispecific engager, for example, over a period of no more than 3 days, or by continuous infusion administration of the cells.

In some embodiments, the multispecific engager is administered prior to administration of the NK cells. In some embodiments, administration of the multispecific engager is completed at least 15, 30, 45, 60, 90, or 120 minutes prior to initiation of the administration of the NK cells. In some embodiments, the NK cells are administered prior to administration of the multispecific engager. In some embodiments, administration of the NK cells is completed at least 15, 30, 45, 60, 90, or 120 minutes prior to initiation of the administration of the multispecific engager. In some embodiments, the multispecific engager is administered at a rate such that the dose is administered over 1, 2, 3, 4, 5, 6, 7, or 8 hours. In some embodiments, the multispecific engager is administered at a rate such that the dose is administered over 4 hours.

In some embodiments, the multispecific engager is administered weekly for six weeks and the NK cells are administered weekly for four weeks. In some embodiments, the multispecific engager will be administered at a fixed dose of 200 mg IV QW on day 1, 8, 15, 22, 29, and 36 of each cycle over 4 hours. In some embodiments, the NK cells will be administered at a dose of one billion or four billion cells on day 1, 8, and 15 of each cycle. In some embodiments, the patient will be administered one, two, three, four, or five cycles of therapy. In some embodiments, there will be a one, two, three, four, or five week break between cycles of therapy.

### 3. Cytokines

In some embodiments, a cytokine is administered to the patient.

In some embodiments, the cytokine is administered together with the NK cells as part of a pharmaceutical composition. In some embodiments, the cytokine is administered separately from the NK cells, e.g., as part of a separate pharmaceutical composition.

In some embodiments, the cytokine is IL-2.

In some embodiments, the IL-2 is administered subcutaneously.

In some embodiments, the IL-2 is administered from between 1 to 4 or about 1 to about 4 hours following the conclusion of NK cell administration. In some embodiments, the IL-2 is administered at least 1 hour following the conclusion of NK cell administration. In some embodiments, the IL-2 is administered no more than 4 hours following the conclusion of NK cell administration. In some embodiments, the IL-2 is administered at least 1 hour after and no more than 4 hours following the conclusion of NK cell administration.

In some embodiments, the IL-2 is administered at up to 10 million IU/M², e.g., up to 1 million, 2 million, 3 million, 4 million, 5 million, 6 million, 7 million, 8 million, 9 million, or 10 million IU/m².

In some embodiments, the IL-2 is administered at or at about 1 million, at or at about 2 million, at or at about 3 million, at or at about 4 million, at or at about 5 million, at or at about 6 million, at or at about 7 million, at or at about 8 million, at or at about 9 million, at or at about 10 million IU/M²

In some embodiments, the IL-2 is administered at or at about 1×10⁶ IU/M². In some embodiments, the IL-2 is administered at or at about 2×10⁶ IU/M².

In some embodiments, less than 1×10⁶ IU/M² IL-2 is administered to the patient.

In some embodiments, a flat dose of IL-2 is administered to the patient. In some embodiments, a flat dose of 6 million IU or about 6 million IU is administered to the patient.

In some embodiments, IL-2 is not administered to the patient.

### 4. Pre-Treatments

In some embodiments, the patient is pre-treated with medication. In some embodiments, the medication is selected from the group consisting of an H1 antagonist, an H2 antagonist, acetaminophen, a prophylactic antiemetic, and combinations thereof. In some embodiments, the H1 antagonist is diphenhydramine. In some embodiments, the H2 antagonist is famotidine.

### E. Treatment Cycles and Regimens

In some embodiments, the NK cells and multispecific engagers described herein are administered to the patient as part of a treatment cycle that spans multiple days. In some embodiments, the NK cells and multispecific engagers are administered to the patient as part of a treatment regimen that comprises one or more treatment cycles.

In some embodiments, the treatment regimen continues until the patient's disorder (e.g., CD30⁺ cancer) progresses, or until the doses are discontinued due to the patient's intolerance of the NK cell, the multispecific engager, or both, or until the patient experiences toxicity the NK cells, the multispecific engager, or both

In some embodiments, the treatment regimen comprises a treatment break (e.g., a period without administration of the NK cells or multispecific engagers) between treatment cycles. In some embodiments, the treatment break is from 1 to 8 weeks, e.g., from 1 to 8, 1 to 7, 1 to 6, 1 to 5, 1 to 4, 1 to 3, 1 to 2, 2 to 8, 2 to 7, 2 to 6, 2 to 5, 2 to 4, 2 to 3, 3 to 8, 3 to 7, 3 to 6, 3 to 5, 3 to 4, 4 to 8, 4 to 7, 4 to 6, 4 to 5, 5 to 8, 5 to 7, 5 to 6, 6 to 8, 6 to 7, or 7 to 8 weeks. In some embodiments, the treatment break is at least 1, 2, 3, 4, 5, 6, 7, or 8 weeks. In some embodiments, the treatment break is or is about 1, 2, 3, 4, 5, 6, 7, or 8 weeks.

In some embodiments, the treatment cycle is from 2 to 60 days, e.g., from 2 to 50, 2 to 40, 2 to 30, 2 to 20, 2 to 10, 2 to 5, 5 to 60, 5 to 50, 5 to 40, 5 to 30, 5 to 20, 5 to 10, 10 to 60, 10 to 50, 10 to 40, 10 to 30, 10 to 20, 20 to 60, 20 to 50, 20 to 40, 20 to 30, 30 to 60, 30 to 50, 30 to 40, 40 to 60, 40 to 50, or 50 to 60 days. In some embodiments, the treatment cycle is from 1 to 8 weeks (e.g., 1 to 7, 1 to 6, 1 to 5, 1 to 4, 1 to 3, 1 to 2, 2 to 8, 2 to 7, 2 to 6, 2 to 5, 2 to 4, 2 to 3, 3 to 8, 3 to 7, 3 to 6, 3 to 5, 3 to 4, 4 to 8, 4 to 7, 4 to 6, 4 to 5, 5 to 8, 5 to 7, 5 to 6, 6 to 8, 6 to 7, or 7 to 8 weeks). In some embodiments, the treatment cycle is or is about 1, 2, 3, 4, 5, 6, 7, or 8 weeks.

In some embodiments, the treatment regimen comprises from 1 to 5 treatment cycles, e.g., 1 to 4, 1 to 3, 1 to 2, 2 to 5, 2 to 4, 2 to 3, 3 to 5, 3 to 4, or 4 to 5 treatment cycles. In some embodiments, the treatment regimen comprises 1, 2, 3, 4, or 5 treatment cycles.

In some embodiments, the treatment cycles of a treatment regimen are the same (e.g., follow the same dosing and timing schedules). In some embodiments, the treatment cycles of a treatment regimen are different (e.g., follow different dosing and timing schedules).

In some embodiments, the treatment cycle comprises multiple administrations multispecific engager, e.g., a multispecific engager described herein and/or one or more doses of NK cells, e.g., NK cells described herein.

As part of a treatment cycle, the doses of the NK cells and of the multispecific engager are, in some cases, administered together (e.g., simultaneously or in succession during a single treatment day). In other cases, the NK cells and multispecific engager are administered separately (e.g., on different treatment days).

In some embodiments, the treatment cycle comprises treatment days spread evenly across the treatment cycle. For example, if the treatment cycle is 6 weeks long (or thereabouts), in some cases the treatment days occur every 1 week (or thereabouts).

In some embodiments, the treatment cycle comprises administration of the multispecific engager on each treatment day. In some embodiments, the treatment cycle comprises administration of the multispecific engager on a subset of the treatment days.

In some embodiments, the treatment cycle comprises administration of the NK cells on each treatment day. In some embodiments, the treatment cycle comprises administration of the NK cells on a subset of the treatment days. In some embodiments, NK cells are administered only during the first half of the treatment cycle.

In some embodiments, the treatment cycle further comprises administration of a cytokine, e.g., a cytokine described herein, e.g., IL-2. In some embodiments, the cytokine is administered together with the NK cells and/or multispecific engager (e.g., simultaneously with or in succession with the NK cells and/or multispecific engager). In other cases, the cytokine and is administered separately (e.g., on a different treatment day) from the NK cells and/or multispecific engagers. In some embodiments, the cytokine (e.g., IL-2) is administered only on days when NK cells are administered. In some embodiments, the cytokine (e.g., IL-2) is administered each day that NK cells are administered. In some embodiments, the cytokine (e.g., IL-2) is administered on some, but not all, days when NK cells are administered.

In some embodiments, the treatment cycle comprises administering doses of the multispecific engager spaced evenly (or thereabouts) over the treatment cycle (e.g., weekly over a six-week treatment cycle) and administering NK cells during the first half of the cycle (e.g., on the same treatment days as the multispecific engager during the first half of the treatment cycle), optionally along with IL-2 administration (e.g., as described above). Thus, in some cases, the treatment cycle comprises treatment days (e.g., three treatment days spaced evenly or thereabouts during the first three weeks of a six week treatment cycle, e.g., on days 1, 8, and 15) each comprising administration of the NK cells, the multispecific engager, and IL-2, followed by treatment days (e.g., three treatment days spaced evenly or thereabouts during the last three weeks of a six week treatment cycle, e.g., on days 22, 29, and 36) each comprising administration of only the multispecific engager. In some embodiments, the treatment regimen comprises repeating this treatment cycle is up to three times (e.g., 1, 2, or 3 times), and in some embodiments the treatment regimen comprises a treatment break (e.g., of 2 to 4 weeks or thereabouts) between each treatment cycle. In some embodiments, the first of these treatment cycles comprises lymphodepletion prior to the first treatment day (e.g., on days −3 and −4 or thereabouts for the first treatment cycle only). In some embodiments, each of these treatment cycles comprises lymphodepletion prior to the first treatment day (e.g., on days −3 and −4 or thereabouts for each treatment cycle).

In some embodiments, the treatment cycle further comprises lymphodepletion, e.g., as described herein. In some embodiments, the lymphodepletion is carried out prior to the administration of any doses of the NK cells or multispecific engagers during that cycle.

In some cases, the lymphodepletion for a treatment cycle is carried out from 1 to 5 days prior to administration of any doses of the NK cells or multispecific engagers, e.g., from 1 to 4, 1 to 3, 1 to 2, 2 to 5, 2 to 4, 2 to 3, 3 to 5, 3 to 4, or 4 to 5 days prior. In some cases, the lymphodepletion for a treatment cycle is carried out 1, 2, 3, 4, or 5 days prior to the administration of any doses of the NK cells or multispecific engagers for a treatment cycle.

In some cases, the treatment regimen comprises treatment cycles that each comprise lymphodepletion. In some cases, the treatment regimen comprises some treatment cycles that comprise lymphodepletion and some that do not. In some cases, the first treatment cycle of a treatment regimen comprises lymphodepletion, while the subsequent treatment cycles of the treatment regimen do not.

### F. Dosing

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a therapeutic compound (i.e., an effective dosage) depends on the therapeutic compounds selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

Dosage, toxicity and therapeutic efficacy of the therapeutic compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds may be within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

### VI. VARIANTS

In some embodiments, the molecule(s) or components thereof described herein, the fusion protein(s) or components thereof described herein, or the NK cell genotypes described herein, are at least 80%, e.g., at least 85%, 90%, 95%, 98%, or 100% identical to the amino acid sequence of an exemplary sequence (e.g., as provided herein), e.g., have differences at up to 1%, 2%, 5%, 10%, 15%, or 20% of the residues of the exemplary sequence replaced, e.g., with conservative mutations, e.g., including or in addition to the mutations described herein. In preferred embodiments, the variant retains desired activity of the parent.

To determine the percent identity of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is at least 80% of the length of the reference sequence, and in some embodiments is at least 90% or 100%. The nucleotides at corresponding amino acid positions or nucleotide positions are then compared. When a position in the first sequence is occupied by the same nucleotide as the corresponding position in the second sequence, then the molecules are identical at that position (as used herein nucleic acid “identity” is equivalent to nucleic acid “homology”). The percent identity between the two sequences is a function of the number of identical positions shared by the sequences, taking into account the number of gaps, and the length of each gap, which need to be introduced for optimal alignment of the two sequences.

Percent identity between a subject polypeptide or nucleic acid sequence (i.e. a query) and a second polypeptide or nucleic acid sequence (i.e. target) is determined in various ways that are within the skill in the art, for instance, using publicly available computer software such as Smith Waterman Alignment (Smith, T. F. and M. S. Waterman (1981) J Mol Biol 147:195-7): “BestFit” (Smith and Waterman, Advances in Applied Mathematics, 482-489 (1981)) as incorporated into GeneMatcher Plus™, Schwarz and Dayhof (1979) Atlas of Protein Sequence and Structure, Dayhof, M. O., Ed, pp 353-358: BLAST program (Basic Local Alignment Search Tool: (Altschul, S. F., W. Gish, et al. (1990) J Mol Biol 215:403-10), BLAST-2, BLAST-P, BLAST-N, BLAST-X, WU-BLAST-2, ALIGN, ALIGN-2, CLUSTAL, or Megalign (DNASTAR) software. In addition, those skilled in the art can determine appropriate parameters for measuring alignment, including any algorithms needed to achieve maximal alignment over the length of the sequences being compared. In general, for target proteins or nucleic acids, the length of comparison can be any length, up to and including full length of the target (e.g., 5%, 10%, 20%, 30%, 40%, 50%, 60%, 70%, 80%, 90%, 95%, or 100%). For the purposes of the present disclosure, percent identity is relative to the full length of the query sequence.

For purposes of the present disclosure, the comparison of sequences and determination of percent identity between two sequences can be accomplished using a Blossum 62 scoring matrix with a gap penalty of 12, a gap extend penalty of 4, and a frameshift gap penalty of 5.

Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

### VII. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein.

The term “in vivo” is used to describe an event that takes place in a subject's body.

The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

As used herein, the term “about” a number refers to that number plus or minus 10% of that number. The term “about” a range refers to that range minus 10% of its lowest value and plus 10% of its greatest value.

As used herein, the term “buffer solution” refers to an aqueous solution consisting of a mixture of a weak acid and its conjugate base, or vice versa.

As used herein, the term “cell culture medium” refers to a mixture for growth and proliferation of cells in vitro, which contains essential elements for growth and proliferation of cells such as sugars, amino acids, various nutrients, inorganic substances, etc.

A buffer solution, as used herein, is not a cell culture medium.

As used herein, the term “bioreactor” refers to a culture apparatus capable of continuously controlling a series of conditions that affect cell culture, such as dissolved oxygen concentration, dissolved carbon dioxide concentration, pH, and temperature.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Some vectors are suitable for delivering the nucleic acid molecule(s) or polynucleotide(s) of the present application. Certain vectors are capable of directing the expression of nucleic acids to which they are operatively linked. Such vectors are referred to herein as expression vectors.

The term “operably linked” refers to two or more nucleic acid sequence or polypeptide elements that are usually physically linked and are in a functional relationship with each other. For instance, a promoter is operably linked to a coding sequence if the promoter is able to initiate or regulate the transcription or expression of a coding sequence, in which case, the coding sequence should be understood as being “under the control of” the promoter.

The terms “host cell,” “host cell line,” and “host cell culture” are used interchangeably and refer to cells into which exogenous nucleic acid has been introduced, including the progeny of such cells. Host cells include “engineered cells,” “transformants,” and “transformed cells,” which include the primary engineered (e.g., transformed) cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

As appropriate, the host cells can be stably or transiently transfected with a polynucleotide encoding a fusion protein, as described herein.

The section headings used herein are for organizational purposes only and are not to be construed as limiting the subject matter described.

### VIII. EXAMPLES

The following examples are included for illustrative purposes only and are not intended to limit the scope of the invention.

### Example 1: Off-the-Shelf NK Cell Therapy Platform

One example of a method by which NK cells were expanded and stimulated is shown in FIG. 1. A single unit of FDA-licensed, frozen cord blood that has a high affinity variant of the receptor CD16 (the 158 V/V variant, see. e.g., Koene et al., “FcγRIIIa-158V/F Polymorphism Influences the Binding of IgG by Natural Killer Cell FcgammaRIIIa, Independently of the FcgammaRIIIa-48L/R/H Phenotype,” Blood 90:1109-14 (1997).) and the KIR-B genotype (KIR B allele of the KIR receptor family, see. e.g., Hsu et al., “The Killer Cell Immunoglobulin-Like Receptor (KIR) Genomic Region: Gene-Order, Haplotypes and Allelic Polymorphism,” Immunological Review: 190:40-52 (2002); and Pyo et al., “Different Patterns of Evolution in the Centromeric and Telomeric Regions of Group A and B Haplotypes of the Human Killer Cell Ig-like Receptor Locus,” PLoS One 5: e15115 (2010)) was selected as the source of NK cells.

The cord blood unit was thawed and the freezing medium was removed via centrifugation. The cell preparation was then depleted of T cells using the QuadroMACS Cell Selection System (Miltenyi) and CD3 (T cell) MicroBeads. A population of 6×10⁸ total nucleated cells (TNC) were labelled with the MicroBeads and separated using the QuadroMACS device and buffer. Following depletion of T cells, the remaining cells, which were predominantly monocytes and NK cells, were washed and collected in antibiotic-free medium (CellgroSCGM). The cell preparation was then evaluated for total nucleated cell count, viability, and % CD3+ cells. As shown in FIG. 1, the cord blood NK cells were CD3 depleted.

The CD3-cell preparation was inoculated into a gas permeable cell expansion bag containing growth medium. The cells were co-cultured with replication incompetent engineered HuT-78 (eHUT-78) feeder cells to enhance expansion for master cell bank (MCB) production. The CellgroSCGM growth media was initially supplemented with 10 ng/ml of anti-CD3 antibody (OKT3), human plasma, glutamine, and IL-2.

As shown in FIG. 1, the NK cells are optionally engineered, e.g., to introduce CARs into the NK cells, e.g., with a lentiviral vector, during one of the co-culturing steps.

The cells were incubated as a static culture for 12-16 days at 37° C. in a 5% CO₂ balanced air environment, with additional exchanges of media occurring every 2 to 4 days. After the culture expanded more than 100-fold, the cultured cells were harvested and then suspended in freezing medium and filled into cryobags. In this example, 80 bags or vials at 10⁸ cells per bag or vial were produced during the co-culture. The cryobags were frozen using a controlled rate freezer and stored in vapor phase liquid nitrogen (LN₂) tanks below-150° C. These cryopreserved NK cells derived from the FDA-licensed cord blood unit served as the master cell bank (MCB).

To produce the drug product, a bag of frozen cells from the MCB was thawed and the freezing medium was removed. The thawed cells were inoculated into a disposable culture bag and co-cultured with feeder cells, e.g., eHUT78 feeder cells to produce the drug product. In this example, the cells are cultured in a 50 L bioreactor to produce thousands of lots of the drug product per unit of cord blood (e.g., 4,000-8,000 cryovials at 10⁹ cells/vial), which are mixed with a cryopreservation composition and frozen in a plurality of storage vessels such as cryovials. The drug product is an off-the-shelf infusion ready product that can be used for direct infusion. Each lot of the drug product can be used to infuse hundreds to thousands of patients (e.g., 100-1,000 patients, e.g. with a target dose of 4×10⁹ cells).

### Example 2: Feeder Cell Expansion

As one example, suitable feeder cells, e.g., eHut-78 cells, were thawed from a frozen stock and expanded and cultured in a 125 mL flask in growth medium comprising RPMI1640 (Life Technologies) 89% v/v, inactivated fetal bovine serum (FBS) (Life Technologies) (10% v/v), and glutamine (hyclone) (2 mM) at or at about 37° C. and at or at about 3-7% CO₂ for or for about 18-24 days. The cells were split every 2-3 days into 125 ml-2L flasks. The cells were harvested by centrifugation and gamma irradiated. The harvested and irradiated cells were mixed with a cryopreservation medium (Cryostor CS10) in 2 mL cryovials and frozen in a controlled rate freezer, with a decrease in temperature of about 15° C. every 5 minutes to a final temperature of or of about −90° C., after which they were transferred to a liquid nitrogen tank or freezer to a final temperature of or of about −150° C.

After freezing, cell viability was greater than or equal to 70% of the original number of cells (here, at least 1.0×10⁸ viable cells/mL), and 85% or more of the cells expressed mTNF-α, 85% or more of the cells expressed mbIL-21+, and 85% or more of the cells expressed 4-1BBL.

### Example 3: NK Cell Expansion and Stimulation

As one example, suitable NK cells can be prepared as follows using HuT-78 cells transduced to express 4-1BBL, membrane bound IL-21 and mutant TNFalpha (“eHut-78P cells”) as feeder cells. The feeder cells are suspended in 1% (v/v) CellGro medium and are irradiated with 20,000 cGy in a gamma-ray irradiator. Seed cells (e.g., CD3-depleted PBMC or CD3-depleted cord blood cells) are grown on the feeder cells in CellGro medium containing human plasma, glutamine, IL-2, and OKT-3 in static culture at 37° C. The cells are split every 2-4 days. The total culture time was 19 days. The NK cells are harvested by centrifugation and cryopreserved. Thawed NK are administered to patients in infusion medium consisting of: Phosphate Buffered Saline (PBS 1×, FujiFilm Irvine) (50% v/v), albumin (human) (20% v/v of OctaPharma albumin solution containing: 200 g/L protein, of which ≥96% is human albumin, 130-160 mmol sodium: ≤2 mmol potassium, 0.064-0.096 mmol/g protein N-acetyl-DL-tryptophan, 0.064-0.096 mmol/g protein, caprylic acid, ad. 1000 ml water), Dextran 40 in Dextrose (25% v/v of Hospira Dextran 40 in Dextrose Injection, USP containing: 10 g/100 mL Dextran 40 and 5 g/100 mL dextrose hydrous in water) and dimethyl sulfoxide (DMSO) (5% v/v of Avantor DMSL solution with a density of 1.101 g/cm³ at 20° C.).

In some case, the seed cells are CD3-depleted cord blood cells. A cell fraction can be depleted of CD3 cells by immunomagnetic selection, for example, using a CliniMACS T cell depletion set ((LS Depletion set (162-01) Miltenyi Biotec).

Preferably, the cord blood seed cells are selected to express CD16 having the V/V polymorphism at F158 (Fc gamma RIIIa-158 V/V genotype) (Musolino et al. 2008 J Clin Oncol 26:1789). Preferably, the cord blood seed cells are KIR-B haplotype.

### Example 4: Cord Blood as an NK Cell Source

NK cells make up five to 15% of peripheral blood lymphocytes. Traditionally, peripheral blood has been used as the source for NK cells for therapeutic use. However, as shown herein, NK cells derived from cord blood have a nearly ten-fold greater potential for expansion in the culture systems described herein than those derived from peripheral blood, without premature exhaustion or senescence of the cells. The expression of receptors of interest on the surface of NK cells, such as those involved in the activation of NK cells on engagement of tumor cells, was seen to be more consistent donor-to-donor for cord blood NKs than peripheral-blood NK cells. The use of the manufacturing process described herein consistently activated the NK cells in cord blood in a donor-independent manner, resulting in a highly scaled, active and consistent NK cell product.

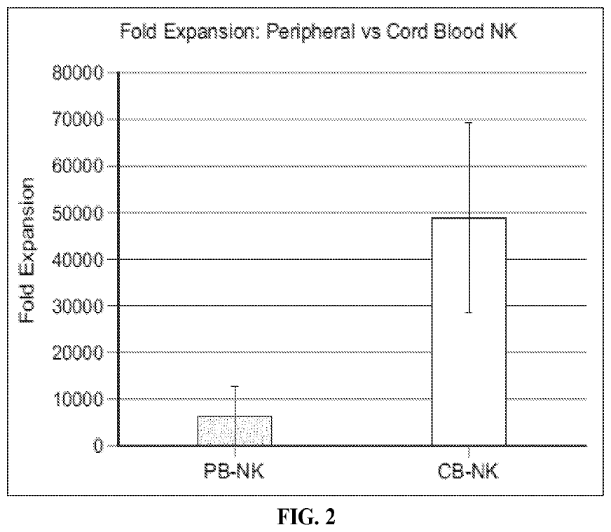

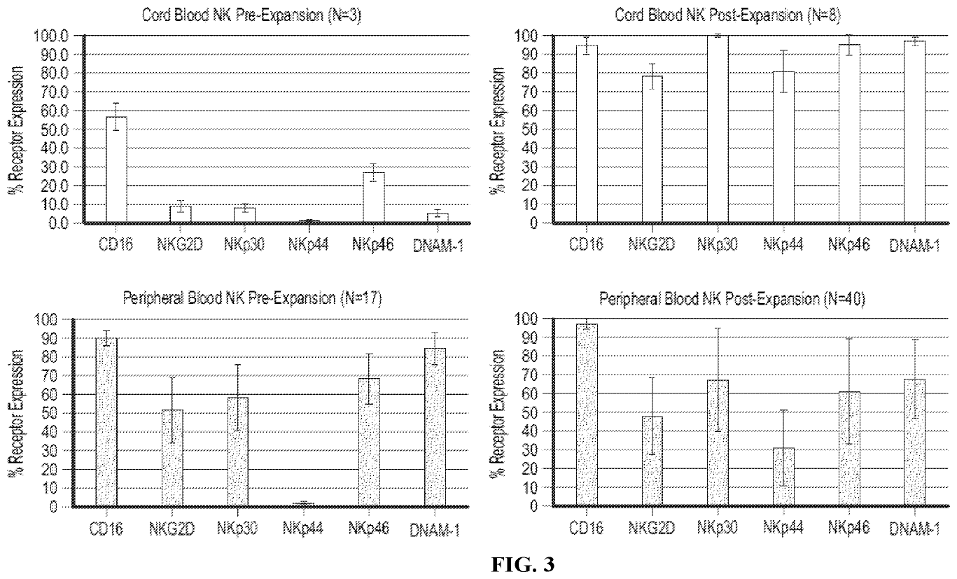

As shown in FIG. 2, cord blood-derived NK cells (CB-NK) have an approximately ten-fold greater ability to expand in culture than peripheral blood-derived NK cells (PB-NK) in preclinical studies. As shown in FIG. 3, expression of tumor-engaging NK activating immune receptors was higher and more consistent in cord blood-derived drug product compared to that generated from peripheral blood.

### Example 5: Expanded and Stimulated NK-Cell Phenotype

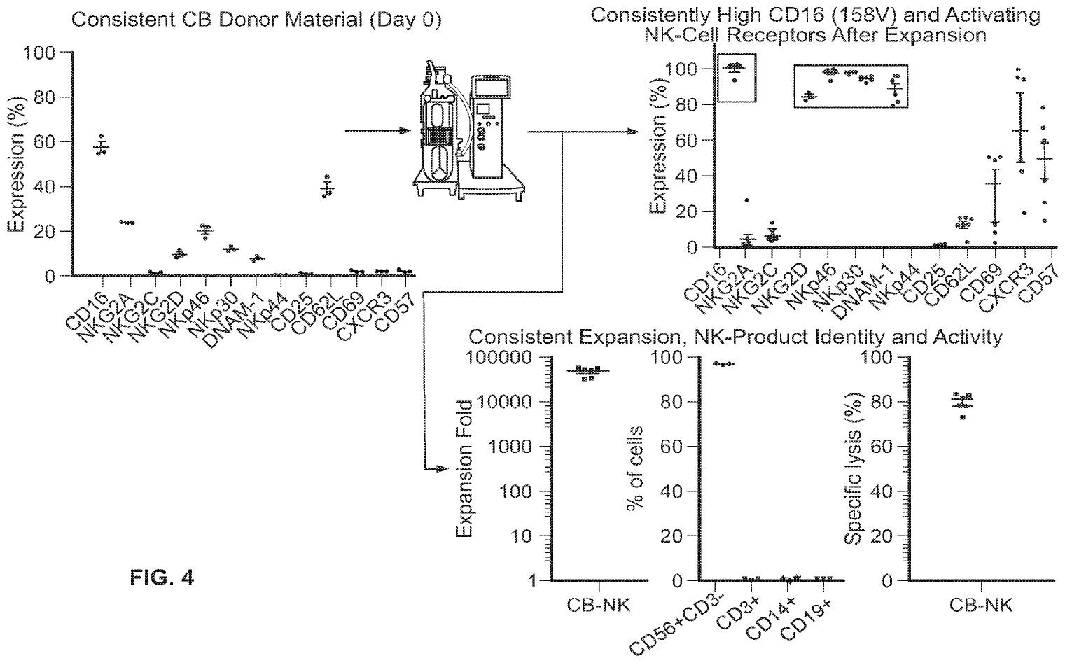

In one example, NK cells from a cord blood unit are expanded and stimulated with eHut-78 cells, according to the expansion and stimulation process described in Example 1. As shown in FIG. 4, the resulting expanded and stimulated population of NK cells have consistently high CD16 (158V) and activating NK-cell receptor expression.

### Example 6: AB-101

AB-101 is a universal, off-the-shelf, cryopreserved allogeneic cord blood derived NK cell therapy product comprising ex vivo expanded and activated effector cells designed to enhance ADCC anti-tumor responses in patients, e.g., patients treated with monoclonal antibodies or NK cell engagers. AB-101 is comprised of cord blood derived mononuclear cells (CBMCs) enriched for NK cells by depletion of T lymphocytes, and co-cultured with an engineered, replication incompetent T cell feeder line supplemented with IL-2 and anti-CD3 antibody (OKT3).

AB-101 is an allogeneic NK-cell product derived from FDA licensed cord blood, specifically designed to treat hematological and solid tumors in combination with therapeutic monoclonal antibodies (mAbs). The AB-101 manufacturing process leads to an NK cell product with the following attributes:
Consistent NK cell profile. High surface receptor expression of antibody engaging CD16 and tumor antigen-engaging/activating receptors such as NKG2D, NKp46, Nkp30 and NKp44.
KIR-B-haplotype. KIR-B haplotype has been associated with improved clinical outcomes in the haploidentical transplant setting and greater therapeutic potential in the allogeneic setting
CD16 F158V polymorphism. The higher-affinity CD16 F158V variant binding to mAb Fc-domain is seen to facilitate enhanced antibody dependent cellular cytotoxicity (ADCC).
Unmodified NK cells. No genetic enhancement or gene editing is required for, or is a part of, the AB-101 drug product.

The components and composition of AB-101 are listed in Table 6. AB-101 is comprised of NK cells (CD16+, CD56+) expressing the natural cytotoxicity receptors NKp30 and NKp46 indicative of mature NK cells. AB-101 contains negligible T cells, B cells and macrophages (≤0.2% CD3⁺, ≤1.0% CD19⁺, ≤1.0% CD14⁺). Residual eHuT-78P feeder cells used in the culturing of AB-101 are ≤0.2% of the drug product.

TABLE 6
Components and Compositions of AB-101

| | Solution | | | | Quantity per Unit |
|---|---|---|---|---|---|
| Component Solution | Composition | Conc | Conc | | (11 mL fill) |
| AB-101 drug | Approximately | 50% v/v | 0.5 | mL/mL | 5.5 mL |
| substance (ex vivo- | 1.1 × 10⁹ | | | | (0.9 × 10⁹-1.3 × 10⁹ |
| expanded allogeneic | viable cells | | | | viable cells per vial in |
| natural killer cells) | | | | | 5.27-6.23 mL of PBS) |
| PBS | 100% Phosphate | | | | |
| | Buffered | | | | |
| | Saline (PBS) | | | | |
| Albumin Solution | 200 g/L albumin | 20% v/v | 40 mg/mL albumin | | 2.2 mL |
| | in water | | | | (1.98-2.42 mL) |
| Dextran 40 Solution | 100 g/L Dextran | 25% v/v | 25 mg/mL Dextran | | 2.75 mL |
| | 40; and | | 40; | | (2.475-3.025 mL) |
| | 50 g/L glucose | | 12.5 mg/mL glucose | | |
| | in water | | | | |
| DMSO | 100% DMSO | 5% v/v | 55 | mg/mL | 0.55 mL |
| | (1,100 g/L) | | | | (0.495-0.605 mL) |

Initial stability studies indicate that AB-101 is stable for up to six months in the vapor phase of liquid nitrogen. Long-term stability studies to assess product stability beyond six months are ongoing, and the most current stability information will be captured on the certificate of analysis.

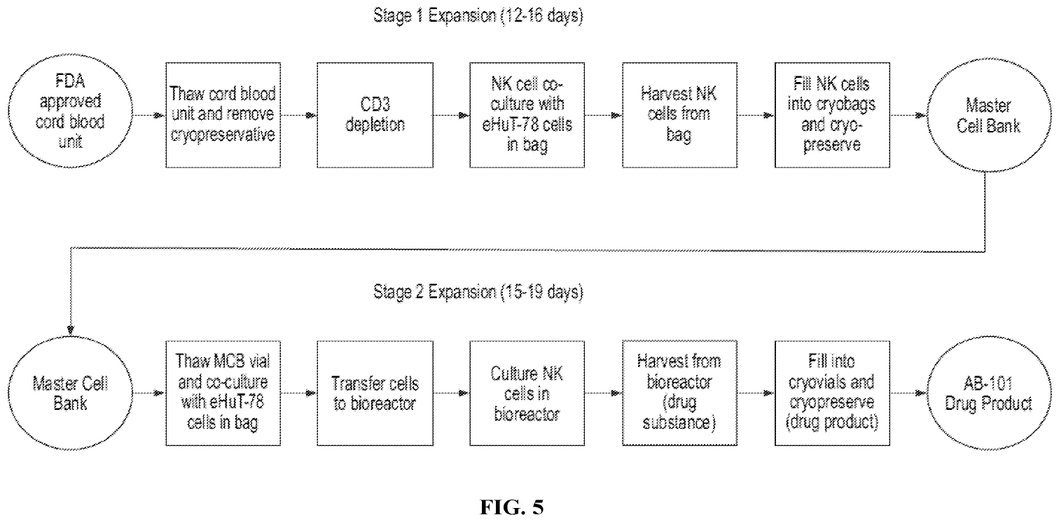

The manufacture of the AB-101 drug product is comprised of the following key steps (FIG. 5):
Thaw of the FDA licensed cord blood unit (Hemacord, BLA 125937).
Removal of cyro-preservation medium from the cord blood unit (CBU)
CD3 depletion using FDA cleared Vario MACS Cell Selection System (Miltenyi)
Expansion and co-culture in bags with an engineered feeder cell line (eHuT-78 cells)
Testing and cryopreservation of the AB-101 master cell bank (approximately 200 bags)
Thaw (single bag), expand and co-culture with engineered HuT-78 cells
Further expansion in bioreactor
Harvest and fill (1×10⁹ NK cells per vial)
Cryopreservation of the AB-101 drug product (approximately 150 vials)
Extensive characterization to determine consistency, purity, potency and safety.

As shown in Table 7, this manufacturing process reproducibly generates very large quantities of highly pure and active AB-101 drug product NK cells. Data points represent products generated from three independent cord blood units.

TABLE 7
AB-101 Product Characterization

| | | Acceptance | Engineering Batches | | | Clinical Batches | | | |
|---|---|---|---|---|---|---|---|---|---|
| Test Attribute | | Criterion | 1 | 2 | 3 | 1 | 2 | 3 | 4 |
| Cell Count (cells/vial) | | 0.9-1.3 × | 1.3 × | 1.1 × | 1.0 × | 1.3 × | 1.2 × | 1.2 × | 1.0 × |
| | | 10⁹ | 10⁹ | 10⁹ | 10⁹ | 10⁹ | 10⁹ | 10⁹ | 10⁹ |
| Cell Viability | | ≥70% | 96% | 95% | 94% | 93% | 94% | 94% | 94% |
| Endotoxin (EU/mL) | | ≤5 | ≤1 | ≤1 | ≤1 | ≤1 | ≤1 | ≤1 | ≤1 |
| Identity | CD3−, | ≥85% | 99.16% | 99.79% | 99.43% | 99.53% | 98.40% | 97.87% | 98.54% |
| | CD56+ % | | | | | | | | |
| | CD56+, | ≥70% | 94.42% | 94.20% | 99.04% | 93.24% | 91.72% | 95.22% | 90.21% |
| | CD16+ % | | | | | | | | |
| Purity | CD3+ % | (CD3+) ≤ | ≤0.00% | 0.00% | 0.00% | 0.06% | 0.00% | 0.00% | 0.02% |
| | | 0.20% | | | | | | | |
| | CD14+ % | (CD14+) ≤ | ≤0.02% | 0.00% | 0.00% | 0.02% | 0.03% | 0.01% | 0.10% |
| | | 1.00% | | | | | | | |
| | CD19+ % | (CD19+) ≤ | ≤0.01% | 0.01% | 0.00% | 0.00% | 0.00% | 0.05% | 0.05% |
| | | 1.00% | | | | | | | |

| Potency | ≥50% | 69.00% | 60.20% | 64.10% | 64.50% | 67.10% | 54.80% | 67.40% |
|---|---|---|---|---|---|---|---|---|
| | killing at 4 | | | | | | | |
| | hours | | | | | | | |

### Identity (CD3−, CD56+)

The frequency of CD3−, CD56+ cells are used to assess the identity of AB-101 Drug Product. A sample of AB-101 Drug Product is thawed and resuspended in a staining buffer. The resuspended sample is added to fluorochrome-labeled antibodies that bind to CD3+ and CD56+ surface antigens. Flow cytometry is used to determine percent populations of CD3−, CD56+ as a measure of product identity.

### Identity (CD56+, CD16+)

The frequency of CD56+, CD16+ cells are used to assess the identity of AB-101 Drug Product. A sample of AB-101 Drug Product is thawed and resuspended in a staining buffer. The resuspended sample is added to fluorochrome-labeled antibodies that bind to CD56+ and CD16+ surface antigens. Flow cytometry is used to determine percent populations of CD56+, CD16+ as a measure of product identity.

### Purity (CD3+)

Measurement of CD3+ expressing cells are used to assess the purity of AB-101 Drug Product. Flow cytometry method is used to determine the purity of the drug product for CD3+ expressing cells. The percent population of CD3+ cells is used as a measure of product purity.

### Purity (CD14+)

Measurement of CD14+ expressing cells are used to assess the purity of AB-101 Drug Product. Flow cytometry method is used to determine the purity of the drug product for CD14+ expressing cells. The percent population of CD14+ cells is used as a measure of product purity.

### Purity (CD19+)

Measurement of CD19+ expressing cells are used to assess the purity of AB-101 Drug Product. Flow cytometry method is used to determine the purity of the drug product for CD19+ expressing cells. The percent population of CD19+ cells is used as a measure of product purity.

Purity: Residual eHuT-78P (Residual eHuT-78P Cells)

Residual eHuT-78P cells in AB-101 drug product are measured by flow cytometry (FACS). FACS is used detect residual eHuT-78 in AB-101 DP by quantifying the live CD3+4-1BBLhigh+ eHuT-78P. The FACS gating strategy (See FIG. 1), which sequentially gates, singlet, 7-AAD and CD3+4-1BBL+, was used because eHuT-78 is derived from a HuT-78 cell line that expresses CD3 as cutaneous T lymphocyte. The HuT-78 cell line was transduced by 4-1BB ligand (4-1BBL), membrane tumor necrosis factor-a (mTNF-a) and membrane bound IL-21 (mbIL-21). An eHuT-78 single cell that highly expresses the three genes was selected, and research, master and working cell banks were successively established. Among the three genes, 4-1BBL was utilized for the FACS gating strategy because it showed the highest expression in AB-101 cell bank and final drug product.

### Potency (Cytotoxicity at 10:1 AB-101 DP Cells to K562 Cells)

Potency of AB-101 Drug Product is determined by evaluating capacity for cellular cytotoxicity against K562 tumor cells. Cytotoxicity of the drug product will be assessed by fluorometric assay. K562 tumor cells are stained with 30 μM calcein-AM (Molecular probe) for 1 hour at 37° C. A sample of the drug product and the labeled tumor cells are co-cultured in a 96-well plate in triplicate at 37° C. and 5% CO2 for 4 hours with light protection. RPMI1640 medium containing 10% FBS or 2% triton-X100 was added to the targets to provide spontaneous and maximum release. RPMI1640 medium containing 10% FBS or 2% triton-X100 is added to each well to determine background fluorescence. The measurement of fluorescence is conducted at excitation of 485 nm and emission 535 nm with a florescent reader. The percent specific cytotoxicity is calculated by the following formula.

$$\% Specific cytotoxicity = 100 \times \frac{\% specific death - \% spontaneous death}{100 - \% spontaneous death}$$

### Potency (Cytotoxicity at 10:1 AB-101 DP Cells to Ramos Cells)

Potency of AB-101 Drug Product is also determined by evaluating the capacity for cellular cytotoxicity against Ramos tumor cells using the same method and calculation described above. The specification for this testing is being determined.

### Example 7: AB-101 Phenotypic Characterization

The purity as well as expression of antibody-engaging CD16 and activating, inhibitory and chemokine receptors of multiple batches of AB-101 were measured via flow cytometry.

AB-101 purity was measured using cell surface markers: AB-101 batches were seen to comprise >99% CD3-CD56+NK cells and <0.1% CD3+, CD14+ and CD19+ cells. CD16 expression of AB-101 was measured. 95.11±2.51% of AB-101 cells were CD16+ with mean and median MFI of CD16 15311±6186 and 13097±5592 respectively. NK cells are known to express various NK specific activating and inhibitory receptors. For the various AB-101 batches that were tested, >80% of cells expressed CD16, NKG2A, NKG2D, CD94, NKp30, 2B4, Tim-3, CD44, 40˜70% of cells expressed NKp44, NKp46, DNAM-1, approximately 30% of cells expressed CD161 and CD96, 15% of cells expressed CXCR3, and less than 5% of cells expressed other activating inhibitory receptors.

Two GMP batches of AB-101 were included in the study to assess the phenotypic characteristics of NK cells at three different stages of the manufacturing process: Cord blood cells post CD3+ cell depletion: master cell bank (MCB) as intermediate, and AB-101 final drug product (DP). The CD3 depleted cells, MCB and DP, each were measured for purity and NK cell receptors. Based on the results, it was seen that NK cells initially derived from CB showed immature NK phenotypes. The NK phenotype matured during the manufacturing process. At the MCB stage, more than 90% of cells already expressed the phenotypic characteristic seen in matured NK cells, and markers of other cell types were <0.1%. The expression level for most of the NK cell-specific receptors increased throughout the manufacturing process from CD3 depleted cells, to MCB and finally DP

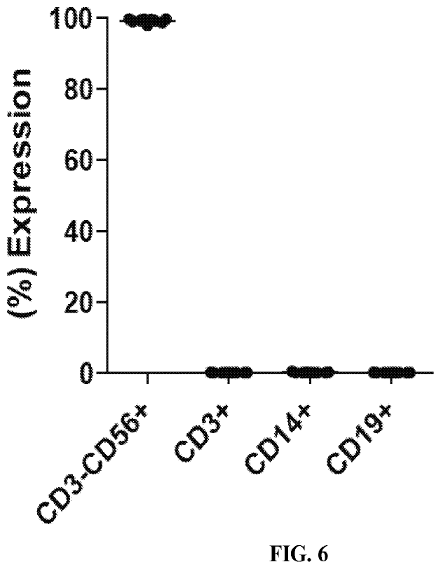

The purity of AB-101 is represented as CD3-CD56+ cells for NK cells, CD3+ cells for T-cells, CD14+ cells for monocytes and CD19+ cells for B-cells. Total 9 batches of AB-101 were measured for the purity. The results showed 99.27±0.59% (mean±SD) for CD3-CD56+ cells, 0.02±0.03% for CD3+ cells, 0.10+0.12% for CD14+ cells, and 0.02=0.04% for CD19+ cells (FIG. 6). Therefore, it was confirmed that AB-101 is composed of high-purity of NK cells, and the other types of cells as impurities were rarely present.

### Comparison of Purity of CD3 Depleted Cells, MCB, and DP Manufactured in GMP Conditions.

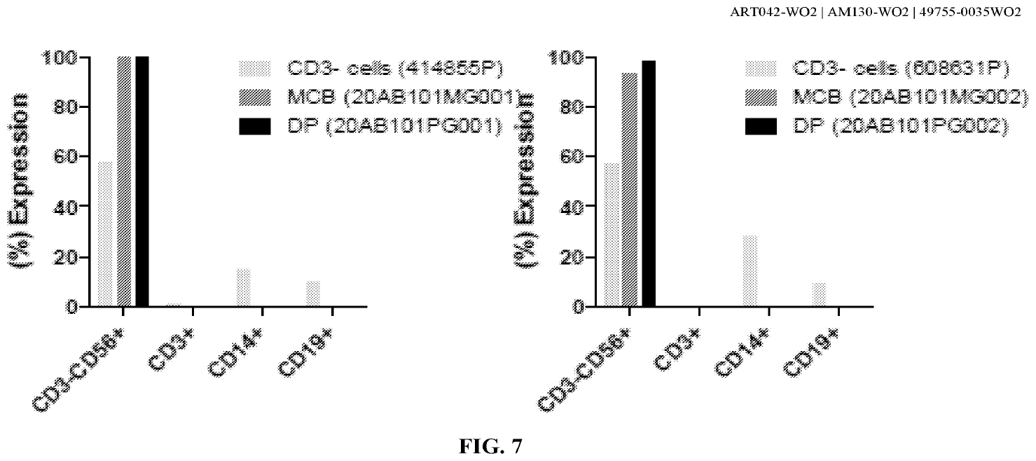

Two GMP batches of AB-101 were utilized to assess the purity of AB-101 starting material (CD3 depleted cells), intermediate (master cell bank, MCB), and final drug product (DP). 50˜60% of cells in CD3 depleted cell fraction were NK cells, and these percentages increased to more than 90% in MCB and DP. CD14+ cells and CD19+ cells were representative of 20˜30% of CD3 depleted cell fraction, and these cell percentages decreased to less than 0.1% in MCB and DP indicative of purity of AB-101 MCB and AB-101 final drug products (FIG. 7, Table 8).

TABLE 8
Cell Purity

| | GMP batch #1 | | | GMP batch #2 | | |
|---|---|---|---|---|---|---|
| | CD3− | | | CD3− | | |
| | cells | MCB | DP | cells | MCB | DP |
| Marker | (414855P) | (20AB101MG001) | (20AB101PG001) | (608631P) | (20AB101MG002) | (20AB101PG002) |
| CD3− | 58.0 | 99.43 | 99.80 | 56.70 | 93.14 | 97.98 |
| CD56+ | | | | | | |
| (%) | | | | | | |
| CD3+ | 0.79 | 0.05 | 0.01 | 0.21 | 0.03 | 0.02 |
| (%) | | | | | | |
| CD14+ | 15.01 | 0.02 | 0.01 | 28.00 | 0.03 | 0.02 |
| (%) | | | | | | |
| CD19+ | 9.83 | 0.01 | 0.00 | 9.17 | 0.00 | 0.00 |
| (%) | | | | | | |

### Comparison of NK Cell Receptors of CD3 Depleted Cells, MCB, and DP Manufactured in GMP Conditions

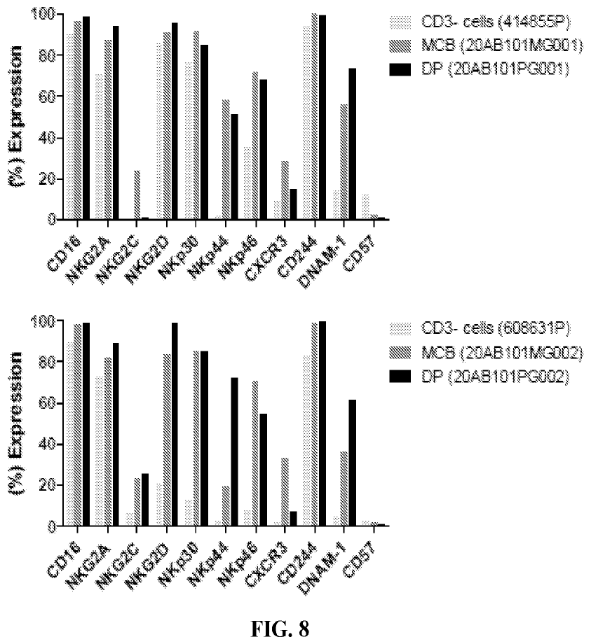

Two GMP batches of AB-101 were also utilized to assess the expression of various NK cell receptors on AB-101 starting material (CD3 depleted cells), intermediate (master cell bank, MCB), and final drug product (DP). It was observed that several NK cell and activating receptors such as CD16, NKG2D, NKG2C, NKp30, NKp44, NKp46 and DNAM-1 were expressed in higher levels by MCB, final drug product when compared to AB-101 starting material (CD3 depleted cells). The CD57 expression was lower in MCB and final drug product when compared to AB-101 starting material (CD3 depleted cells) (FIG. 8, Table 9). Overall, data shows an increase in expression of NK cell activating receptors in MCB and DP indicative of AB-101 being effective against tumors.

TABLE 9
Cell Receptor Expression

| | GMP batch #1 | | | GMP batch #2 | | |
|---|---|---|---|---|---|---|
| | CD3− | | | CD3− | | |
| | cells | MCB | DP | cells | MCB | DP |
| Marker | (414855P) | (20AB101MG001) | (20AB101PG001) | (608631P) | (20AB101MG002) | (20AB101PG002) |
| Cd16 | 90.27 | 96.45 | 98.50 | 89.27 | 97.70 | 98.30 |
| NKG2A | 69.99 | 87.05 | 93.70 | 72.94 | 81.92 | 88.43 |
| NKG2C | 0.26 | 23.87 | 1.11 | 6.32 | 22.91 | 25.04 |
| NKG2D | 85.52 | 91.13 | 95.17 | 20.70 | 83.16 | 98.77 |
| NKp30 | 76.29 | 91.55 | 94.64 | 12.61 | 85.19 | 85.22 |
| NKp44 | 1.29 | 58.27 | 51.14 | 2.48 | 19.15 | 72.03 |
| NKp46 | 35.12 | 71.83 | 67.77 | 7.64 | 70.54 | 54.46 |
| CXCR3 | 9.10 | 28.39 | 14.40 | 1.79 | 33.13 | 7.01 |
| 2B4 | 93.66 | 99.75 | 99.20 | 82.63 | 98.29 | 99.46 |
| DNAM-1 | 13.94 | 55.64 | 73.07 | 5.12 | 36.24 | 61.13 |
| CD57 | 12.24 | 1.92 | 0.65 | 2.63 | 1.63 | 0.74 |

### Conclusion

The use of surface marker analysis supported the identity and purity and batch-to-batch consistency of the AB-101 product. Further, extensive assessment of NK-specific activating and inhibitory cell surface markers established the consistent profile of the AB-101 product post manufacturing expansion process. It is known that CB derived NK cells have immature phenotype such as high expression of NKG2A and low expression of NKG2C, CD62L, CD57, IL-2R, CD16, DNAM-1 comparing to peripheral blood (PB) derived NK cells, and it is also known that CB derived NK cells with the immature phenotypes exhibit low cytotoxicity against tumor cells. Data from this report shows that AB-101, an allogeneic cord blood (CB) derived NK cell product, expresses high levels of major activating receptors indicative of potential higher cytotoxicity against tumor cells.

### Example 8: AB-101 Pharmacokinetics and Biodistribution

The NOD scid gamma (NSG) mouse model was used to determine the biodistribution and pharmacokinetics (PK) of AB-101. Vehicle (PBS, Dextran, Albumin (human) DMSO) and AB-101 cells (0.5×10⁷ cells/mouse, 2×10⁷ cells/mouse) were administered intravenously (0.25 mL/mouse) for a total of 8 doses. Animals in vehicle and AB-101 groups were sacrificed at timepoints 4 hr, 1, 3, 7, 14 and 78 days (n=3 male mice, n=3 female mice per timepoint) post last dose infusion.

AB-101 was detected predominantly in highly perfused tissues (lungs, spleen, heart and liver) and at the site of injection starting at 4 hrs after administration, until 3 days after administration of final dose of AB-101 (day 53). At 7 days after administration of final dose (day 57) AB-101 was detected in lung (3 out of 6 samples), spleen (5 out of 6 samples) and injection site (5 out of 6 samples). At 14 days and 28 days after administration of final dose (day 64 and day 78 respectively), AB-101 was detected in two and one injection site samples, respectively. The sporadic incidence and low concentrations observed from the injection site samples at day 64 and day 78 would not be indicative of systemic persistence of the AB-101 test article.

The results from the biodistribution studies indicate that the distribution of AB-101 in vivo is consistent with the intravenous route of administration and that the cells lack long-term persistence potential with tissue clearance after 7 days post-administration and no evidence of permanent engraftment.

### Example 9: AB-101 Toxicology

Nonclinical toxicity of AB-101 was assessed in a GLP study of NSG mice. The study was designed to evaluate the acute and delayed toxicity profile of AB-101. Two dose levels of AB-101, 0.5×10⁷ and 2×10⁷ cells/animal, were tested in the study. The proposed test dose range was designed to deliver a greater exposure of the product than the planned highest equivalent human dose to be given in a first-in-human study (4×10⁹ cells per dose). Based on allometric scaling (Nair 2016), 0.5×10⁷ cells/mouse corresponded to 14×10⁹ cells/human, and 2×10⁷ cells/mouse corresponded to 56×10⁹ cells/human, assuming a patient weighing 70 kg. AB-101 was administered intravenously once weekly for 8 weeks via the tail vein. Acute toxicity of AB-101 was evaluated 3 days after the eighth dose (i.e., last dose). Delayed toxicity was evaluated at the end of the 28 days recovery period after the eighth dose. Viability, body weight, clinical observations and palpations were recorded for each animal during the in-life portion of the study. Gross necropsy and sample collection for hematology, clinical chemistry and histopathology analysis were performed at the time of euthanasia for all animals.

Each group contained 20 animals in total, with 10 of each gender, to evaluate findings in both sexes and for powered statistical analysis. A vehicle treated control group was included for comparison to the AB-101 treated groups. To minimize treatment bias, animals were assigned to dose groups based on computer-generated (weight-ordered) randomization procedures, with male and females randomized separately. The study adhered to GLP guidelines, including those for data reporting.

No mortality and no adverse clinical observations were recorded related to administration of AB-101 at any of the evaluated dose levels. All minor clinical observations that were noted are common findings in mice and were not considered related to AB-101 administration. Body and organ weight changes were comparable among dose groups and different days of post-treatment assessment (Day 53 for acute toxicity groups and Day 78 for delayed toxicity groups). There were no AB-101-related changes in hematology and clinical chemistry parameters or gross necropsy findings noted in animals at euthanasia in either the acute or delayed toxicity groups. All fluctuations among individual and mean clinical chemistry values, regardless of statistical significance, were considered sporadic, consistent with biologic and procedure-related variation, and/or negligible in magnitude, and therefore deemed not related to AB-101 administration. There were no AB-101-related microscopic findings. In conclusion, results from the GLP toxicity study indicate that AB-101 is well tolerated in NSG mice with repeated dosing of up to 2×10⁷ cells/dose/animal.

### Example 10: Cryopreservation of NK Cells

AB-101 cells were prepared by the process shown in FIG. 5. At the end of the culture period the cells were harvested through the use of a Sartorius kSep® 400 Single-Use Automated Centrifugation System at Relative Centrifugal Field (RCF): 800-1200 g with a flow rate at 60 to 120 mL/min, and washed two times with Phosphate Buffer Solution (PBS). After washing, the AB-101 cells were formulated with: (1) Albumin (human); (2) Dextran 40; (3) DMSO and (4) PBS to a target concentration of 1× 10⁸ cells/mL (exemplary cryopreservation composition #1, Table 4). The formulated suspension was then filled at a target volume of 11 mL into 10 mL AT-Closed Vial®. Filled vials were inspected, labeled and cryopreserved in a controlled rate freezer at ≤−135° C.

Stability studies were carried out with time=0 as the initial release testing data. The stability storage freezer is a validated vapor phase LN₂ storage freezer which is set to maintain a temperature of ≤−135° C. For sterility timepoints, 10% of the batch size or 4 vials, whichever is greater, was tested. Test articles were thawed at 37° C. to mimic clinical thawing conditions.

As shown in Table 10, viability and activity of cryopreserved AB-101 was shown to be preserved through at least nine months.

TABLE 10
Long Term Viability and Activity of Cryopreserved AB-101

| | | | Cryopreserved (≤135° C.), Sample times | | | | | |
|---|---|---|---|---|---|---|---|---|
| | | | (months) | | | | | |
| | | Acceptance | 0 | 3 | 6 | 9 | 12 | 18 |
| Test Attribute | | Criterion | months | months | months | months | months | months |
| Cell Count | | 0.9-1.3 × | 1.3 × 10⁹ | 1.3 × | 1.4 × | 1.4 × | 1.3 × 10⁹ | 1.4 × 10⁹ |
| (cells/vial) | | 10⁹ | | 10⁹ | 10⁹ | 10⁹ | cells/vial | cells/vial |
| Cell Viability | | ≥70% | 96% | 93% | 94% | 93% | 90% | 87% |
| Endotoxin | | ≤5 | ≤1 | ≤1 | ≤1 | ≤1 | <1.0 | <1.0 |
| (EU/kg/hr) | | | | | | | | |
| Identity | CD3−, | ≥85% | 99.16% | 99.39% | 99.49% | 99.41% | 99.54% | 99.36% |
| | CD56+ % | | | | | | | |
| | CD56+, | ≥70% | 94.42% | 94.60% | 94.44% | 93.71% | 94.85% | 90.27% |
| | CD16+ % | | | | | | | |
| Purity | CD3+ % | ≤0.20% | 0.00% | 0.00% | 0.00% | 0.04% | 0.06% | 0.00% |
| | CD14+ % | ≤1.00% | 0.02% | 0.00% | 0.00% | 0.02% | 0.01% | 0.00% |
| | CD19+ % | ≤1.00% | 0.01% | 0.00% | 0.01% | 0.02% | 0.00% | 0.00% |

| | | | | | | | |
|---|---|---|---|---|---|---|---|
| Potency (killing at | ≥50% | 69.00% | 66.90% | 67.40% | 61.80% | 67.1 | 68.3 |
| 4 hours) | | | | | | | |

To understand the stability characteristics of AB-101 during handling just prior to administration, a “bedside” short-term stability study was performed. Samples were thawed, transferred to 10 mL syringes, filtered, and the contents stored in Falcon tubes, and kept at that temperature for defined time periods as shown. The collected product was then tested. Short-Term Stability Data for two lots of AB-101 is shown in Table 11.

TABLE 11
Short Term Stability Data for AB-101

| | | Lot | | | 15 | 30 | 60 | 90 | 120 | |
|---|---|---|---|---|---|---|---|---|---|---|
| Average data of 4 vials | | release | 0 min | 5 min | min | min | min | min | min | Flush |
| PG001 | Cell count | 1.18 | 1.10 | 1.11 | 1.11 | 1.10 | 1.12 | 1.07 | 1.03 | 0.07 |
| | (0.8-1.2 × | | | | | | | | | |
| | 10⁸ cells/mL) | | | | | | | | | |
| | Viability (%) | 93 | 94 | 94 | 94.75 | 94 | 93.5 | 93.5 | 93.5 | 93.25 |
| | CD3 − 56+ (%) | 99.53 | 99.53 | NT | NT | NT | 99.53 | NT | 97.58 | NT |
| | CD16 + CD56 | 93.24 | 97.74 | NT | NT | NT | 97.74 | NT | 97.43 | NT |
| | (%) | | | | | | | | | |
| PG002 | Cell count | 1.09 | 1.13 | 1.08 | 1.14 | 1.14 | 1.08 | 1.11 | 1.05 | 0.08 |
| | (0.8-1.2 × | | | | | | | | | |
| | 10⁸ cells/mL) | | | | | | | | | |
| | Viability (%) | 94 | 93.75 | 94.25 | 94.75 | 95.25 | 94.25 | 94.5 | 94 | 92.75 |
| | CD3 − 56+ (%) | 98.40 | 99.30 | NT | NT | NT | 99.27 | NT | 99.53 | NT |
| | CD16 + CD56 | 91.72 | 98.88 | NT | NT | NT | 99.55 | NT | 98.40 | NT |
| | (%) | | | | | | | | | |

### Example 11: Cord Blood NK Cells Selected for Kir-B and CD16 158 v/v Exhibit Low CD38 Expression after Expansion

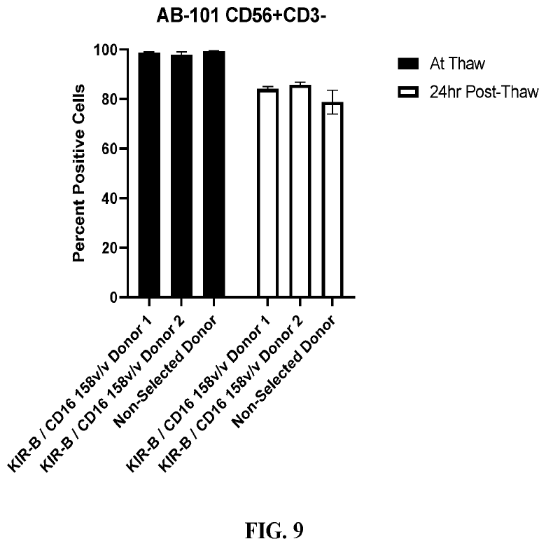

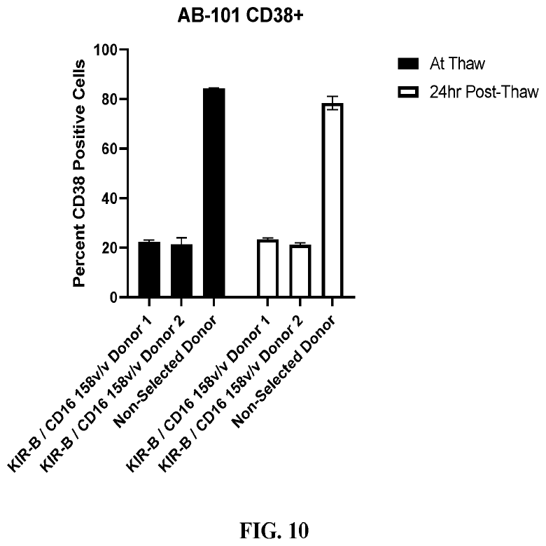

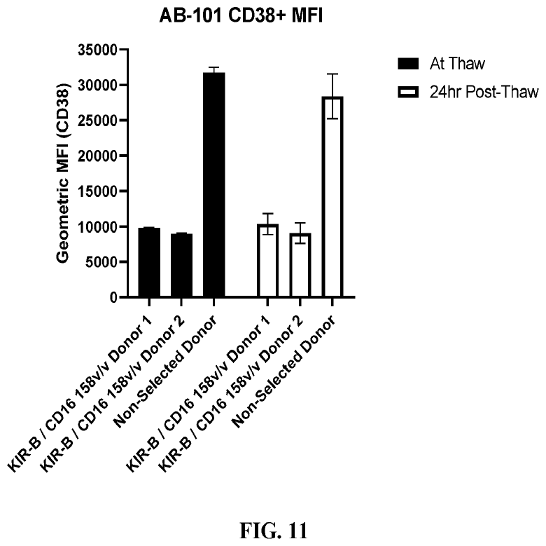

NK cells were expanded, as described in Example 6, using two different cord blood donors selected for KIR-B and CD16 158v/v to generate AB-101 cells, and from one non-selected donor (control). The purity of the resulting cells (percent CD56+CD3−) as measured by flow cytometry, is show in FIG. 9. As shown in FIG. 10 and FIG. 11, CD38 expression is lower in KIR-B/158 v/v NK cells as a population (percent positive, FIG. 10) and individually (mean fluorescence intensity of the positive cells, FIG. 11) compared to non-selected NK cells.

### Example 12: Surface Protein Expression of AB-101

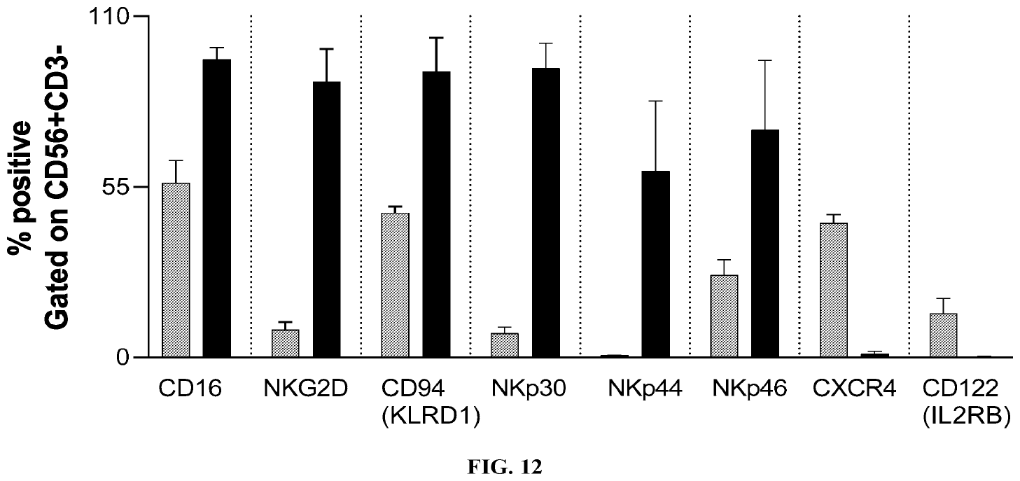

NK cells were expanded, as described in Example 6. Surface protein expression of the starting NK cell source (cord blood gated on CD56+/CD3-expression, n=3) was compared to the resulting expanded NK cells (n=16). As shown in FIG. 12, CD16 expression was high in the resulting cells, increased relative to the starting cells. Expression of NKG2D, CD94, NKp30, NKp44, and NKp46 was also increased, whereas expression of CXCR4 and CD122 was decreased.

### Example 13: Preloading AB-101 NK Cells with AFM13

As one example, 17.6 billion of AB-101 NK cells (cord-blood derived and expanded NK cells selected for V/V and KIR-B) (including 10% extra amount considering cell loss during centrifugation and formulation) were taken at harvest and transferred to the lab.

For preloading the AB-101 cells with AFM13, 4 billion cells were incubated in 40 mL culture media in the presence of 100 μg/mL of AFM13 for 30 minutes at ambient temperature, then each replicate was washed twice in 10 mL culture media, resuspended in 40 mL freeze media and cryopreserved in 1 mL aliquots. This preloading process results in AB-101 NK cells with AFM13 bound to (precomplexed with) CD16A molecules on the cells.

For the control AB-101 cells, 8 billion cells were resuspended in 80 mL Freezing medium, wherein the cells were frozen in 1 mL aliquots. The total number of cells required was 17.6 billion cells.

### Example 14: Antibody Dependent Cell-Mediated Cytotoxicity (ADCC) of AFM13 and AB-101 NK Cells

As one example, antibody-mediated target cell lysis by AB-101 NK cells in vitro was assessed by quantifying the release of calcein into cell culture supernatants from calcein-labeled Karpas-299 target cells after 4 hours. The assay was carried out with: 1) non-precomplexed (empty) AB-101 cells that had previously been washed and cryopreserved alone (“non-preloaded” condition): 2) “non-preloaded” AB-101 cells as in condition 1, but in combination with fresh (never frozen) AFM13 (“non-preloaded+fresh excess AFM13” condition); and 3) AB-101 cells preloaded with AFM13 (as described in Example 1), followed by removal of unbound AFM13 (i.e., washing) and subsequent cryopreservation. Prior to the assay, cryopreserved AB-101 cells were swiftly thawed at 37° C. and washed in PBS buffer supplemented with 2% FCS and 0.6% citrate-dextrose solution. The assay was carried out with AB-101 cells derived from two different cord units (MCB1 and MCB2), n=3 and n=4, respectively.

Target cells were labeled with 10 μM calcein AM for 30 min in RPMI 1640 medium without FCS at 37° C. After gentle washing, calcein-labeled cells were resuspended in complete RPMI medium (i.e., RPMI 1640 medium supplemented with 10% h.i. FCS, 2 mM L glutamine. 100 U/mL penicillin G sodium. 100 μg/mL streptomycin sulfate) at a density of 1×10⁵/mL. 1×10⁴ target cells were then seeded in individual wells of a round-bottom 96-well microtiter plate and, if not mentioned otherwise, mixed with thawed empty or preloaded AB-101 NK cells at decreasing effector-to-target cell (E:T) ratios starting at 10:1 followed by two-fold serial dilutions. If indicated. 10 μg/mL of AFM13 were added to individual wells of empty or preloaded AB-101 NK cells to a total volume of 200 μL/well in duplicates.

Spontaneous calcein-release and maximal release were determined in quadruplicate on each plate. Spontaneous release was determined by incubation of target cells in the absence of effector NK cells and in the absence of AFM13. Maximal release was achieved by adding Triton X 100 to a final concentration of 1% in the absence of effector cells and in the absence of antibodies. After centrifugation for 2 min at 200×g, microtiter plates were incubated for 4 h at 37° C. in a humidified atmosphere with 5% CO2. Following incubation. 100 μL cell-free cell culture supernatant was harvested from each well after centrifugation for 5 min at 500×g and transferred to black flat-bottom 96-well microtiter plates. Fluorescence counts of released calcein were measured at 520 nm using a multimode plate reader. Specific cell lysis was calculated according to the following formula: [fluorescence (sample)−fluorescence (spontaneous)]/[fluorescence (maximum)−fluorescence (spontaneous)]×100% wherein “Fluorescence (spontaneous)” and “Fluorescence (maximum)” are defined as fluorescence in absence of effector cells and antibodies and fluorescence induced by the addition of Triton X 100, respectively.

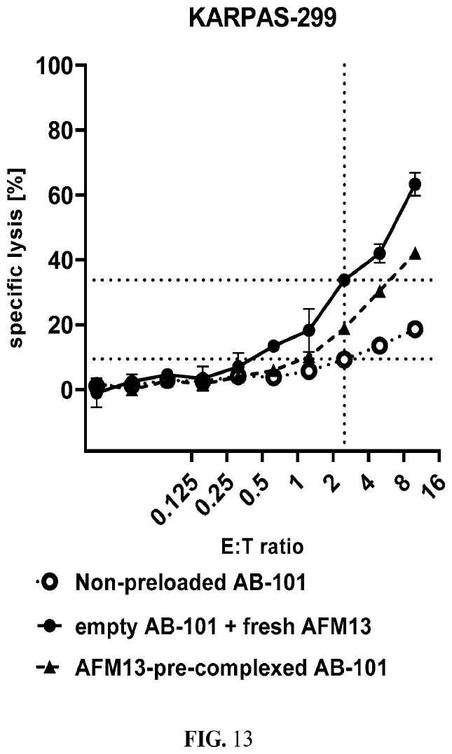

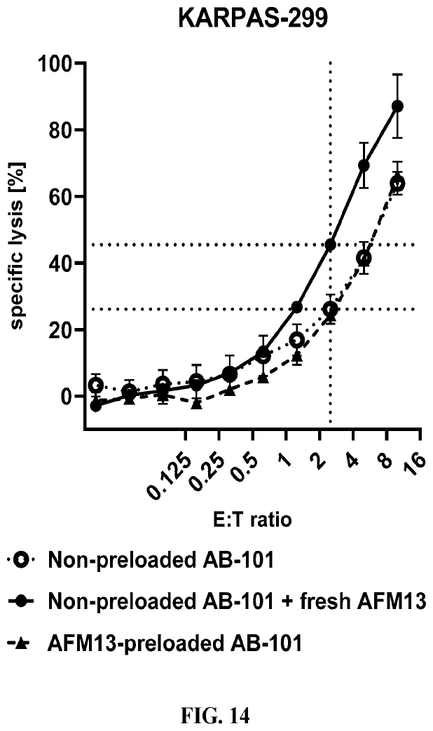

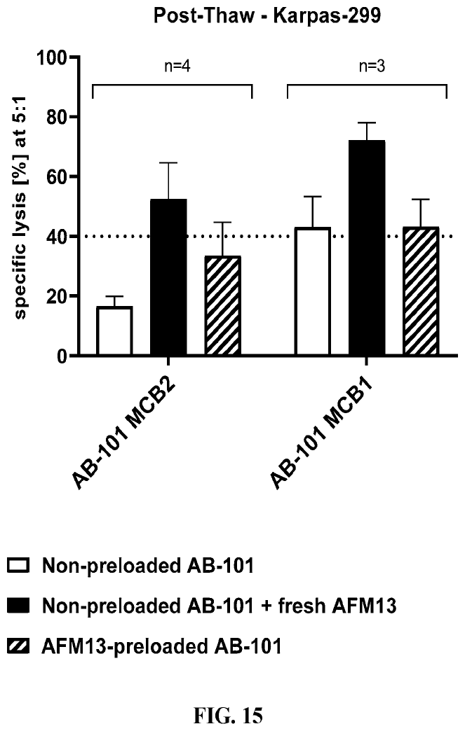

As shown in FIG. 13. FIG. 14, and FIG. 15. ADCC activity of AB-101 cells in combination with AFM13 exhibited an ADCC response as strong or stronger than AB-101 alone, both when AFM13 was added fresh and when it was pre-loaded prior to cryopreservation.

### Example 15: Retention of Bound AFM13 after Cryopreservation by AFM13-Preloaded Cryopreserved AB-101 NK Cells

Retention of bound AFM13 after cryopreservation by AFM13-preloaded cryopreserved AB-101 NK cells (here referred to as occupancy) was assessed by flow cytometry. Preloading procedures and thawing after cryopreservation was performed as described in Example 14.

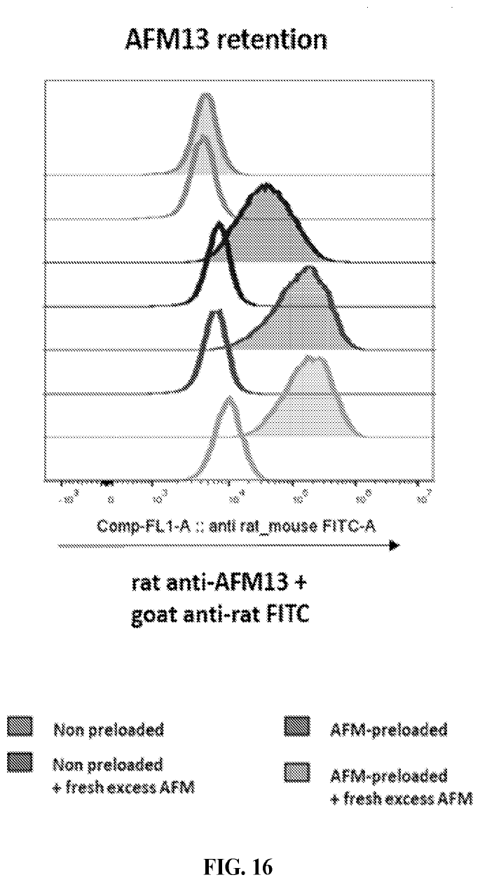

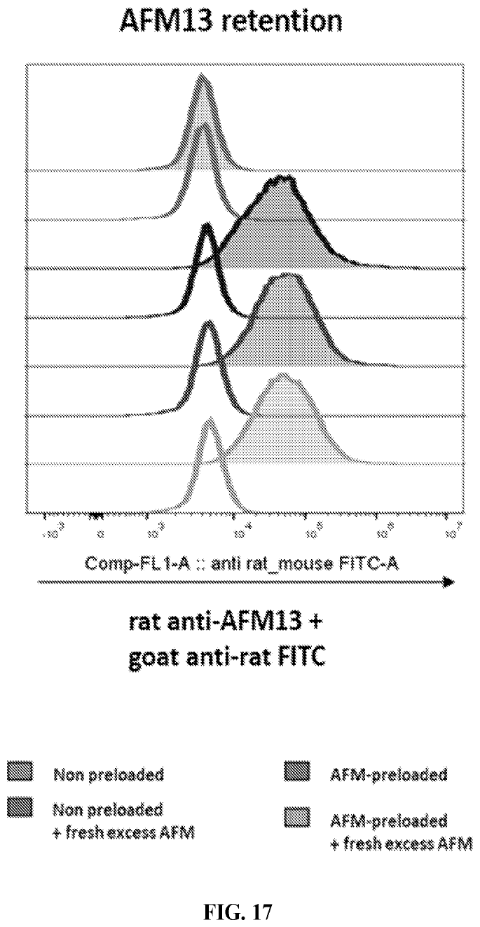

Thawed empty or AFM13-preloaded AB-101 NK cells were washed and resuspended in FACS buffer (i.e., PBS supplemented with 2% FCS and 0.1% sodium azide) and seeded in individual wells of a round-bottom 96-well microtiter plate at 2-4×10⁵ cells. Cells were incubated with rat anti-AFM13 clone 7 antibody (5 μg/mL. Affimed GmbH) diluted in FACS buffer or FACS alone for 30 min in the dark at 4° C. followed by washing and subsequent incubation with goat anti-rat FITC (1/100. Dianova) for 30 min in the dark at 4° C. After final washing twice, cells were measured on a CytoFlex S flow cytometer (Beckman Coulter) followed by analysis by FlowJo software. Positive detection of AFM13 on AFM13-preloaded AB-101 cells was determined in comparison of staining with goat anti-rat FITC only as well as relative to empty AB-101 cells (FIG. 16 and FIG. 17). Filled histograms represent anti-AFM13+secondary antibody; open histograms represent secondary antibody only. The fluorescence intensity of bound AFM13 on pre-complexed AB-101 cells was as high as that of empty AB-101 cells freshly incubated with AFM13, indicating maximal loading/saturation.

For evaluation of maximal loading/saturation, control empty and control preloaded AB-101 were first freshly incubated with AFM13 (10 μg/mL) prior to the staining procedures described above.

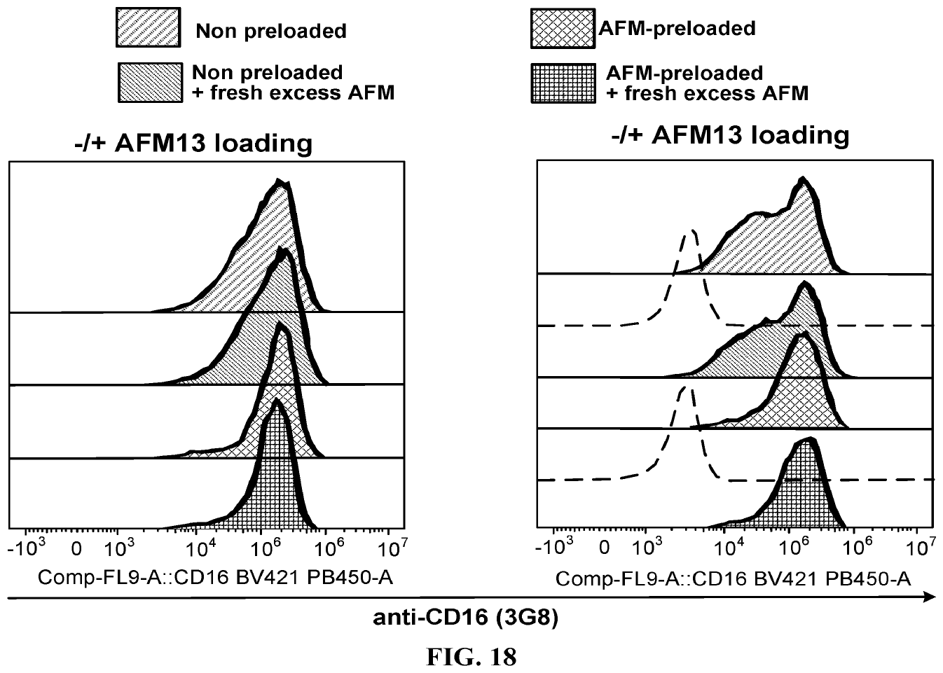

Additionally, empty. AFM13-preloaded AB-101 NK cells were incubated with mouse anti-human CD16 BV421 (clone 3G8, 1/100, Biolegend), with mouse anti-human CD56 BV785 (clone 5.1H11, 1/100. Biolegend) and analyzed compared to corresponding concentration-matched mouse isotype controls (all from Biolegend). As shown in FIG. 18 (left: MCB2; right: MCB1). CD16 expression is detected on preloaded cryopreserved AB-101 cells after thawing. Filled histograms represent CD16 staining; open histograms represent isotype control antibody staining only. The detection of uniform high AFM13 binding relative to uniform high CD16 expression suggests saturated binding of CD16 by AFM13.

### Example 16: AFM13 Mediated NK Fratricide Detection

NK-NK cell lysis (i.e., NK fratricide), potentially mediated by cross-linking of two CD16 molecules or of CD30 and CD16 (by AFM13) on two adjacent NK cells, was assessed in vitro by quantifying the release of calcein into cell culture supernatants from calcein-labeled NK cell after 4 hour co-culture with autologous non-labelled NK cells. Preloading procedures and thawing after cryopreservation was performed as described in Example 14.

Target empty or preloaded AB-101 NK cells were labelled with calcein as described in Example 14. 5×10⁴ target NK cells were then seeded in individual wells of a round-bottom 96-well microtiter plate and, if not mentioned otherwise, mixed with effector empty or preloaded AB-101 NK cells at 1:1 E:T ratio. If indicated. 10 μg/mL of AFM13 were added to individual wells to a total volume of 200 μL/well in duplicates.

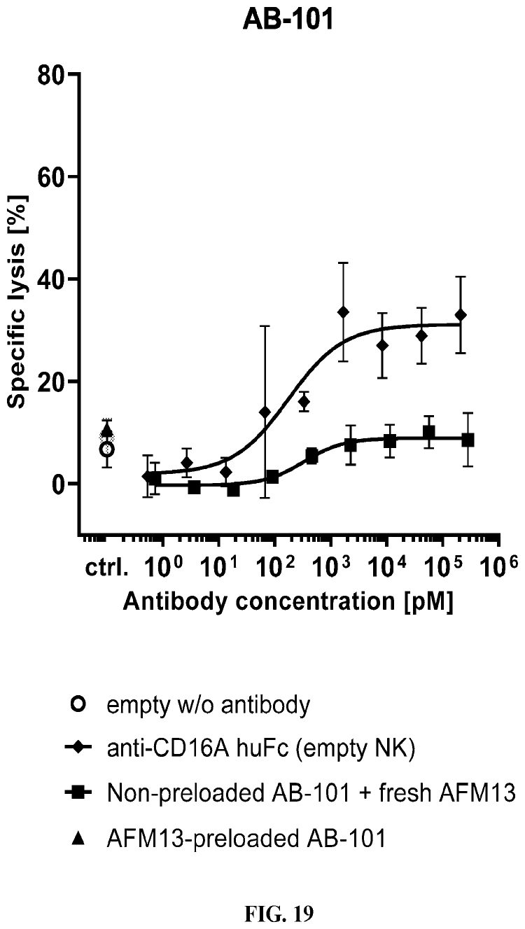

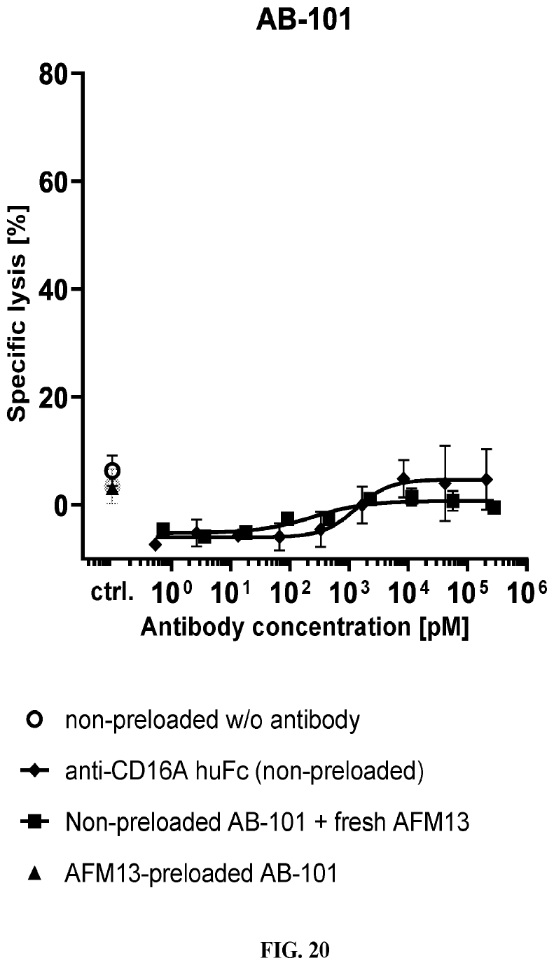

The following conditions were tested: a) Target calcein-labelled AFM13-preloaded AB-101 cells with effector AFM13-preloaded AB-101 cells, b) Target calcein-labelled empty AB-101 cells with effector empty AB-101 cells, and c) Target calcein-labelled empty AB-101 cells with effector empty AB-101 cells in the presence of excess (no wash) AFM13. As shown in FIG. 19 (MCB2) and FIG. 20 (MCB1). AB-101 cells used in combination with AFM13 exhibited low levels of NK fratricide both if preloaded and if freshly added in excess (′co-admin), despite the significant expression of CD30 (and CD16) on AB-101 cells. In contrast, in primary buffy coat-derived NK cells. AFM13 can result in dose-dependent NK fratricide.

### Example 17: NK Cell Activation in Response to Target Cells by AFM13-Preloaded Cryopreserved AB-101 NK Cells

To monitor NK cell activation, up-regulation of CD107a (marker for NK cell degranulation) and intracellular IFN-γ expression were assessed in response to Karpas-299 target cell lines and AFM13. Preloading procedures and thawing after cryopreservation were performed as described in Example 14.

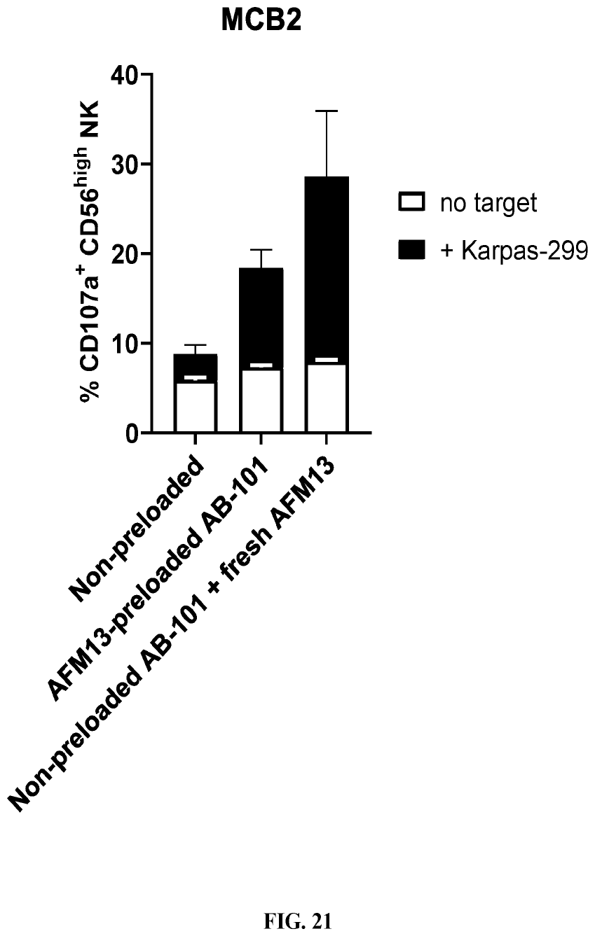

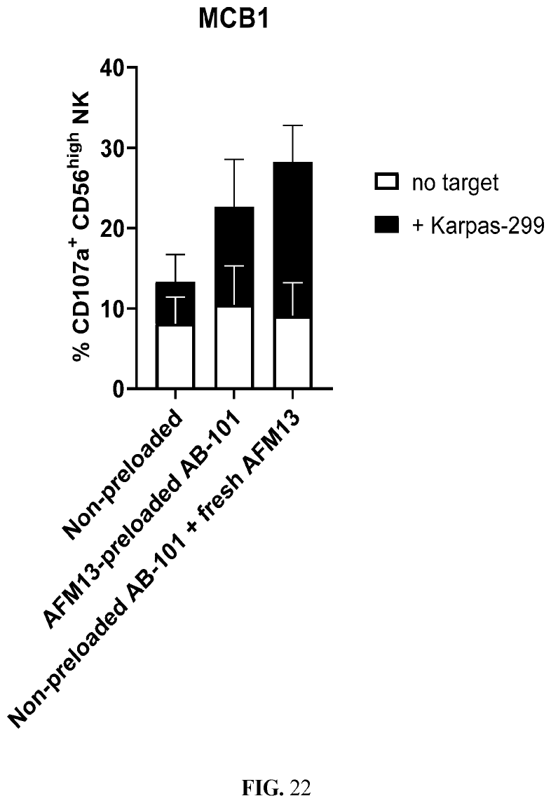

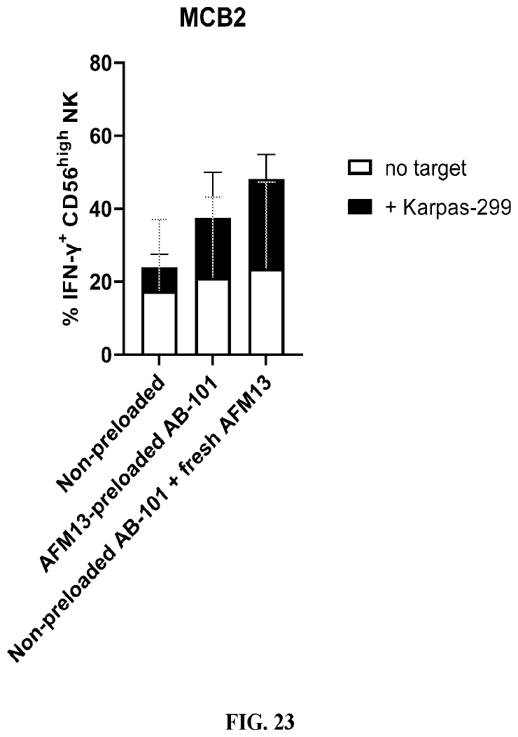

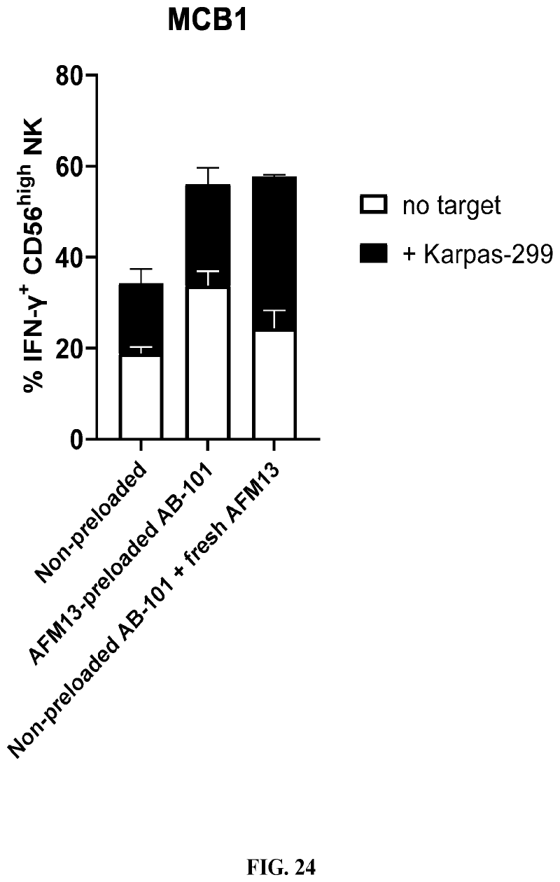

Empty (negative control) and preloaded AB-101 NK cells, and AB-101 NK cells with 0.5 g/mL added (fresh) AFM13 were co-cultured with and without tumor target cells at 1:1 cell ratio (each 5×10⁴ cells) in the presence of anti-CD107a-FITC (1/100 v/v, clone H4A3. Biolegend) and GolgiPlug (1/1000 v/v. BD Bioscience) in complete RPMI 1640 medium in round-bottom 96-well plates for 4 hours. The percentage of NK cell positive for extracellular CD107a+ (FIG. 21 (MCB2) and FIG. 22 (MCB1)) or intracellular IFN γ+NK cells (FIG. 23 (MCB2) and FIG. 24 (MCB1)) was determined by flow cytometry. Flow cytometry staining was performed as described in Example 14. As shown by FIG. 21. FIG. 22. FIG. 23, and FIG. 24. AFM13-preloaded cells and empty AB-101 cells supplemented with AFM13 exhibited degranulation and increased production of IFN-γ, specifically in response to Karpas-299 target cells, while AFM13-preloaded AB-101 cells and empty AB-101 cells supplemented with AFM13 did not exhibit significant degranulation or IFN-γ expression in the absence of target cells.

### Example 18: Viability of Cryopreserved AFM13 Preloaded AB-101

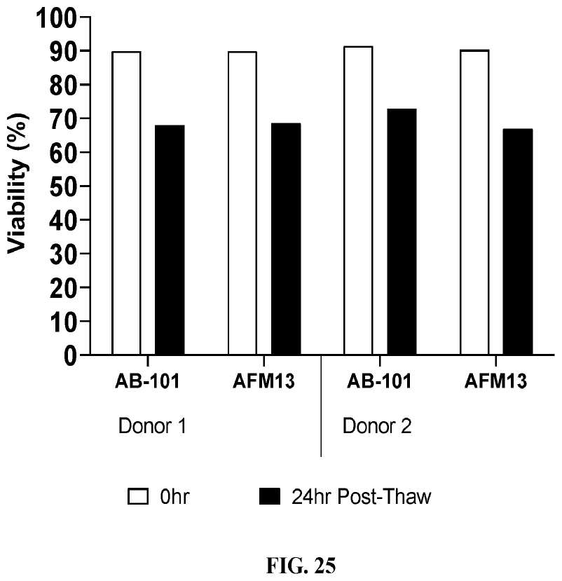

Viability analysis of cryopreserved AFMs-preloaded AB-101 (vs cryopreserved non-loaded) was performed after thawing and after additional 24-hour culture. As shown in FIG. 25, viability of AB-101 cells derived from two separate donors was over 80% post-thawing, and over 60% 24 hours post-thawing.

### Example 19: Assessment of AFM13 in Combination with AB-101 In Vivo: Karpas-299/Luc Cells i.v. In hIL-15 NOG Mice

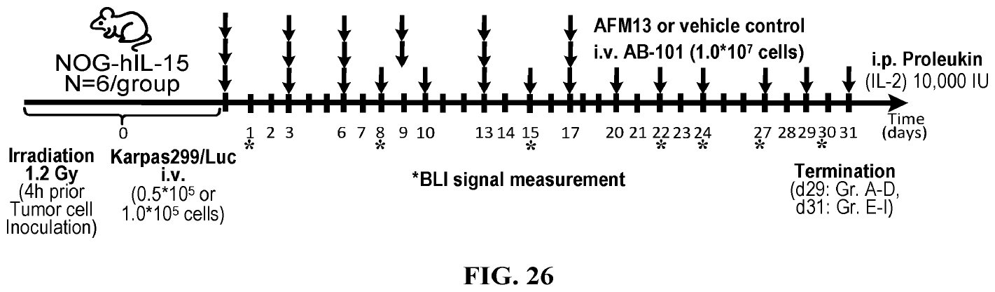

The human non-Hodgkin CD30-positive large cell lymphoma cell line Karpas-299 was established from the peripheral blood of a patient with T cell non-Hodgkin lymphoma, classed as CD30-positive (ALCL), with the NPM-ALK fusion gene. The cell line was transfected with the luciferase transcript for bioluminescence imaging. The lymphoma cell line Karpas-299/Luc model was used to assess the efficacy of AB-101 in combination with AFM13. In brief, on study day 0, hIL-15 NOG mice were irradiated with 1.2 Gy followed by intraperitoneal (i.p.) proleukin (IL-2) 10,000 IU administration. Four hours following irradiation, the animals were inoculated with 0.5×10⁵ or 1×10⁵ Karpas-299/Luc cells. Immediately following tumor inoculation, animals were administered intravenously (i.v) with AB-101 alone (1×10⁷ cells) or with AB-101 followed by AFM13 (10 mg/kg) by alternating tail veins for each administration. Supplementation with IL-2 i.p. continued every second day and treatment with AB-101 and AFM13 continued every third day for a total of 6 doses and was administered after IL-2 supplementation when scheduled for the same day (FIG. 26, Table 12). Mice were imaged weekly for bioluminescence (BLI) starting from day 1. Body weights and general health conditions were recorded throughout the entire study.

TABLE 12
Experimental Design

| | No. | | Application | Sequence | | Effector: |
|---|---|---|---|---|---|---|
| Group | mice | Treatment | Route | Days | Dose | Target ratio |
| A | 6 | Karpas-299/Luc cells | i.v. | d0 | 1 × 10⁵ | |

| | | | | | |
|---|---|---|---|---|---|
| Vehicle (freeze | i.v. | from d0: | 0.1 | ml | N/A |
| media) | | Q3D x6 | | | |

| | | | | | |
|---|---|---|---|---|---|
| B | 6 | Karpas-299/Luc cells | i.v. | d0 | 1 × 10⁵ |

| | | | | | |
|---|---|---|---|---|---|
| Vehicle (freeze | i.v. | from d0: | 0.1 | ml | N/A |
| media) | | Q3D x6 | | | |
| Proleukin (IL-2) | i.p. | Q2D Mo- | 10,000 | IU | |
| | | Fr | | | |

| | | | | | | |
|---|---|---|---|---|---|---|
| C | 6 | Karpas-299/Luc cells | i.v. | d0 | 1 × 10⁵ | |
| | | AB-101 | i.v. | from d0: | 1 × 10⁷ | 100:1 |

Q3D x6

| | | | | | | |
|---|---|---|---|---|---|---|
| D | 6 | Karpas-299/Luc cells | i.v. | d0 | 1 × 10⁵ | |
| | | AB-101 | i.v. | from d0: | 1 × 10⁷ | 100:1 |

| | | | | |
|---|---|---|---|---|
| | | Q3D x6 | | |
| Proleukin (IL-2) | i.p. | Q2D Mo- | 10,000 | IU |
| | | Fr | | |

| | | | | | | |
|---|---|---|---|---|---|---|
| E | 6 | Karpas-299/Luc cells | i.v. | d0 | 1 × 10⁵ | |
| | | AB-101 | i.v. left | from d0: | 1 × 10⁷ | 100:1 |

| | | | | |
|---|---|---|---|---|
| | | Q3D x6 | | |
| AFM13 | i.v. right | from d0: | 10 | mg/kg |
| | | Q3D x6 | | |

| | | | | | | |
|---|---|---|---|---|---|---|
| F | 6 | Karpas-299/Luc cells | i.v. | d0 | 1 × 10⁵ | |
| | | AB-101 | i.v. left | from d0: | 1 × 10⁷ | 100:1 |

| | | | | |
|---|---|---|---|---|
| | | Q3D x6 | | |
| AFM13 | i.v. right | from d0: | 10 | mg/kg |
| | | Q3D x6 | | |
| Proleukin (IL-2) | i.p. | Q2D Mo- | 10,000 | IU |
| | | Fr | | |

*Groups G-I were the same as D-F with an E:T ratio of 200:1, 0.5 × 10⁵ Karpas-299/Luc and 1 × 10⁷ AB-101

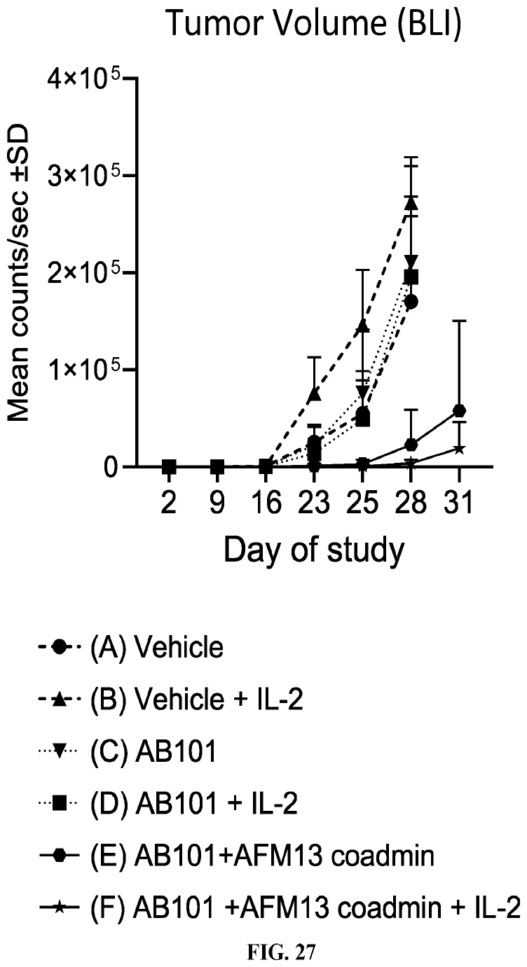

The intravenous injection of 0.5×10⁵ or 1×10⁵ Karpas-299/Luc cells resulted in a disseminated lymphoma engraftment in the mice. The engraftment was detected by BLI on day 1 after the i.v. inoculation of the cells. On day 27 some mice showed progressive lymphoma growth and had to be sacrificed. Further progressive lymphoma growth was observed from day 27 to day 29 in the groups that had received the higher dose of Karpas-299/Luc (Group A-D). The remaining groups were further followed until day 31. Enhanced anti-tumor efficacy was observed in AB-101/AFM13 treated groups compared to control groups at several timepoints throughout the study (FIG. 27) In the lower dose Karpas-299/Luc groups (group G-I), there was variation in BLI which did not reach significance on any specific day in group-wise comparison.

Necropsy confirmed progressive lymphoma growth in all mice in vehicle control groups (A and B) and in mice receiving AB-101 alone (C and D), with dissemination in peritoneum as well as around superficial axillary and inguinal lymph nodes. Individual animals in AB-101+AFM13 groups (E, H, and I) were free of macroscopically visible tumor lesions.

In groups which received AB-101 (groups C-I), after 4 doses with the schedule Q3D (do, d3, d6 and d9) animals showed on day 10 mean body weight losses of ˜10-13%. Following a change in dose schedule to Q4D (d13 and d17), mice in group C to F recovered over the following days. Mice in groups G to I showed ˜6-12% body weight change on day 15 following NK cell inoculation on day 13. Therefore, the last scheduled administration on d17 was not performed in those groups.

Anti-tumor efficacy was observed in groups receiving the combination of AFM13 and AB-101, with statistical significance compared to control groups. Differences in tumor outgrowth were confirmed by macroscopic findings of reduced tumor masses in inguinal and axillary lymph nodes and the abdominal cavity of animals that received AB-101 and AFM13. In conclusion, the combination of AFM13 and AB-101 demonstrated significant anti-tumor efficacy in the Karpas-299/Luc xenograft tumor model.

SEQUENCES

| SEQ ID NO: and | |
|---|---|
| DESCRIPTION | SEQUENCE |
| SEQ ID NO: 1 | MEYASDASLDPEAPWPPAPRARACRVLPWALVAGLLLLLLLAAACAVELA |
| Sequence of 4- | CPWAVSGARASPGSAASPRLREGPELSPDDPAGLLDLRQGMFAQLVAQNV |
| 1BBL that can | LLIDGPLSWYSDPGLAGVSLTGGLSYKEDTKELVVAKAGVYYVFFQLELR |
| be expressed by | RVVAGEGSGSVSLALHLQPLRSAAGAAALALTVDLPPASSEARNSAFGFQ |
| feeder cells | GRLLHLSAGQRLGVHLHTEARARHAWQLTQGATVLGLFRVTPEIPAGLPS |
| | PRSE |
| SEQ ID NO: 2 | MALPVTALLLPLALLLHAARPQDRHMIRMRQLIDIVDQLKNYVNDLVPEF |
| Sequence of a | LPAPEDVETNCEWSAFSCFQKAQLKSANTGNNERIINVSIKKLKRKPPST |
| membrane | NAGRRQKHRLTCPSCDSYEKKPPKEFLERFKSLLQKMIHQHLSSRTHGSE |
| bound IL- | DSAKPTTTPAPRPPTPAPTIASQPLSLRPEACRPAAGGAVHTRGLDFACD |
| 21(mbIL-21) | IYIWAPLAGTCGVLLLSLVITLY |
| that can be | |
| expressed by | |
| feeder cells | |
| SEQ ID NO: 3 | MSTESMIRDVELAEEALPKKTGGPQGSRRCLFLSLFSFLIVAGATTLFCL |
| Sequence of a | LHFGVIGPQREEFPRDLSLISPLAQPVRSSSRTPSDKPVAHVVANPQAEG |
| mutated TNF | QLQWLNRRANALLANGVELRDNQLVVPSEGLYLIYSQVLFKGQGCPSTHV |
| alpha (mTNF-a) | LLTHTISRIAVSYQTKVNLLSAIKSPCQRETPEGAEAKPWYEPIYLGGVF |
| that can be | QLEKGDRLSAEINRPDYLDFAESGQVYFGIIAL |
| expressed by | |
| feeder cells | |
| SEQ ID NO: 4 | MERVQPLEENVGNAARPRFERNKLLLVASVIQGLGLLLCFTYICLHFSAL |
| Sequence of | QVSHRYPRIQSIKVQFTEYKKEKGFILTSQKEDEIMKVQNNSVIINCDGE |
| OX40L that can | YLISLKGYFSQEVNISLHYQKDEEPLFQLKKVRSVNSLMVASLTYKDKVY |
| be expressed by | LNVTTDNTSLDDFHVNGGELILIHQNPGEFCVL |
| feeder cells | |
| SEQ ID NO: 5 | MKWVTFISLLFLESSAYSRGVERRDAHKSEVAHRFKDLGEENFKALVLIA |
| Human Albumin | FAQYLQQCPFEDHVKLVNEVTEFAKTCVADESAENCDKSLHTLFGDKLCT |
| | VATLRETYGEMADCCAKQEPERNECFLQHKDDNPNLPRLVRPEVDVMCTA |
| | FHDNEETFLKKYLYEIARRHPYFYAPELLFFAKRYKAAFTECCQAADKAA |
| | CLLPKLDELRDEGKASSAKQRLKCASLQKFGERAFKAWAVARLSQRFPKA |
| | EFAEVSKLVTDLTKVHTECCHGDLLECADDRADLAKYICENQDSISSKLK |
| | ECCEKPLLEKSHCIAEVENDEMPADLPSLAADFVESKDVCKNYAEAKDVF |
| | LGMFLYEYARRHPDYSVVLLLRLAKTYETTLEKCCAAADPHECYAKVEDE |
| | FKPLVEEPQNLIKQNCELFEQLGEYKFQNALLVRYTKKVPQVSTPTLVEV |
| | SRNLGKVGSKCCKHPEAKRMPCAEDYLSVVLNQLCVLHEKTPVSDRVTKC |
| | CTESLVNRRPCFSALEVDETYVPKEFNAETFTFHADICTLSEKERQIKKQ |
| | TALVELVKHKPKATKEQLKAVMDDFAAFVEKCCKADDKETCFAEEGKKLV |
| | AASQAALGL |
| SEQ ID NO: 6 | SYYMH |
| CD16 CDR H1 | |
| SEQ ID NO: 7 | IINPSGGSTSYAQKFQG |
| CD16 CDR H2 | |
| SEQ ID NO: 8 | GSAYYYDFADY |
| CD16 CDR H3 | |
| SEQ ID NO: 9 | GGHNIGSKNVH |
| CD16 CDR L1 | |
| SEQ ID NO: 10 | QDNKRPS |
| CD16 CDR L2 | |
| SEQ ID NO: 11 | QVWDNYSVL |
| CD16 CDR L3 | |
| SEQ ID NO: 12 | TYTIH |
| CD30 CDR H1 | |
| SEQ ID NO: 13 | YINPSSGYSDYNQNFKG |
| CD30 CDR H2 | |
| SEQ ID NO: 14 | RADYGNYEYTWFAY |
| CD30 CDR H3 | |
| SEQ ID NO: 15 | KASQNVGTNVA |
| CD30 CDR L1 | |
| SEQ ID NO: 16 | SASYRYS |
| CD30 CDR L2 | |
| SEQ ID NO: 17 | QQYHTYPLT |
| CD30 CDR L3 | |
| SEQ ID NO: 18 | QVQLVQSGAEVKKPGESLKVSCKASGYTFTSYYMHWVRQAPGQGLEWMGI |
| CD16x CD30 | INPSGGSTSYAQKFQGRVTMTRDTSTSTVYMELSSLRSEDTAVYYCARGS |
| bispecific Ab | AYYYDFADYWGQGTLVTVSSGGSGGSGGSDIVMTQSPKFMSTSVGDRVTV |
| construct | TCKASQNVGTNVAWFQQKPGQSPKVLIYSASYRYSGVPDRFTGSGSGTDF |
| | TLTISNVQSEDLAEYFCQQYHTYPLTFGGGTKLEINGGSGGSGGSQVQLQ |
| | QSGAELARPGASVKMSCKASGYTFTTYTIHWVRQRPGHDLEWIGYINPSS |
| | GYSDYNQNFKGKTTLTADKSSNTAYMQLNSLTSEDSAVYYCARRADYGNY |
| | EYTWFAYWGQGTTVTVSSGGSGGSGGSSYVLTQPSSVSVAPGQTATISCG |
| | GHNIGSKNVHWYQQRPGQSPVLVIYQDNKRPSGIPERFSGSNSGNTATLT |
| | ISGTQAMDEADYYCQVWDNYSVLFGGGTKLTVL |
| SEQ ID NO: 19 | QVQLVQSGAEVKKPGESLKVSCKASGYTFTSYYMHWVRQAPGQGLEWMGI |
| VH CD16 | INPSGGSTSYAQKFQGRVTMTRDTSTSTVYMELSSLRSEDTAVYYCARGS |
| | AYYYDFADYWGQGTLVTVSS |
| SEQ ID NO: 20 | SYVLTQPSSVSVAPGQTATISCGGHNIGSKNVHWYQQRPGQSPVLVIYQD |
| VL CD16 | NKRPSGIPERFSGSNSGNTATLTISGTQAMDEADYYCQVWDNYSVLFGGG |
| | TKLTVL |
| SEQ ID NO: 21 | QVQLQQSGAELARPGASVKMSCKASGYTFTTYTIHWVRQRPGHDLEWIGY |
| VH CD30 | INPSSGYSDYNQNFKGKTTLTADKSSNTAYMQLNSLTSEDSAVYYCARRA |
| | DYGNYEYTWFAYWGQGTTVTVSS |
| SEQ ID NO: 22 | DIVMTQSPKFMSTSVGDRVTVTCKASQNVGTNVAWFQQKPGQSPKVLIYS |
| VL CD30 | ASYRYSGVPDRFTGSGSGTDFTLTISNVQSEDLAEYFCQQYHTYPLTFGG |
| | GTKLEIN |
| SEQ ID NO: 23 | SFFPPGYQ |
| C-terminal | |
| CD16a | |
| SEQ ID NO: 24 | GMRTEDLPKAVVFLEPQWYRVLEKDSVTLKCQGAYSPEDNSTQWFHNESL |
| Human CD16A | ISSQASSYFIDAATVDDSGEYRCQTNLSTLSDPVQLEVHIGWLLLQAPRW |
| | VFKEEDPIHLRCHSWKNTALHKVTYLQNGKGRKYFHHNSDFYIPKATLKD |
| | SGSYFCRGLFGSKNVSSETVNITITQGLAVSTISSFFPPGYQ |
| SEQ ID NO: 25 | HHHHHH |
| hexa-histidine | |
| tag | |
| SEQ ID NO: 26 | GGSGGSGGS |
| L1 | |
| SEQ ID NO: 27 | GGSGGSGGS |
| L2 | |
| SEQ ID NO: 28 | GGSGGSGGS |
| L3 | |

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating a patient suffering from a CD30+ cancer, the method comprising:
administering to the patient a first pharmaceutical composition comprising a natural killer cell (NK cell) comprising a KIR-B haplotype and expression of a CD16 molecule; and
administering to the patient a second pharmaceutical composition comprising a bispecific antibody or antigen binding fragment thereof comprising a first binding domain that specifically binds to CD16 (FcγRIII) and a second binding domain that specifically binds to CD30,
wherein the first binding domain that specifically binds to CD16 comprises:
a light chain variable domain (VL_CD16A) comprising a light chain complementarity determining region 1 (CDRL1) comprising SEQ ID NO: 9, a light chain complementarity determining region 2 (CDRL2) comprising SEQ ID NO: 10: a light chain complementarity determining region 3 (CDRL3) comprising SEQ ID NO: 11; and
a heavy chain variable domain (VH_CD16A) comprising a heavy chain complementarity determining region 1 (CDRH1 comprising SEQ ID NO: 6: a heavy chain complementarity determining region 2 (CDRH2) comprising SEQ ID NO: 7; and a heavy chain complementarity determining region 3 (CDRH3) comprising SEQ ID NO: 8; and
wherein the second binding domain that specifically binds to CD30 comprises:
a light chain variable domain (VL_CD30) comprising a light chain complementarity determining region 1 (CDRL1) comprising SEQ ID NO: 15, a light chain complementarity determining region 2 (CDRL2) comprising SEQ ID NO: 16: a light chain complementarity determining region 3 (CDRL3) comprising SEQ ID NO: 17; and
a heavy chain variable domain (VH_CD30) comprising a heavy chain complementarity determining region 1 (CDRH1 comprising SEQ ID NO: 12: a heavy chain complementarity determining region 2 (CDRH2) comprising SEQ ID NO: 13; and a heavy chain complementarity determining region 3 (CDRH3) comprising SEQ ID NO: 14.

2. The method of claim 1, wherein the NK cell is a cord blood-derived NK cell.

3. The method of claim 2, wherein the cord blood-derived NK cell has been produced by a method comprising:
(a) providing a sample of cord blood cells comprising natural killer cells;
(b) depleting the cells of CD3 (+) cells or enriching the seed cells for NK cells by positive selection;
(c) expanding the natural killer cells by culturing the seed cells with a first plurality of cells from an inactivated CD4 (+) T cell line in a medium comprising IL-2,
to produce the cord blood-derived natural killer cell.

4. The method of claim 3, wherein the inactivated CD4 (+) T cell line expresses at least one gene selected from the group consisting of a 4-1BBL gene, a membrane-bound IL-21 (mbIL-21) gene, an OX40L gene, and a mutated TNF-α gene.

5. The method of claim 4, wherein the inactivated CD4 (+) T cell line expresses a 4-1BBL gene, a membrane-bound IL-21 (mbIL-21) gene, and a mutated TNF-α gene.

6. The method of any one of claims 1-5, wherein the medium comprising IL-2 further comprises a T-cell stimulating antibody selected from the group consisting of OKT3, UCHT1, HTa, or a combination thereof.

7. The method of any one of claims 1-6, wherein the CD16 molecule is a CD16A molecule.

8. The method of any one of claims 1-7, wherein the CD16 molecule comprises a V/V polymorphism at F158.

9. The method of any one of claims 1-8, wherein the first binding domain that specifically binds to CD16 specifically binds to CD16A.

10. The method of any one of claims 1-8, wherein the natural killer cell is a population of natural killer cells.

11. The method of claim 10, wherein the population of natural killer cells comprises at least 60%, e.g., at least 70%, at least 80%, at least 90% at least 95%, at least 99%, or 100% CD16+ cells.

12. The method of any one of claims 10-11, wherein the population of natural killer cells comprises at least 60%, e.g., at least 70%, at least 80%, at least 90% at least 95%, at least 99%, or 100% NKG2D+ cells.

13. The method of any one of claims 10-12, wherein the population of natural killer cells comprises at least 60%, e.g., at least 70%, at least 80%, at least 90% at least 95%, at least 99%, or 100% NKp46+ cells.

14. The method of any one of claims 10-13, wherein the population of natural killer cells comprises at least 60%, e.g., at least 70%, at least 80%, at least 90% at least 95%, at least 99%, or 100% NKp30+ cells.

15. The method of any one of claims 10-14, wherein the population of natural killer cells comprises at least 60%, e.g., at least 70%, at least 80%, at least 90% at least 95%, at least 99%, or 100% DNAM-1+ cells.

16. The method of any one of claims 10-15, wherein the population of natural killer cells comprises at least 60%, e.g., at least 70%, at least 80%, at least 90% at least 95%, at least 99%, or 100% NKp44+ cells.

17. The method of any one of claims 10-16, wherein the population of natural killer cells comprises less than 20%, e.g., 10% or less, 5% or less, 1% or less, 0.5% or less, or 0% CD3+ cells.

18. The method of any one of claims 10-17, wherein the population of natural killer cells comprises less than 20% or less, e.g., 10% or less, 5% or less, 1% or less, 0.5% or less, or 0% CD14+ cells.

19. The method of any one of claims 10-18, wherein the population of natural killer cells comprises less than 20% or less, e.g., 10% or less, 5% or less, 1% or less, 0.5% or less, or 0% CD19+ cells.

20. The method of any one of claims 10-19, wherein the population of natural killer cells comprises less than 20% or less, e.g., 10% or less, 5% or less, 1% or less, 0.5% or less, or 0% CD38+ cells.

21. The method of any one of claims 10-20, wherein the population of NK cells comprises at least 100 million expanded natural killer cells, e.g., 200 million, 250 million, 300 million, 400 million, 500 million, 600 million, 700 million, 750 million, 800 million, 900 million, 1 billion, 2 billion, 3 billion, 4 billion, 5 billion, 6 billion, 7 billion, 8 billion, 9 billion, 10 billion, 15 billion, 20 billion, 25 billion, 50 billion, 75 billion, 80 billion, 9-billion, 100 billion, 200 billion, 250 billion, 300 billion, 400 billion, 500 billion, 600 billion, 700 billion, 800 billion, 900 billion, 1 trillion, 2 trillion, 3 trillion, 4 trillion, 5 trillion, 6 trillion, 7 trillion, 8 trillion, 9 trillion, or 10 trillion expanded natural killer cells.

22. The method of any one of claims 10-21, wherein the population of NK cells is produced by a method comprising:
(a) obtaining seed cells comprising natural killer cells from umbilical cord blood;
(b) depleting the seed cells of CD3+ cells;
(c) expanding the natural killer cells by culturing the depleted seed cells with a first plurality of Hut78 cells engineered to express a membrane bound IL-21, a mutated TNFα, and a 4-1BBL gene to produce expanded natural killer cells,
thereby producing the population of natural killer cells.

23. The method of any one of claims 10-22, wherein the population of NK cells is produced by a method comprising:
(a) obtaining seed cells comprising natural killer cells from umbilical cord blood;
(b) depleting the seed cells of CD3+ cells;
(c) expanding the natural killer cells by culturing the depleted seed cells with a first plurality of Hut78 cells engineered to express a membrane bound IL-21, a mutated TNFα, and a 4-1BBL gene to produce a master cell bank population of expanded natural killer cells; and
(d) expanding the master cell bank population of expanded natural killer cells by culturing with a second plurality of Hut78 cells engineered to express a membrane bound IL-21, a mutated TNFα, and a 4-1BBL gene to produce expanded natural killer cells;
thereby producing the population of natural killer cells.

24. The method of claim 22 or claim 23, wherein the population of NK cells is produced by a method further comprising, after step (c),
(i) freezing the master cell bank population of expanded natural killer cells in a plurality of containers; and
(ii) thawing a container comprising an aliquot of the master cell bank population of expanded natural killer cells,
wherein expanding the master cell bank population of expanded natural killer cells in step (d) comprises expanding the aliquot of the master cell bank population of expanded natural killer cells.

25. The method of any one of claims 22 to 24, wherein the umbilical cord blood is from a donor with the KIR-B haplotype and homozygous for the CD16 158V polymorphism.

26. The method of any one of claims 22-25, wherein the population of NK cells is produced by a method comprising expanding the natural killer cells from umbilical cord blood at least 10,000 fold, e.g., 15,000 fold, 20,000 fold, 25,000 fold, 30,000 fold, 35,000 fold, 40,000 fold, 45,000 fold, 50,000 fold, 55,000 fold, 60,000 fold, 65,000 fold, or 70,000 fold.

27. The method of any one of claims 22-26, wherein the population of natural killer cells is not enriched or sorted after expansion.

28. The method of any one of claims 22-27, wherein the percentage of NK cells expressing CD16 in the population of natural killer cells is the same or higher than the percentage of natural killer cells in the seed cells from umbilical cord blood.

29. The method of any one of claims 22-28, wherein the percentage of NK cells expressing NKG2D in the population of natural killer cells is the same or higher than the percentage of natural killer cells in the seed cells from umbilical cord blood.

30. The method of any one of claims 22-29, wherein the percentage of NK cells expressing NKp30 in the population of natural killer cells is the same or higher than the percentage of natural killer cells in the seed cells from umbilical cord blood.

31. The method of any one of claims 22-30, wherein the percentage of NK cells expressing NKp44 in the population of natural killer cells is the same or higher than the percentage of natural killer cells in the seed cells from umbilical cord blood.

32. The method of any one of claims 22-31, wherein the percentage of NK cells expressing NKp46 in the population of natural killer cells is the same or higher than the percentage of natural killer cells in the seed cells from umbilical cord blood.

33. The method of any one of claims 22-32, wherein the percentage of NK cells expressing DNAM-1 in the population of natural killer cells is the same or higher than the percentage of natural killer cells in the seed cells from umbilical cord blood.

34. The method of any of the forgoing claims, wherein the natural killer cell does not comprise a CD16 transgene.

35. The method of any of the forgoing claims, wherein the natural killer cell does not express an exogenous CD16 protein.

36. The method of any of the forgoing claims, wherein the natural killer cell is not genetically engineered.

37. The method of any of the forgoing claims, wherein the natural killer cell is derived from the same umbilical cord blood donor.

38. The method of any one of claims 1-37, wherein the first pharmaceutical composition further comprises:
(a) human albumin;
(b) dextran;
(c) glucose;
(d) DMSO; and
(e) a buffer.

39. The method of claim 38, wherein the first pharmaceutical composition comprises from 30 to 50 mg/mL human albumin.

40. The method of claim 38, wherein the first pharmaceutical composition comprises 50 mg/mL human albumin.

41. The method of any one of claims 38-40, wherein the first pharmaceutical composition comprises 20 to 30 mg/mL dextran.

42. The method of any one of claims 38-41, wherein the first pharmaceutical composition comprises 25 mg/mL dextran.

43. The method of any one of claims 38-42, wherein the dextran is Dextran 40.

44. The method of any one of claims 38-43, wherein the first pharmaceutical composition comprises from 12 to 15 mg/mL glucose.

45. The method of any one of claims 38-44, wherein the first pharmaceutical composition comprises 12.5 mg/mL glucose.

46. The method of any one of claims 38-45, wherein the first pharmaceutical composition comprises less than 27.5 g/L glucose.

47. The method of any one of claims 38-46, wherein the first pharmaceutical composition comprises from 50 to 60 ml/mL DMSO.

48. The method of any one of claims 38-47, wherein the first pharmaceutical composition comprises 55 mg/mL DMSO.

49. The method of any one of claims 38-48, wherein the first pharmaceutical composition comprises 40 to 60% v/v buffer.

50. The method of any one of claims 38-49, wherein the buffer is phosphate buffered saline.

51. The method of claim 1, wherein the first pharmaceutical composition further comprises:
(a) about 40 mg/mL human albumin;
(b) about 25 mg/mL Dextran 40;
(c) about 12.5 mg/mL glucose;
(d) about 55 mg/mL DMSO; and
(e) about 0.5 mL/mL phosphate buffered saline.

52. The method of any one of claims 38-51, wherein the first pharmaceutical composition further comprises 0.5 mL/mL water.

53. The method of any one of claims 1-37, wherein the first pharmaceutical composition further comprises a pharmaceutically acceptable excipient.

54. The method of any one of claims 1-53, wherein the first binding domain that specifically binds to CD16 comprises a light chain variable (V_{L}) region comprising SEQ ID NO: 20 and a heavy chain variable (V_{H}) region comprising SEQ ID NO: 19.

55. The method of any one of claims 1-54, wherein the first binding domain that specifically binds to CD16 comprises a V_{L} region comprising an amino acid sequence having or having at least 85%, 86%, 87%, 88%, 89%, 90%, 91%, 92%, 93%, 94%, 95%, 96%, 97%, 98%, or 99% identity to SEQ ID NO: 20 and a V_{H} region comprising an amino acid sequence having or having at least 85%, 86%, 87%, 88%, 89%, 90%, 91%, 92%, 93%, 94%, 95%, 96%, 97%, 98%, or 99% identity to SEQ ID NO: 19.

56. The method of any one of claims 1-55, wherein the second binding domain that specifically binds to CD30 comprises a light chain variable (V_{L}) region comprising SEQ ID NO: 22 and a heavy chain variable (V_{H}) region comprising SEQ ID NO: 21.

57. The method of any one of claims 1-56, wherein the second binding domain that specifically binds to CD30 comprises a V_{L} region comprising an amino acid sequence having or having at least 85%, 86%, 87%, 88%, 89%, 90%, 91%, 92%, 93%, 94%, 95%, 96%, 97%, 98%, or 99% identity to SEQ ID NO: 22 and a V_{H} region comprising an amino acid sequence having or having at least 85%, 86%, 87%, 88%, 89%, 90%, 91%, 92%, 93%, 94%, 95%, 96%, 97%, 98%, or 99% identity to SEQ ID NO: 21.

58. The method of any one of claims 1-57, wherein:
the bispecific antibody or antigen binding fragment thereof is a bispecific antigen binding fragment; and
wherein the variable domains of the bispecific antigen binding fragment are linked by peptide linkers L1, L2, and L3 from the N-terminus to the C-terminus in the order: VH_CD30-L1-VL_CD16A-L2-VH_CD16A-L3-VL_CD30.

59. The method of any one of claims 1-57, wherein:
the bispecific antibody or antigen binding fragment thereof is a bispecific antigen binding fragment; and
wherein the variable domains of the bispecific antigen binding fragment are linked by peptide linkers L1, L2, and L3 from the N-terminus to the C-terminus in the order: VH_CD16A-L1-VL_CD30-L2-VH_CD30-L3-VL_CD16A.

60. The method of claim 58 or 59, wherein each of peptide linkers L1, L2, and L3 consist of no more than 12 amino acid residues.

61. The method of any one of claims 57 to 60, wherein linker L2 of the antibody construct consists of between 3 and 9 amino acid residues, inclusive.

62. The method of any one of claims 1-61, wherein the bispecific antibody or antigen binding fragment thereof is a comprises an amino acid sequence having or having at least 85%, 86%, 87%, 88%, 89%, 90%, 91%, 92%, 93%, 94%, 95%, 96%, 97%, 98%, or 99% identity to SEQ ID NO: 18.

63. The method of any one of claims 1-62, wherein the bispecific antibody or antigen binding fragment thereof is a bispecific antigen binding fragment comprising an amino acid sequence set forth in SEQ ID NO: 18.

64. The method of any one of claims 1-63, wherein a dose of the bispecific antibody or antigen binding fragment thereof is administered to the patient at 0.01, 0.04, 0.15, 0.5, 1.5, 3.0, 4.5 or 7.0 mg/kg.

65. The method of any one of claims 1-63, wherein a dose of the bispecific antibody or antigen binding fragment thereof comprises 200 mg of the bispecific antibody or antigen binding fragment thereof.

66. The method of any one of claims 1-63, wherein the cancer is selected from the group consisting of Hodgkin lymphoma, non-Hodgkin lymphoma, peripheral T-cell lymphoma, cutaneous T cell lymphoma, anaplastic large-cell lymphoma, CD30+B-cell lymphoma, multiple myeloma, and leukemia.

67. The method of claim 66, wherein the cancer is Hodgkin lymphoma.

68. The method of claim 66, wherein the cancer is peripheral T-cell lymphoma.

69. The method of any one of claims 1-68, wherein the patient has relapsed after treatment with or is refractory to an anti-CD30 antibody.

70. The method of claim 69, wherein the anti-CD30 antibody is brentuximab vedotin.

71. The method of any one of claims 1-70, wherein the patient has experienced disease progression after treatment with autologous stem cell transplant or chimeric antigen receptor T-cell therapy (CAR-T).

72. The method of any one of claims 1-71, wherein the patient is administered 1×10⁸ to 1×10¹⁰ NK cells per dose of NK cells.

73. The method of claim 72, wherein the patient is administered 1×10⁹ to 8×10⁹ NK cells per dose of NK cells.

74. The method of claim 72, wherein the patient is administered 4×10⁸, 1×10⁹, 4×10⁹, 8×10⁹ NK, or 1.6×10¹⁰ cells per dose of NK cells.

75. The method of any of the forgoing claims, wherein the patient is subjected to lymphodepleting chemotherapy prior to treatment.

76. The method of claim 75, wherein the lymphodepleting chemotherapy is non-myeloablative chemotherapy.

77. The method of claim 75 or claim 76, wherein the lymphodepleting chemotherapy comprises treatment with at least one of cyclophosphamide and fludarabine.

78. The method of claim 77, wherein the lymphodepleting chemotherapy comprises treatment with cyclophosphamide and fludarabine.

79. The method of any one of claims 77-78, wherein the cyclophosphamide is administered between 100 and 500 mg/m²/day.

80. The method of claim 79, wherein the cyclophosphamide is administered at 250 mg/m²/day.

81. The method of claim 79, wherein the cyclophosphamide is administered at 500 mg/m²/day.

82. The method of any one of claims 77-81, wherein the fludarabine is administered between 10 and 50 mg/m²/day.

83. The method of claim 82, wherein the fludarabine is administered at 30 mg/m²/day.

84. The method of any of the forgoing claims further comprising administering IL-2 to the patient.

85. The method of claim 84, wherein the patient is administered 1×10⁶ IU/m² of IL-2 per dose.

86. The method of claim 84, wherein the patient is administered 1 million or 6 million IU of IL-2 per dose.

87. The method of any one of claims 84-86, wherein administration of IL-2 occurs within 1-4 hours of administration of the NK cells.

88. The method of any of the forgoing claims, wherein the administration of a dose of the first pharmaceutical composition comprising the NK cell and a dose of the second pharmaceutical composition comprising the bispecific antibody or antigen binding fragment thereof occurs weekly.

89. The method of any of the forgoing claims, wherein the NK cells and the first pharmaceutical composition comprising the NK cell and the second pharmaceutical composition comprising the bispecific antibody or antigen binding fragment thereof are administered weekly for 4 to 8 weeks.

90. The method of any of the forgoing claims, wherein the administration of the first pharmaceutical composition comprising the NK cell occurs weekly for three weeks and the second pharmaceutical composition comprising the bispecific antibody or antigen binding fragment thereof occurs weekly for six weeks.

91. The method of any of the forgoing claims, wherein the administration of the first pharmaceutical composition comprising the NK cell occurs every other week for six weeks and the second pharmaceutical composition comprising the bispecific antibody or antigen binding fragment thereof occurs weekly for six weeks.

92. A method for treating a patient suffering from a CD30+ cancer, the method comprising:
administering to the patient a first cycle of treatment comprising the method of any one of claims 1-91; and
administering to the patient a second cycle of treatment comprising the method of any one of claims 1-91,
wherein the first cycle of treatment and the second cycle of treatment are the same or different.

93. The method of claim 92, further comprising administering to the patient a third cycle of treatment comprising the method of any one of claims 1-91.

94. The method of claim 92 or claim 93, wherein the method comprises a treatment break of at least two weeks between cycles.

95. The method of any one of claim 92, wherein the treatment continues until the CD30+ cancer progresses, or until the doses are discontinued due to the patient's intolerance of the NK cell, the bispecific antibody or antigen binding fragment thereof, or both, or until the patient experiences toxicity the NK cells, the bispecific antibody or antigen binding fragment thereof, or both.

96. The method of any of the forgoing claims, wherein the NK cells are not genetically modified.

97. The method of any of the forgoing claims, wherein at least 70% of the NK cells are CD56+ and CD16+.

98. The method of any of the forgoing claims, wherein at least 85% of the NK cells are CD56+ and CD3−.

99. The method of any of the forgoing claims, wherein 1% or less of the NK cells are CD3+, 1% or less of the NK cells are CD19+ and 1% or less of the NK cells are CD14+.

100. A pharmaceutical composition comprising:
(a) a natural killer cell (NK cell) comprising a KIR-B haplotype and expression of a CD16 molecule; and
(b) a bispecific antibody or antigen binding fragment thereof comprising a first binding domain that specifically binds to CD16 (FcγRIII) and a second binding domain that specifically binds to CD30,
wherein the first binding domain that specifically binds to CD16 comprises:
a light chain variable domain (VL_CD16A) comprising a light chain complementarity determining region 1 (CDRL1) comprising SEQ ID NO: 9, a light chain complementarity determining region 2 (CDRL2) comprising SEQ ID NO: 10; a light chain complementarity determining region 3 (CDRL3) comprising SEQ ID NO: 11; and
a heavy chain variable domain (VH_CD16A) comprising a heavy chain complementarity determining region 1 (CDRH1 comprising SEQ ID NO: 6; a heavy chain complementarity determining region 2 (CDRH2) comprising SEQ ID NO: 7; and a heavy chain complementarity determining region 3 (CDRH3) comprising SEQ ID NO: 8; and
wherein the second binding domain that specifically binds to CD30 comprises:
a light chain variable domain (VL_CD30) comprising a light chain complementarity determining region 1 (CDRL1) comprising SEQ ID NO: 15, a light chain complementarity determining region 2 (CDRL2) comprising SEQ ID NO: 16; a light chain complementarity determining region 3 (CDRL3) comprising SEQ ID NO: 17; and
a heavy chain variable domain (VH_CD30) comprising a heavy chain complementarity determining region 1 (CDRH1 comprising SEQ ID NO: 12; a heavy chain complementarity determining region 2 (CDRH2) comprising SEQ ID NO: 13; and a heavy chain complementarity determining region 3 (CDRH3) comprising SEQ ID NO: 14.

101. The pharmaceutical composition of claim 100, wherein the CD16 molecule is a CD16A molecule.

102. The pharmaceutical composition of claim 100 or claim 101, wherein the CD16 molecule comprises a V/V polymorphism at F158.

103. The pharmaceutical composition of any one of claims 100-102, wherein the bispecific antibody that specifically binds to CD16 specifically binds to CD16A.

104. The pharmaceutical composition of any one of claims 100-103, wherein the first binding domain that specifically binds to CD16 comprises a light chain variable (V_{L}) region comprising SEQ ID NO: 20 and a heavy chain variable (V_{H}) region comprising SEQ ID NO: 19.

105. The pharmaceutical composition of any one of claims 100-104, wherein the first binding domain that specifically binds to CD16 comprises a V_{L} region comprising an amino acid sequence having or having at least 85%, 86%, 87%, 88%, 89%, 90%, 91%, 92%, 93%, 94%, 95%, 96%, 97%, 98%, or 99% identity to SEQ ID NO: 20 and a V_{H} region comprising an amino acid sequence having or having at least 85%, 86%, 87%, 88%, 89%, 90%, 91%, 92%, 93%, 94%, 95%, 96%, 97%, 98%, or 99% identity to SEQ ID NO: 19.

106. The pharmaceutical composition of any one of claims 100-105, wherein the second binding domain that specifically binds to CD30 comprises a light chain variable (V_{L}) region comprising SEQ ID NO: 22 and a heavy chain variable (V_{H}) region comprising SEQ ID NO: 21.

107. The pharmaceutical composition of any one of claims 100-106, wherein the second binding domain that specifically binds to CD30 comprises a V_{L} region comprising an amino acid sequence having or having at least 85%, 86%, 87%, 88%, 89%, 90%, 91%, 92%, 93%, 94%, 95%, 96%, 97%, 98%, or 99% identity to SEQ ID NO: 22 and a V_{H} region comprising an amino acid sequence having or having at least 85%, 86%, 87%, 88%, 89%, 90%, 91%, 92%, 93%, 94%, 95%, 96%, 97%, 98%, or 99% identity to SEQ ID NO: 21.

108. The pharmaceutical composition of any one of claims 100-107, wherein:
the bispecific antibody or antigen binding fragment thereof is a bispecific antigen binding fragment; and
wherein the variable domains of the bispecific antigen binding fragment are linked by peptide linkers L1, L2, and L3 from the N-terminus to the C-terminus in the order: VH_CD30-L1-VL_CD16A-L2-VH_CD16A-L3-VL_CD30.

109. The pharmaceutical composition of any one of claims 100-107, wherein:
the bispecific antibody or antigen binding fragment thereof is a bispecific antigen binding fragment; and
wherein the variable domains of the bispecific antigen binding fragment are linked by peptide linkers L1, L2, and L3 from the N-terminus to the C-terminus in the order: VH_CD16A-L1-VL_CD30-L2-VH_CD30-L3-VL_CD16A.

110. The pharmaceutical composition of claim 108 or claim 109, wherein each of peptide linkers L1, L2, and L3 consist of no more than 12 amino acid residues.

111. The pharmaceutical composition of any one of claims 108 to 110, wherein linker L2 of the antibody construct consists of between 3 and 9 amino acid residues, inclusive.

112. The pharmaceutical composition of any one of claims 100-111, wherein the bispecific antibody or antigen binding fragment thereof is a comprises an amino acid sequence having or having at least 85%, 86%, 87%, 88%, 89%, 90%, 91%, 92%, 93%, 94%, 95%, 96%, 97%, 98%, or 99% identity to SEQ ID NO: 18.

113. The pharmaceutical composition of any one of claims 100-112, wherein the bispecific antibody or antigen binding fragment thereof is a bispecific antigen binding fragment comprising an amino acid sequence set forth in SEQ ID NO: 18.

114. The pharmaceutical composition of any one of claims 100-113, wherein the NK cell is a cord blood-derived NK cell.

115. The pharmaceutical composition of claim 114, wherein the cord blood-derived NK cell has been produced by a method comprising:
(a) providing a sample of cord blood cells comprising natural killer cells;
(b) depleting the cells of CD3 (+) cells;
(b) expanding the natural killer cells by culturing the seed cells with a first plurality of cells from an inactivated CD4 (+) T cell line in a medium comprising:
a T-cell stimulating antibody selected from the group consisting of OKT3, UCHT1, HTa, or a combination thereof; and
IL-2,
to produce the cord blood-derived natural killer cells.

116. The pharmaceutical composition of claim 115, wherein the inactivated CD4 (+) T cell line expresses at least one gene selected from the group consisting of a 4-1BBL gene, a membrane-bound IL-21 (mbIL-21) gene, an OX40L gene, and a mouse TNF-α gene.

117. The pharmaceutical composition of claim 116, wherein the inactivated CD4 (+) T cell line expresses a 4-1BBL gene, a membrane-bound IL-21 (mbIL-21) gene, and a mouse TNF-α gene.

118. The pharmaceutical composition of any one of claims 100-117, wherein the first binding domain that specifically binds to CD16 of the bispecific antibody or antigen binding fragment thereof is bound to the CD16 molecule of the NK cell.

119. The pharmaceutical composition of any one of claims 100-118, further comprising
(a) human albumin;
(b) dextran;
(c) glucose;
(d) DMSO; and
(e) a buffer.

120. The pharmaceutical composition of claim 119 comprising from 30 to 50 mg/mL human albumin.

121. The pharmaceutical composition of claim 120 comprising 50 mg/mL human albumin.

122. The pharmaceutical composition of any one of claims 119-121 comprising 20 to 30 mg/mL dextran.

123. The pharmaceutical composition of any one of claims 119-122 comprising 25 mg/mL dextran.

124. The pharmaceutical composition of any one of claims 119-123, wherein the dextran is Dextran 40.

125. The pharmaceutical composition of any one of claims 119-124 comprising from 12 to 15 mg/mL glucose.

126. The pharmaceutical composition of any one of claims 119-125 comprising 12.5 mg/mL glucose.

127. The pharmaceutical composition of any one of claims 119-126 comprising less than 27.5 g/L glucose.

128. The pharmaceutical composition of any one of claims 119-127 comprising from 50 to 60 ml/mL DMSO.

129. The pharmaceutical composition of any one of claims 119-128 comprising 55 mg/mL DMSO.

130. The pharmaceutical composition of any one of claims 119-129 comprising 40 to 60% v/v buffer.

131. The pharmaceutical composition of any one of claims 119-130, wherein the buffer is phosphate buffered saline.

132. The pharmaceutical composition of claim 119 comprising:
(a) about 40 mg/mL human albumin;
(b) about 25 mg/mL Dextran 40;
(c) about 12.5 mg/mL glucose;
(d) about 55 mg/mL DMSO; and
(e) about 0.5 mL/mL phosphate buffered saline.

133. The pharmaceutical composition of any one of claims 119-132, further comprising 0.5 mL/mL water.

134. The pharmaceutical composition of any one of claims 100-118, further comprising a pharmaceutically acceptable excipient.

135. A frozen vial comprising the pharmaceutical composition of any one of claims 100-134.

136. A method for treating a patient suffering from a CD30+ cancer, the method comprising administering the pharmaceutical composition of any one of claims 100-134.


SEQUENCE LISTING (28 sequences)

SEQ ID NO: 1 - protein, 254 aa, unidentified
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 2 - protein, 223 aa, unidentified
MALPVTALLLPLALLLHAARPQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSAKPTTTPAPRPPTPAPTIASQPLSLRPEACRPAAGGAVHTRGLDFACDIYIWAPLAGTCGVLLLSLVITLY

SEQ ID NO: 3 - protein, 233 aa, unidentified
MSTESMIRDVELAEEALPKKTGGPQGSRRCLFLSLFSFLIVAGATTLFCLLHFGVIGPQREEFPRDLSLISPLAQPVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 4 - protein, 183 aa, unidentified
MERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 5 - protein, 609 aa, unidentified [sequence omitted]

SEQ ID NO: 6 - protein, 5 aa, unidentified
SYYMH

SEQ ID NO: 7 - protein, 17 aa, unidentified
IINPSGGSTSYAQKFQG

SEQ ID NO: 8 - protein, 11 aa, unidentified
GSAYYYDFADY

SEQ ID NO: 9 - protein, 11 aa, unidentified
GGHNIGSKNVH

SEQ ID NO: 10 - protein, 7 aa, unidentified
QDNKRPS

SEQ ID NO: 11 - protein, 9 aa, unidentified
QVWDNYSVL

SEQ ID NO: 12 - protein, 5 aa, unidentified
TYTIH

SEQ ID NO: 13 - protein, 17 aa, unidentified
YINPSSGYSDYNQNFKG

SEQ ID NO: 14 - protein, 14 aa, unidentified
RADYGNYEYTWFAY

SEQ ID NO: 15 - protein, 11 aa, unidentified
KASQNVGTNVA

SEQ ID NO: 16 - protein, 7 aa, unidentified
SASYRYS

SEQ ID NO: 17 - protein, 9 aa, unidentified
QQYHTYPLT

SEQ ID NO: 18 - protein, 483 aa, unidentified
QVQLVQSGAEVKKPGESLKVSCKASGYTFTSYYMHWVRQAPGQGLEWMGIINPSGGSTSYAQKFQGRVTMTRDTSTSTVYMELSSLRSEDTAVYYCARGSAYYYDFADYWGQGTLVTVSSGGSGGSGGSDIVMTQSPKFMSTSVGDRVTVTCKASQNVGTNVAWFQQKPGQSPKVLIYSASYRYSGVPDRFTGSGSGTDFTLTISNVQSEDLAEYFCQQYHTYPLTFGGGTKLEINGGSGGSGGSQVQLQQSGAELARPGASVKMSCKASGYTFTTYTIHWVRQRPGHDLEWIGYINPSSGYSDYNQNFKGKTTLTADKSSNTAYMQLNSLTSEDSAVYYCARRADYGNYEYTWFAYWGQGTTVTVSSGGSGGSGGSSYVLTQPSSVSVAPGQTATISCGGHNIGSKNVHWYQQRPGQSPVLVIYQDNKRPSGIPERFSGSNSGNTATLTISGTQAMDEADYYCQVWDNYSVLFGGGTKLTVL

SEQ ID NO: 19 - protein, 120 aa, unidentified
QVQLVQSGAEVKKPGESLKVSCKASGYTFTSYYMHWVRQAPGQGLEWMGIINPSGGSTSYAQKFQGRVTMTRDTSTSTVYMELSSLRSEDTAVYYCARGSAYYYDFADYWGQGTLVTVSS

SEQ ID NO: 20 - protein, 106 aa, unidentified
SYVLTQPSSVSVAPGQTATISCGGHNIGSKNVHWYQQRPGQSPVLVIYQDNKRPSGIPERFSGSNSGNTATLTISGTQAMDEADYYCQVWDNYSVLFGGGTKLTVL

SEQ ID NO: 21 - protein, 123 aa, unidentified
QVQLQQSGAELARPGASVKMSCKASGYTFTTYTIHWVRQRPGHDLEWIGYINPSSGYSDYNQNFKGKTTLTADKSSNTAYMQLNSLTSEDSAVYYCARRADYGNYEYTWFAYWGQGTTVTVSS

SEQ ID NO: 22 - protein, 107 aa, unidentified
DIVMTQSPKFMSTSVGDRVTVTCKASQNVGTNVAWFQQKPGQSPKVLIYSASYRYSGVPDRFTGSGSGTDFTLTISNVQSEDLAEYFCQQYHTYPLTFGGGTKLEIN

SEQ ID NO: 23 - protein, 8 aa, unidentified
SFFPPGYQ

SEQ ID NO: 24 - protein, 192 aa, unidentified
GMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQ

SEQ ID NO: 25 - protein, 6 aa, unidentified
HHHHHH

SEQ ID NO: 26 - protein, 9 aa, unidentified
GGSGGSGGS

SEQ ID NO: 27 - protein, 9 aa, unidentified
GGSGGSGGS

SEQ ID NO: 28 - protein, 9 aa, unidentified
GGSGGSGGS